(19) United States
(12) Patent Application Publication
(10) Pub. No.: US 2023/0255974 A1
(43) Pub. Date: Aug. 17, 2023

(54) USE OF THYROMIMETICS FOR THE TREATMENT OF CANCER

(71) Applicant: THE UNIVERSITY OF VERMONT, Burlington, VT (US)

(72) Inventor: Frances E. CARR, Shelburne, VT (US)

(21) Appl. No.: 17/907,916

(22) PCT Filed: Mar. 1, 2021
(86) PCT No.: PCT/US2021/020282

§ 371 Date: Aug. 29, 2022

Related U.S. Application Data
(60) Provisional application No. 62/983,628, filed on Feb. 29, 2020.

(51) Int. Cl. A61K 31/5377; A61P 35/00; A61K 45/06

(52) U.S. Cl. CPC A61K 31/5377; A61P 35/00; A61K 45/06

(57) ABSTRACT

The present application is directed to a combination therapy comprising a thyroid hormone receptor beta-1 (TKβ) agonist, and a primary cancer therapeutic. Methods of treating cancer and inducing differentiation in a population of cancer cells are also disclosed.

This application claims the priority benefit of U.S. Provisional Patent Application Ser. No. 62/983,628, filed Feb. 29, 2020, which is hereby incorporated by reference in its entirety.

### FIELD

The present invention is directed to methods and compositions comprising thyromimetics for use in the treatment of cancer.

### BACKGROUND

Thyroid cancer is the most common endocrine cancer worldwide, and the global incidence has increased faster than any other solid tumor over the past few decades (Jemal et al. “Global cancer statistics,” CA Cancer J Clin 61:69-90 (2011); Pellegriti et al., “Worldwide increasing incidence of thyroid cancer: update on epidemiology and risk factors,” J Cancer Epidemiol 2013:965212 (2013); Zhu et al., “A birth cohort analysis of the incidence of papillary thyroid cancer in the United States, 1973-2004,” Thyroid 19:1061-1066 (2009); Lim et al., “Trends in thyroid cancer incidence and mortality in the united states, 1974-2013,” JAMA 317:1338-1348 (2017)). While the prognoses for patients with differentiated tumors receiving standard therapies are generally very good, outcomes for patients with resistant or recurrent disease and poorly differentiated thyroid cancers (PDTC) are extremely poor. Due to a lack of effective therapies, patients with advanced or metastatic anaplastic thyroid cancer (ATC) have a higher mortality rate than all other endocrine cancers combined with a mean survival time of 6 months (Lim et al., “Trends in thyroid cancer incidence and mortality in the united states, 1974-2013,” JAMA 317:1338-1348 (2017) and Molinaro et al., “Anaplastic thyroid carcinoma: from clinicopathology to genetics and advanced therapies,” Nature Reviews Endocrinology 13:644 (2017)). The pathogenesis of thyroid cancer is characterized by dysregulation of intracellular signaling pathways, abnormalities in expression of tumor suppressors, cell cycle regulators, and apoptotic signaling. ATC tumors are refractory to radioiodine therapy due to loss of sodium-iodide symporter, and traditional chemotherapy is of limited benefit. Treatment of PDTC and ATC with standard of care intracellular signaling inhibitors rarely provide a durable response due to development of resistance. Thus, there is an urgent need for more targeted precision-based therapeutic options.

Breast cancer (BCa) is the most commonly diagnosed cancer in women worldwide (Ferlay et al., “Cancer incidence and mortality worldwide: sources, methods and major patterns in GLOBOCAN,” Int J Cancer 2015; 136:E359-386 (2012)). Endocrine therapies that target the estrogen receptor alpha (ERα) are the cornerstone of BCa treatment for the majority of patients with ER+ disease. Nonetheless, these therapies often fail, in both the curative as well as metastatic setting, as a consequence of the development of various resistance pathways that are still being elucidated. There are limited treatment options for the less common triple negative BCa (TNBC) subtypes. Steroid hormone receptor and HER2 receptor signatures define therapeutic strategies in BCa (Finlay-Schultz and Sartorius, “Steroid hormones, steroid receptors, and breast cancer stem cells,” J Mammary Gland Biol Neoplasia 20:39-50 (2015) and Folkerd and Dowsett, “Sex hormones and breast cancer risk and prognosis,” Breast 22 Suppl 2:S38-43 (2013)), but there is a need for more targeted and precision-based treatment options.

The present invention is directed at overcoming these deficiencies in the field by providing a combination therapy for the treatment of early stage, aggressive, and treatment-resistant forms of cancer.

### SUMMARY

A first aspect of the present invention is directed to a combination therapy comprising a thyroid hormone receptor beta-1 (TRβ) agonist, and a primary cancer therapeutic.

Another aspect of the present invention is directed to a method for treating cancer in a subject. This method involves administering to a subject having a cancer, wherein the cancer is characterized by cells having a decreased level of thyroid hormone receptor beta-1 (TRβ) expression or activity relative to corresponding non-cancer cells of similar origin, a TRβ agonist in an amount effective to treat the cancer.

Another aspect of the present invention is directed to a method of inducing differentiation in a population of cancer cells. This method involves administering to a population of cancer cells having a decreased level of thyroid hormone receptor beta-1 (TRβ) expression or activity relative to a corresponding population of non-cancer cells of similar origin, and a TRβ agonist in an amount effective to induce differentiation of the cancer cells of the population.

As disclosed herein activation of TRβ1 inhibits tumorigenic signaling, reduces the aggressive phenotype of cancer cells, enhances redifferentiation of cancer cells, and increases sensitivity of cancer cells to known and novel therapies. TRβ1 as a tumor suppressor is tumor agnostic and functions in thyroid, breast and other solid tumors and is a key biomarker and therapeutic target in solid tumors.

### BRIEF DESCRIPTION OF THE DRAWINGS

FIGS. 1A-1D show that TRβ Represses Growth of SW1736. The combination of TRβ expression and T₃ treatment repressed the growth of the ATC cells (n=8) (FIG. 1A). Variability is represented by the standard error of the mean, significance was determined by calculating the 95^{th} confident interval of AUC measurements. TRβ and T₃ acted to increase doubling time of the cells (n=8, * p<0.01) as shown in FIG. 1B. FIG. 1C shows that SW-TRβ treated with T₃ exhibited significantly reduced scratch closure at the 95^{th} confidence interval by AUC (n=9), and FIG. 1D are representative images.

FIGS. 2A-2C show TRβ-T₃ alters the transcriptome of ATC cells. Thresholds for differentially regulated genes (DEGs) were set at p<0.05 and an absolute log 2foldchange of at least 1, upregulated transcripts in red and repressed transcripts in blue (FIG. 2A). Genes were clustered according to patterns of expression. Clusters 1 and 5 are genes that are T₃ regulated independent of TRβ overexpression. Clusters 2-4 require TRβ for T₃ to exert a regulatory effect. Ingenuity Pathway Analysis (IPA) software was utilized to determine pathways altered within each cluster. Notable cancer-related pathways are highlighted (FIG. 2B). IPA software was used to ascertain upstream regulators within each cluster (FIG. 2C). Exogenous chemicals were excluded, however endogenous chemicals were retained.

FIGS. 3A-3J show TRβ represses PI3K signaling. FIGS. 3A-3I are graphs showing expression of cluster 1 differentially expressed genes in the ILK signaling IPA pathway. All genes were repressed by T₃ and the level of most of these genes was repressed in the SW-TRβ-T₃ condition compared to SW-EV-T₃. (n=3, * p<0.05, n.s. p>0.05). FIG. 3J shows AKT phosphorylation was repressed upon overexpression of TRβ in SW1736 cells. (representative blot of 3 experiments)

FIGS. 4A-4B shows the results of RNA-sequencing of the TRβ-Regulated Transcriptome in ATC cells. Chromatin Enrichment Analysis (ChEA) of differential expression clusters was performed to evaluate which transcription factor networks were overrepresented in each cluster (FIG. 4A). Multiple transcription factors elucidated by ChEA were also differentially expressed (FIG. 4B).

FIGS. 5A-5B shows that TRβ regulates expression of lncRNAs. A pairwise comparison between SW-EV-T₃ and SW-TRβ-T₃ reveals differential expression of both mRNAs and lncRNAs (log₂(foldchange)>2 and p<0.05) (FIG. 5A). A total of 350 mRNAs and 28 lncRNAs were differentially expressed. FIG. 5B shows expression levels of the differentially expressed lncRNAs in transcripts per million reads.

FIGS. 6A-6E show that stem cell characteristics were reduced by TRβ. Treatment of SW1736 cells with T₃ was sufficient to decrease anchorage-independent growth and colony growth; the most pronounced effect was observed in the SW-TRβ cells (n=4, * p<0.05) (FIGS. 6A and 6B). T₃ significantly repressed tumorsphere formation in SW-TRβ, but not SW-EV (n=4, * p<0.05) (FIG. 6C). Thyroid cancer-specific stem cell marker mRNA transcript levels were significantly repressed by TRβ and T₃ by RNA-seq (n=3, * p<0.05) (FIG. 6D). Reintroduction of TRβ and treatment with T₃ resulted in a significantly increased thyroid differentiation score (TDS) (n=3, * p<0.05) (FIG. 6E).

FIG. 7 shows TRβ induces a more differentiated phenotype. RNA-sequencing data revealed statistically significant increase of the epithelial marker CDH1 (left graph) and repression of the mesenchymal marker VIM (middle graph). Although SNAI1 was expressed, no significant changes in expression were observed (right graph). n=3, * p<0.05, ***** p<0.0001

FIG. 8 shows reintroduction of TRβ alters thyroid differentiation markers. Thyroid differentiation markers were significantly induced in SW-TRβ cells treated with T₃ (n=3, * p<0.05).

FIGS. 9A-9C show TRβ reintroduction increases apoptotic signaling. In a comparison of DEGs between SW-EV and SW-TRβ, both T₃ treated, IPA predicted changes in pathways important to apoptotic signaling (FIG. 9A), and GSEA predicted activation of apoptosis (FIG. 9B). The representative immunoblot of FIG. 9C demonstrates that 5 days of T₃ treatment induces apoptosis in SW-TRβ cells assessed by cleavage of PARP1 and caspase 3 (n=6).

FIGS. 10A-10D show that the Interferon-JAK1-STAT1 pathway is activated by TRβ. Interferon pathway effector proteins JAK1 and STAT1 are expressed at higher levels in SW-TRβ cells following T₃ treatment (*p<0.05, n=6) (FIG. 10A). STAT1 and IRF1 genes were induced upon overexpression of TRβ and T₃ treatment. (n=6, * p<0.05, ** p<0.01, *** P<0.001, **** p<0.0001) (FIG. 10B). Baseline expression of key genes in the JAK1-STAT1 pathway and TR isoform levels (TRβ and TRα) in 3 additional anaplastic thyroid cancer cell lines are illustrated (FIG. 10C). STAT1 activator 2-(1,8-naphthyridin-2-yl)phenol (2-NP) recapitulates the T₃-TRβ induced reduction in cell growth confirming the T₃-TRβ effect on the JAK1-STAT1 pathway (FIG. 10D). The SW1736 cells were most responsive to this modulation which may reflect TP53 truncation and basal expression of effector proteins. Cells were treated with the indicated concentrations of 2-NP for 24 hr. Data are mean+/−SD, n=3.

FIG. 11 shows the reintroduction of TRβ alters expression of the interferon/JAK1/STAT1 pathway. Following 24 hours of treatment with T₃ or vehicle, protein levels in SW-EV and SW-TRβ were assessed by western blot. Representative western blot depicted.

FIG. 12 is a schematic of the major pathways altered by TRβ. TRβ stimulates activity of STAT1, a transducer of anti-proliferative signaling. Stemness of the cells is reduced, while thyroid differentiation markers are increased. Additionally, apoptosis was increased in the cells.

FIGS. 13A-13D show that GC-1 induced anaplastic thyroid cancer cell death and amplifies the effect of TRβ expression. SW1736 cells were transduced with a lentiviral vector (EV) or a lentiviral vector containing the TRβ cDNA (TRβ). Growth in absence of TRβ (FIG. 13A) and presence of TRβ (FIG. 13B) reveals that re-expression of TRβ in ATC cells (SW1736) induces cell death. Activation of endogenous TRβ with GC-1 (10⁻⁸M) induces ATC cell death (FIG. 13C); an effect that is amplified with higher levels of TRβ (FIG. 13D) achieved by lentiviral transduction.

FIGS. 14A-14B show activation of TRβ by ligand triiodothyronine (T₃) or selective agonist sobetirome (GC-1) induces apoptosis in anaplastic thyroid cancer cells (SW1736) through the JAK1-STAT1 signaling pathway. TRβ was over-expressed in SW1736 cells by lentiviral transduction (SW-TRβ) and compared with cells transduced with an empty vector control (SW-EV). Cells were treated with either T₃ or GC-1 for five days. Apoptosis (schematically represented in FIG. 14A) was seen in treated cells in which TRβ levels are detected. In both treatments, TRβ induces apoptosis in part through JAK1-STAT1 signaling as reflected by cleaved PARP and caspase illustrated in the above immunoblots (FIG. 14B).

FIGS. 15A-15B show PI3K-Akt signaling pathway in thyroid cancer. Increased PI3K-Akt pathway activity is a hallmark of thyroid, breast and other solid tumors and the signaling pathway is schematically represented in FIG. 15A. FIG. 15B shows schematically how PI3K inhibitors, buparlisib and LY294002 block PI3k activity.

FIGS. 16A-16B show expression of TRβ enhances the efficacy of PI3K inhibitor LY294002. SW1736 ATC cells were transduced with either empty vector (EV) or vector with TRβ (TRβ) and treated with LY294002 for 24 hr in the presence of ligand T₃ (10⁻⁸ M). PI3K activity is reflected by targets phosphorylated Akt (pAkt) and phosphorylated mTOR (p mTOR). The presence of TRβ decreases PI3K activity and further increases the effectiveness of LY294002 as reflected by decreased pAkt compared with total Akt and p mTOR compared with total mTOR detected by immunoblot (FIG. 16A). Quantitation of the immunoblot is shown (FIG. 16B). Data reflect 3 independent experiments.

FIGS. 17A-17B show TRβ enhances PI3K inhibitor LY294002 inhibition of ATC cell growth and cell migration. The presence of liganded TRβ (SW-TRβ) increases the efficacy of LY294002 in ATC cells compared without TRβ re-expressed (SW-EV) with an EC₅₀ of 1.13 compared with 5.21 μM, respectively (FIG. 17A). LY294002 induces a 50% reduction in growth of normal-like thyroid cells NthyOri in which TRβ is expressed. The presence of liganded TRβ enhances LY294002 inhibition of ATC cell migration (FIG. 17B). Data are mean+/−SD, n=6.

FIG. 18 shows TRβ expression increases the efficacy of the PI3K inhibitor buparlisib. SW1736 cells transduced with lentiviral vector (EV) or lentiviral vector containing TRβ (TRβ) were treated with buparlisib for 24 hr with the indicated concentrations. Buparlisib alone causes a concentration dependent decrease in cell viability (EV). The presence of TRβ (TRβ) increases the sensitivity of the cells to buparlisib resulting in a significantly reduced EC50.

FIG. 19 show activation of TRβ with selective agonist GC-1 enhances buparlisib inhibition of ATC cell growth. SW-EV or SW-TRβ cells were treated with GC-1 (10⁻⁸M) for 24 hr in the absence or presence of increasing concentrations of buparlisib. As noted, TRβ (TRβ) decreases cell growth in the absence of buparlisib. The addition of GC-1 with TRβ further reduces cell growth. GC-1 alone (EV+GC-1) inhibits cell growth through the low levels of endogenous TRβ. TRβ and GC-1 increase the sensitivity to buparlisib at 0.1 and 1 μM. Buparlisib at 10 μM is toxic. Data are mean+/−SD; n=6.

FIGS. 20A-20D show GC-1 blocks tumorigenic phenotypes in ATC cells transduced with TRβ. FIG. 20A shows growth curves that demonstrate that SW-TRβ growth is significantly decreased after treatment with GC-1 for 3 days (n=3). Significance (* p<0.05) was determined by a two-way ANOVA and Tukey's multiple comparisons test. FIG. 20B shows area under the curve (AUC) analysis of growth curve data. Significance (* p<0.05) was determined by t-test. FIG. 20C shows GC-1 repressed thyrosphere formation in SW-TRβ and SW-EV cells (n=4). Significance (* p<0.05) was determined by t-test. FIG. 20D shows an immunoblot that demonstrates that 5 days of GC-1 treatment induces apoptosis in SW-TRβ cells assessed by cleavage of PARP1 and caspase 3 (n=3).

FIGS. 21A-21C show GC-1 slows growth of parental ATC cells. FIG. 21A shows cell viability of unmodified ATC cell lines was measured by SRB assay after 4 days of treatment with 10 nM GC-1. Data are mean+/−SD; * p<0.05 determined by t-test; 3 independent experiments were performed per each treatment group. FIG. 21B shows relative cell growth of unmodified cell lines maintained in complete media was measured by cell counting after 8 days of treatment with either 10 nM GC-1 or 10 nM T₃. Data are mean+/−SD; * indicates p<0.05 determined by t-test. FIG. 21C shows the TRβ selective effect of GC-1 on expression levels of THRB, tumor suppressor, and MYC, tumor promoter, were determined by RT-QPCR in cells that were maintained in media supplemented with charcoal stripped serum. Data are mean+/−SD; * indicates p<0.05 determined by t-test; 2 independent experiments, n=3 per assay.

FIGS. 22A-22C show GC-1 increases efficacy of inhibitors. Cell viability was measured by SRB assay after 3 days of treatment with increasing concentrations Buparlisib or Alpelisib (FIG. 22A), Sorafenib (FIG. 22B), or Palbociclib (FIG. 22C) simultaneously in combination with 10 nM GC-1. * indicates p<0.05 determined by a two-way ANOVA and Sidaks multiple comparisons test; 3 independent experiments were performed per each treatment group.

FIGS. 23A-23E show GC-1 enhances the effects of inhibitors on cell migration. Cell migration was measured by scratch assay. Representative images demonstrate reduced scratch closure after two days when ATC cells are treated with GC-1 (FIG. 23A). Area under the curve (AUC) analysis shows SW1736 (FIG. 23B), 8505C (FIG. 23C), OCUT2 (FIG. 23D), and KTC-2 (FIG. 23E) cells all exhibit lower rates of scratch closer after treatment with GC-1 alone and further reduced scratch closure rates after combined treatment with GC-1 and inhibitors. * indicates p<0.05 determined by t-test.

FIGS. 24A-24C show GC-1 blocks thyrosphere outgrowth and increases the efficacy of therapeutic agents. Thyrosphere growth was determined for ATC cells after 3 days of treatment with 0.5 μM Buparlisib or Alpelisib (FIG. 24A), 10 μM Sorafenib (FIG. 24B), or 1 nM Palbociclib (FIG. 24C) under adherent culture conditions, followed by plating in conditions for spheroid growth in the presence of 10 nM GC-1 for 7 days. GC-1 alone and each therapeutic agent significantly blocked sphere formation in all cell types. GC-1 in addition to each therapeutic agent further inhibited or completely blocked sphere formation. * indicates p<0.05 determined by a two-way ANOVA and Sidaks multiple comparisons test; 3 independent experiments were performed per each treatment group; n=3 per analyses.

FIGS. 25A-25D show the baseline expression of thyroid hormone receptors in breast cells. FIG. 25A shows that THRB expression is significantly reduced in basal-like breast tumors in comparison to all other subtypes. THRA expression is significantly repressed in Basal-like breast cancers and greatest in HER2 expressing tumors as shown in FIG. 25B. Other significant differences are indicated in the figure. Both MCF10A and MDA-MB-231 express comparable THRA mRNA (n=4,), MCF10A expresses higher levels of THRB mRNA than MDA-MB-231 (n=4, p<0.05) as shown in FIG. 25C. FIG. 25D shows that MCF10A breast cancer cells express greater levels of TRβ protein and MDA-MB-231 breast cancer express greater levels of TRα protein as measured by immunoblot (n=6, p<0.05). Relative mRNA expression=target/GAPDH.

FIGS. 26A-26D show GC-1 increases efficacy of Alpelisib in Luminal A Breast Cancer.

FIG. 27 shows GC-1 increases efficacy of Alpelisib in HER2+ breast cancer.

FIG. 28 shows GC-1 affects cell migration of triple negative breast cancer cells.

FIGS. 29A-29D show GC-1 increases efficacy of inhibitors in triple negative breast cancer.

FIGS. 30A-30B show GC-1 blocks mammosphere outgrowth and increases the efficacy of therapeutic agents in triple negative breast cancer.

FIG. 31 shows results from GC-1 treatment in and in vivo xenograft model of anaplastic thyroid tumor.

### DETAILED DESCRIPTION

The present invention is directed to the use of thyroid hormone receptor beta-1 (TRβ) agonist as a therapy for early stage, aggressive, and treatment-resistant disease. In some aspects the TRβ agonist is used as a monotherapy. In some aspects the TRβ agonist is used as a neo-adjuvant or adjuvant therapy. As described herein, TRβ agonist treatment of cancer cells induces tumor suppressive transcriptome changes that induce or enhance cancer cell responsiveness to treatment with selective intracellular signaling inhibitors.

Accordingly, a first aspect of the present invention is directed to a combination therapy comprising a TRβ agonist and a primary cancer therapeutic.

As used herein, the term “combination therapy” refers to the administration of two or more therapeutic agents, i.e., one or more TRβ agonists in combination with a primary cancer therapeutic, suitable for the treatment of cancer, such as a solid malignant tumor. In some embodiments, the combination therapy is co-administered in a substantially simultaneous manner, such as in a single capsule or other delivery vehicle having a fixed ratio of active ingredients. In some embodiment, the combination therapy is administered in multiple capsules or delivery vehicles, each containing an active ingredient. In some embodiments, the therapeutic agents of the combination therapy are administered in a sequential manner, either at approximately the same time or at different times. For example, in one embodiment, the TRβ agonist is administered as a neo-adjuvant, i.e., it is administered prior to the administration of the primary cancer therapeutic. In other embodiments, the TRβ agonist is administered as a standard adjuvant therapy, i.e., it is administered after the administration of the primary cancer therapeutic. In all of the embodiments, the combination therapy provides beneficial effects of the drug combination in treating cancer, particularly in early stage, aggressive and treatment-resistant cancers as described herein.

In accordance with this and all aspects of the present invention, the combination therapy comprises a TRβ agonist. In one embodiment, the TRβ agonist is a selective TRβ agonist, exhibiting little or no binding to, or activity at, other thyroid receptor subtypes. In one embodiment, the TRβ agonist of the combination therapeutic does not bind to the TRα1 receptor. In one embodiment the TRβ agonist of the combination therapeutic does not bind to any of the TRα receptors, i.e., TRα1, TRα2, TRα3. In some embodiments, the TRβ agonist of the combination therapeutic does not bind to other TRβ receptor subtypes, i.e., TRβ2 or TRβ3.

Suitable TRβ agonists for inclusion in the combination therapy as described herein include those known in the art. These TRβ agonists include, without limitation, 3,5-Dimethyl-4(4′-hydroxy-3′-isopropylbenzyl) phenoxy) acetic acid (sobetirome; GC-1), 2-{4-[(3-benzyl-4-hydroxyphenyl)methyl]-3,5-dimethylphenoxy}acetic acid (GC-24), 2-[3,5-dichloro-4-[(6-oxo-5-propan-2-yl-1H-pyridazin-3-yl)oxy]phenyl]-3,5-dioxo-1,2,4-triazine-6-carbonitrile (MGL-3196; Resmetirom), 2-[4-(4-hydroxy-3-iodophenoxy)-3,5-diiodophenyl]acetic acid (tiratricol), (2S)-2-amino-3-[4-(4-hydroxy-3-iodophenoxy)-3,5-diiodophenyl]propanoic acid (triiodothyronine; T3), (2R, 4S)-4-(3-chlorophenyl)-2-[(3,5-dimethyl-4-(4′-hydroxy-3′-isopropylbenzyl)phenoxy) methyl]-2-oxido-[1-3]-dioxaphosphonane (Mb07811), (2R)-2-amino-3-[4-(4-hydroxy-3,5-diiodophenoxy)-3,5-diiodophenyl]propanoic acid (dextrothyroxine), 2-[3,5-dichloro-4-(4-hydroxy-3-propan-2-ylphenoxy)phenyl]acetic acid (KB-141), 3-[[3,5-dibromo-4-[4-hydroxy-3-(1-methylethyl)-phenoxy]-phenyl]-amino]-3-oxopropanoic acid (KB2115), (2S)-2-amino-3-[4-(4-hydroxy-3,5-diiodophenoxy)-3,5-diiodophenyl]propanoate (2S)-2-amino-3-[4-(4-hydroxy-3-iodophenoxy)-3,5-diiodophenyl]propanoate (Liotrix), (3,5-dimethyl-4-(4′-hydroxy-3′-isopropylbenzyl)phenoxy)methylphosphonic acid (MB07344). Derivatives and analogs of the aforementioned compounds having enhanced selectivity or agonist activity are also suitable for use in the combination therapeutic as described herein. As used herein, the term “a derivative thereof” refers to a salt thereof, a pharmaceutically acceptable salt thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, a geometric isomer thereof, a tautomer thereof, a mixture of tautomers thereof, an enantiomer thereof, a diastereomer thereof, a racemate thereof, a mixture of stereoisomers thereof, an isotope thereof (e.g., tritium, deuterium), or a combination thereof.

In one embodiment, the TRβ agonist of the combination therapy is 2-[4-[(4-hydroxy-3-propan-2-ylphenyl)methyl]-3,5-dimethylphenoxy]acetic acid, which is also known as sobetirome and GC-1 as disclosed in U.S. Pat. No. 5,883,294 to Scanlan et al., which is hereby incorporated by reference in its entirety. In other embodiments, the TRβ agonist is a GC-1 derivative as disclosed in U.S. Patent Application Publication No 2016/0244418 to Scanlan et al., which is hereby incorporated by reference in its entirety.

In some embodiments, the TRβ agonist of the combination therapy is 2-{4-[(3-benzyl-4-hydroxyphenyl)methyl]-3,5-dimethylphenoxy}acetic acid (GC-24), 2-[3,5-dichloro-4-[(6-oxo-5-propan-2-yl-1H-pyridazin-3-yl)oxy]phenyl]-3,5-dioxo-1,2,4-triazine-6-carbonitrile, which is also known as MGL-3196 and Resmetirom, or a derivative thereof, as disclosed in U.S. Pat. No. 7,807,674 to Haynes et al., which is hereby incorporated by reference in its entirety In other embodiments, the TRβ agonist is a prodrug of MGL-3196 as disclosed in U.S. Pat. No. 8,076,334 to Haynes, which is hereby incorporated by reference in its entirety.

The combination therapy as described herein further comprises a primary cancer therapeutic. The term “primary cancer therapeutic” refers to the initial treatment given to a patient based upon the diagnosis of cancer in the patient. The diagnosis of cancer may be the first occurrence of that disease in the patient, i.e., a newly diagnosed patient, or a reoccurrence of the disease in a patient, i.e., a relapsed patient. The primary cancer therapeutic is often part of a standard set of treatments. When used by itself, the primary cancer therapeutic, also known as first-line therapy, is the one accepted as the best treatment. If it does not cure the disease, slow disease progression, or if it causes severe side effects, other treatment may be added or used instead.

In one embodiment, the primary cancer therapeutic is a chemotherapeutic agent. Suitable chemotherapeutic agents include, for example and without limitation, paclitaxel, cisplatin, carboplatin, docetaxel, doxorubicin, and peplomycin (Gentile et al., “Preclinical and Clinical Combination Therapies in the Treatment of Anaplastic Thyroid Cancer,” Medical Oncology 37:19 (2020); De Leo et al. “Recent Advances in the Management of Anaplastic Thyroid Cancer,” Thyroid Research 13:17 (2020); Abe et al., “Anaplastic Thyroid Carcinoma: Current Issues in Genomics and Therapeutics,” Current Oncology Reports 23:31 (2021), which are hereby incorporated by reference in their entirety).

In another embodiment, the primary cancer therapeutic is an inhibitor of mTOR (mammalian target of rapamycin). A suitable mTOR inhibitor includes, for example and without limitation, everolimus (Abe et al., “Anaplastic Thyroid Carcinoma: Current Issues in Genomics and Therapeutics,” Current Oncology Reports 23:31 (2021), which is hereby incorporated by reference in its entirety).

In one embodiment, the primary cancer therapeutic is a ligand of PPARγ. Suitable ligands of PPARγ include, for example and without limitation, efatutazone, pioglitazone, and ciglitazone (De Leo et al. “Recent Advances in the Management of Anaplastic Thyroid Cancer,” Thyroid Research 13:17 (2020), which is hereby incorporated by reference in its entirety).

In one embodiment, the primary cancer therapeutic is a vascular disruptor molecule. A suitable vascular disruptor molecule includes, for example and without limitation, Fosbretabulin (De Leo et al. “Recent Advances in the Management of Anaplastic Thyroid Cancer,” Thyroid Research 13:17 (2020), which is hereby incorporated by reference in its entirety).

In another embodiment, the primary cancer therapeutic is an immune checkpoint inhibitor targeting PD-1 or PD-L1. Suitable inhibitors targeting PD-1 or PD-L1 include, for example and without limitation, pembrolizumab, spartalizumab, nivolumab, ipilimumab, and atezolizumab (De Leo et al. “Recent Advances in the Management of Anaplastic Thyroid Cancer,” Thyroid Research 13:17 (2020), which is hereby incorporated by reference in its entirety).

In one embodiment, the primary cancer therapeutic is an epigenetic modifier. Suitable epigenetic modifiers include, for example and without limitation, suberoylanilide hydroxamic acid (SAHA), PXD101, trichostatin A, romidepsin, JQ1, 5-aza-cdR, zebularine, and 5-aza-dC (Gentile et al., “Preclinical and Clinical Combination Therapies in the Treatment of Anaplastic Thyroid Cancer,” Medical Oncology 37:19 (2020); Zhu et al., “Epigenetic Modifications: Novel Therapeutic Approach for Thyroid Cancer,” Endocrinol Metab (Seoul) 32(3):326-331 (2017), which are hereby incorporated by reference in their entirety).

In one embodiment, the primary cancer therapeutic is an activator of the Interferon/JAK1/STAT1 signaling pathway. STAT1 exhibits tumor suppressor properties in a number of cancers, including colorectal cancer, hepatocellular carcinoma, esophageal cancer, pancreatic cancer, soft tissue sarcoma, and metastatic melanoma. Interferon gamma is a type II interferon that, when bound by cytokine, activates JAK1, which subsequently phosphorylates STAT1, leading to its dimerization, translocation to the nucleus, and activation of its transcription factor activity. Thus, in some embodiments, a suitable activator of the Interferon/JAK1/STAT1 pathway includes, for example and without limitation, a recombinant interferon gamma protein or polypeptides thereof (see e.g., Razaghi et al., “Review of the Recombinant Human Interferon Gamma as an Immunotherapeutic: Impacts of Production Platforms and Glycosylation,” J. Biotech. 240:48-60 (2016), which is hereby incorporated by reference in its entirety). In another embodiment, a suitable activator of the Interferon/JAK1/STAT1 pathway includes a recombinant interferon alpha protein (see e.g., Ningrum R., “Human Interferon Alpha-2b: A Therapeutic Protein for Cancer Treatment,” Scientifica 2014:970315 (2014), which is hereby incorporated by reference in its entirety. Suitable recombinant interferon alpha proteins include, without limitation Intron A® (Schering-Plough) and Roferon-A® (Hoffmann-LaRoche).

In another embodiment, a suitable activator of Interferon/JAK1/STAT1 signaling is recombinant oncostatin M protein (see Schaefer et al., “Activation of Stat3 and Stat1 DNA Binding and Transcriptional Activity in Human Brain Tumour Cell Line by gp130 Cytokine,” Cell Signal 12(3):143-151, which is hereby incorporated by reference in its entirety). A suitable recombinant oncostatin M protein has an amino acid sequence of SEQ ID NO: 1 as shown below or a polypeptide derived therefrom.

SEQ ID NO: 1
AAIGSCSKEYRVLLGQLQKQTDLMQDTSRLLDPYIRIQGLDVPKLREHCR
ERPGAFPSEETLRGLGRRGFLQTLNATLGCVLHRLADLEQRLPKAQDLER
SGLNIEDLEKLQMARPNILGLRNNIYCMAQLLDNSDTAEPTKAGRGASQP
PTPTPASDAFQRKLEGCRFLHGYHRFMHSVGRVFSKWGESPNRSR

In another embodiment, a suitable activator of the Interferon/JAK1/STAT1 signaling is a recombinant IL-6 protein. A suitable recombinant IL-6 protein has an amino acid sequence of SEQ ID NO: 2 or polypeptide derived thereof:

SEQ ID NO: 2
VPPGEDSKDVAAPHRQPLTSSERIDKQIRYILDGISALRKETCNKSNMCE
SSKEALAENNLNLPKMAEKDGCFQSGFNEETCLVKIITGLLEFEVYLEYL
QNRFESSEEQARAVQMSTKVLIQFLQKKAKNLDAITTPDPTTNASLLTKL
QAQNQWLQDMTTHLILRSFKEFLQSSLRALRQM

In another embodiment, the primary cancer therapeutic is an inhibitor of glycogen metabolism. Suitable inhibitors of glycogen metabolism include those known in the art (see, e.g., Zois et al. “Glycogen Metabolism has a Key Role in the Cancer Microenvironment and Provides New Targets for Cancer Therapy,” J. Mol. Med. 94:137-154 (2016), which is hereby incorporated by reference in its entirety). Exemplary modulators of glycogen metabolism include, for example and without limitation, metformin, lithium, valproate, sodium tungstate, and dichloroacetate.

Other suitable inhibitors of glycogen metabolism include inhibitors of glycogen phosphorylase, which is the key enzyme driving glycogenolysis. Glycogen phosphorylase inhibitors are classified by their site of action, e.g., catalytic active site inhibitors, nucleotide binding site (adenosine monophosphate (AMP) site inhibitors purine nucleotide site inhibitors, and the indole site inhibitors.

Suitable inhibitors of glycogen phosphorylase that work by inhibiting the catalytic active site include, without limitation glucose analogs, such as N-acetyl-β-D-glucosamine and glucopyranose spirohydantoin, and the azasugar 1,4-dideoxy-1,4-amino-D-arabinitol (DAB) (Andersen et al., “Inhibition of Glycogenolysis in Primary Rat Hepatocytes by 1, 4-dideoxy-1,4-imino-D-arabinitol,” Biochem J 342(Pt 3):545-50 (1999) and Jakobsen et al., “Iminosugars: Potential Inhibitors of Liver Glycogen Phosphorylase,” Bioorg Med Chem 9:733-44 (2001), which are hereby incorporated by reference in their entirety).

Exemplary glycogen phosphorylase inhibitors that bind to the AMP site, include, without limitation, isopropyl 4-(2-chlorophenyl)-1-ethyl-2-methyl-5-oxo-1,4,5,7-tetrahydro-furo[3,4-b]pyridine-3-carboxylate (BAY R3401), (4S)-1-ethyl-6-methyl-4-phenyl-5-propan-2-yloxycarbonyl-4H-pyridine-2,3-dicarboxylic acid (BAY-W1807). Exemplary glycogen phosphorylase inhibitors that function as indole carboxamide site inhibitors include, without limitation, 5-chloro-N-[(2S,3R)-4-(dimethylamino)-3-hydroxy-4-oxo-1-phenylbutan-2-yl]-1H-indole-2-carboxamide (CP-91149), 5-chloro-N-[(2S,3R)-4-[(3R,4S)-3,4-dihydroxypyrrolidin-1-yl]-3-hydroxy-4-oxo-1-phenylbutan-2-yl]-1H-indole-2-carboxamide (ingliforib), 5-chloro-N-[(2S)-3-(4-fluorophenyl)-1-(4-hydroxypiperidin-1-yl)-1-oxopropan-2-yl]-1H-indole-2-carboxamide (CP-320626), and 5-chloro-N-[3-(4-fluorophenyl)-1-(4-hydroxypiperidin-1-yl)-1-oxopropan-2-yl]-1H-indole-2-carboxamide (CP320626). Exemplary glycogen phosphorylase purine nucleoside site inhibitors suitable for use in the combination therapy as described herein include, without limitation, purines, flavopiridol, nucleosides, and olefin derivatives of flavopiridol.

Other glycogen phosphorylase inhibitors suitable for inclusion in the combination therapy of the present invention include, without limitation, (2R,3S)-2,3-bis[[(E)-3-(4-hydroxyphenyl)prop-2-enoyl]oxy]pentanedioic acid (FR258900), N-(3,5-dimethyl-benzoyl)-N′-((3-D-glucopyranosyl) urea (KB228), 5-chloro-N-[(2S,3R)-3-hydroxy-4-[methoxy(methyl)amino]-4-oxo-1-phenylbutan-2-yl]-1H-indole-2-carboxamide (CP-316819), 4-[3-(2-Chloro-4,5-Difluoro-Benzoyl)ureido]-3-Trifluoromethoxybenzoic Acid (AVE5688), GSK1362885, and PSN-357.

In one embodiment, the inhibitor of glycogen metabolism is an indole carboxamide site inhibitor of glycogen phosphorylase, e.g., 5-chloro-N-[(2S,3R)-4-(dimethylamino)-3-hydroxy-4-oxo-1-phenylbutan-2-yl]-1H-indole-2-carboxamide (CP-91149).

In some embodiments, the primary cancer therapeutic is a phosphoinositide 3-kinase (PI3K) inhibitor. Suitable PI3K inhibitors include those known in the art. Exemplary PI3K inhibitors include, without limitation, 5-(2,6-dimorpholin-4-ylpyrimidin-4-yl)-4-(trifluoromethyl)pyridin-2-amine (buparlisib), 4-morpholino-2-phenylquinazolines, pyrido[3′,2′:4,5]furo[3,2-d]pyrimidine, pyrido[3′,2′:4, 5]furo[3,2-d]pyrimidine, PWT-458 (pegylated-17-hydroxywortmannin), PX-866 (wortmannin analogue), 3-[6-(morpholin-4-yl)-8-oxa-3,5,10-triazatricyclo[7.4.0.0\hat{ }{2,7}]trideca-1(13),2,4,6,9,11-hexaen-4-yl]phenol (P1103), 5-[bis(morpholin-4-yl)-1,3,5-triazin-2-yl]-4-(trifluoromethyl)pyridin-2-amine (PQR-309), (S)-3-(1-((9H-purin-6-yl)amino)ethyl)-8-chloro-2-phenylisoquinolin-1(2H)-one (Duvelisib), N-[4-[[3-(3,5-dimethoxyanilino)quinoxalin-2-yl]sulfamoyl]phenyl]-3-methoxy-4-methylbenzamide (Voxtalisib), 1-[(3S)-3-[[6-[6-methoxy-5-(trifluoromethyl)pyridin-3-yl]-7,8-dihydro-5H-pyrido[4,3-d]pyrimidin-4-yl]amino]pyrrolidin-1-yl]propan-1-one (Leniolisib), (1,1-dimethylpiperidin-1-ium-4-yl) octadecyl phosphate(perifosine), 5-[7-methanesulfonyl-2-(morpholin-4-yl)-5H,6H,7H-pyrrolo[2,3-d]pyrimidin-4-yl]pyrimidin-2-amine (MEN1611), (2S)-1-N-[4-methyl-5-[2-(1,1,1-trifluoro-2-methylpropan-2-yl)pyridin-4-yl]-1,3-thiazol-2-yl]pyrrolidine-1,2-dicarboxamide (alpelisib), (1S,3S,4R)-4-[(3aS,4R,5S,7aS)-4-(aminomethyl)-7a-methyl-1-methylidene-3,3a,4,5,6,7-hexahydro-2H-inden-5-yl]-3-(hydroxymethyl)-4-methylcyclohexan-1-ol (rosiptor), 2-[6-(1H-indol-4-yl)-1H-indazol-4-yl]-5-[(4-propan-2-ylpiperazin-1-yl)methyl]-1,3-oxazole (nemiralisib), 2-amino-N-[7-methoxy-8-(3-morpholin-4-ylpropoxy)-2,3-dihydroimidazo[1,2-c]quinazolin-5-yl]pyrimidine-5-carboxamide (copanlisib), 2,4-difluoro-N-[2-methoxy-5-[4-methyl-8-[(3-methyloxetan-3-yl)methoxy]quinazolin-6-yl]pyridin-3-yl]benzenesulfonamide (Compound 5d), [6-(2-amino-1,3-benzoxazol-5-yl)imidazo[1,2-a]pyridin-3-yl]-morpholin-4-ylmethanone (serabelisib), 2-(morpholin-4-yl)-8-phenyl-4H-chromen-4-one (LY294002), 3-(3-fluorophenyl)-2-[(1S)-1-[(9H-purin-6-yl)amino]propyl]-4H-chromen-4-one (tenalisib), (2S)-2-[[2-[(4S)-4-(difluoromethyl)-2-oxo-1,3-oxazolidin-3-yl]-5,6-dihydroimidazo[1,2-d][1,4]benzoxazepin-9-yl]amino]propanamide (GDC-0077), 8-(6-methoxypyridin-3-yl)-3-methyl-1-[4-piperazin-1-yl-3-(trifluoromethyl)phenyl]imidazo[5,4-c]quinolin-2-one (BGT 226), [8-[6-amino-5-(trifluoromethyl)pyridin-3-yl]-1-[6-(2-cyanopropan-2-yl)pyridin-3-yl]-3-methylimidazo[4,5-c]quinolin-2-ylidene]cyanamide (panulisib), (5Z)-5-[(4-pyridin-4-ylquinolin-6-yl)methylidene]-1,3-thiazolidine-2,4-dione (GSK1059615), 7-methyl-2-morpholin-4-yl-9-[1-(phenylamino)ethyl]pyrido[2,1-b]pyrimidin-4-one (TGX 221), 4-[6-[[4-(cyclopropylmethyl)piperazin-1-yl]methyl]-2-(5-fluoro-1H-indol-4-yl)thieno[3,2-d]pyrimidin-4-yl]morpholine (PI 3065), 2-(difluoromethyl)-1-[4,6-di(morpholin-4-yl)-1,3,5-triazin-2-yl]benzimidazole (ZSTK474), 1-[4-[4-(dimethylamino)piperidine-1-carbonyl]phenyl]-3-[4-(4,6-dimorpholin-4-yl-1,3,5-triazin-2-yl)phenyl]urea (gedatolisib), 5-fluoro-3-phenyl-2-[(1S)-1-(7H-purin-6-ylamino)propyl]quinazolin-4-one (idelalisib), 3-[2,4-diamino-6-(3-hydroxyphenyl)pteridin-7-yl]phenol (TG-100-115), ethyl 6-[5-(benzenesulfonamido)pyridin-3-yl]imidazo[1,2-a]pyridine-3-carboxylate (HS-173), 2-[[2-methoxy-5-[[(E)-2-(2,4,6-trimethoxyphenyl)ethenyl]sulfonylmethyl]phenyl]amino]acetic acid (rigosertib), 2-(6,7-dimethoxyquinazolin-4-yl)-5-pyridin-2-yl-1,2,4-triazol-3-amine (CP-466722), N-[3-(2,1,3-benzothiadiazol-6-ylamino)quinoxalin-2-yl]-4-methylbenzenesulfonamide (pilaralisib), 2-[(1S)-1-[4-amino-3-(3-fluoro-4-propan-2-yloxyphenyl)pyrazolo[3,4-d]pyrimidin-1-yl]ethyl]-6-fluoro-3-(3-fluorophenyl)chromen-4-one (umbralisib), 5-(8-methyl-2-morpholin-4-yl-9-propan-2-ylpurin-6-yl)pyrimidin-2-amine (VS-5584), 2-methyl-2-[4-(3-methyl-2-oxo-8-quinolin-3-ylimidazo[4,5-c]quinolin-1-yl)phenyl]propanenitrile (dactolisib), 1-(4-{5-[5-amino-6-(5-tert-butyl-1,3,4-oxadiazol-2-yl)pyrazin-2-yl]-1-ethyl-1H-1,2,4-triazol-3-yl}piperidin-1-yl)-3-hydroxypropan-1-one (AZD8835), [(3aR,6E,9S,9aR,10R,11aS)-6-[(di(prop-2-enyl)amino)methylidene]-5-hydroxy-9-(methoxymethyl)-9a,11a-dimethyl-1,4,7-trioxo-2,3,3a,9,10,11-hexahydroindeno[7,6-h]isochromen-10-yl] acetate (sonolisib), 2-[[4-amino-3-(3-fluoro-5-hydroxyphenyl)pyrazolo[3,4-d]pyrimidin-1-yl]methyl]-5-[3-[2-(2-methoxyethoxy)ethoxy]prop-1-ynyl]-3-[[2-(trifluoromethyl)phenyl]methyl]quinazolin-4-one (RV-6153), 6-[2-[[4-amino-3-(3-hydroxyphenyl)pyrazolo[3,4-d]pyrimidin-1-yl]methyl]-3-[(2-chlorophenyl)methyl]-4-oxoquinazolin-5-yl]-N,N-bis(2-methoxyethyl)hex-5-ynamide (RV-1729), 2-(4-ethylpiperazin-1-yl)-N-[4-(2-morpholin-4-yl-4-oxochromen-8-yl)dibenzothiophen-1-yl]acetamide (KU-0060648), N′-cyclopropyl-N-quinolin-6-yl-7H-purine-2,6-diamine (puquitinib).

In some embodiments, the primary cancer therapeutic of the combination therapy as described herein is a PTEN activator. Suitable PTEN activators include those known in the art, see e.g., Boosani and Agrawal, “PTEN Modulators: A Patent Review,” Exp. Opin. Ther. Pat. 23(5):569-80 (2013), which is hereby incorporated by reference in its entirety. In one embodiment, the PTEN activator is an antibody. Suitable PTEN activator antibodies include, without limitation an anti-CD20 antibody (e.g., Ublituximab, Rituximab, and biosimilars thereof) (see e.g., Le Garff-Tavernier et al. “Analysis of CD16+CD56dim NK cells from CLL patients: evidence supporting a therapeutic strategy with optimized anti-CD20 monoclonal antibodies,” Leukemia 25 (1): 101-9 (2011), which is hereby incorporated by reference in its entirety), a HER2 antibody (e.g., Trastuzumab, Pertuzumab and biosimilars thereof) (see U.S. Patent Application Pub. No. 20020192211 to Hudziak and U.S. Pat. No. 6,399,063 to Hudziak, which are hereby incorporate by reference in their entirety), and an epidermal growth factor receptor antibody (e.g., Cetuximab) (see U.S. Pat. No. 7,060,808 to Goldstein, which is hereby incorporated by reference in its entirety).

In some embodiments, the PTEN activator is small molecule PTEN activator. Suitable small molecule activators of PTEN include, without limitation, N-[2-(diethylamino)ethyl]-5-{[(3Z)-5-fluoro-2-oxo-2,3-dihydro-1H-indol-3-ylidene]methyl}-2,4-dimethyl-1H-pyrrole-3-carboxamide N-[2-(diethylamino)ethyl]-5-{[(3Z)-5-fluoro-2-oxo-2,3-dihydro-1H-indol-3-ylidene]methyl}-2,4-dimethyl-1H-pyrrole-3-carboxamide (Sunitinib), N-(3-chloro-4-fluorophenyl)-7-methoxy-6-[3-(morpholin-4-yl)propoxy]quinazolin-4-amine (Gefitnib), N-(3-ethynylphenyl)-6,7-bis(2-methoxyethoxy)quinazolin-4-amine (Erlotinib), [(1S,3R,7S,8S,8aR)-8-[2-[(2R,4R)-4-hydroxy-6-oxooxan-2-yl]ethyl]-3,7-dimethyl-1,2,3,7,8,8a-hexahydronaphthalen-1-yl] 2,2-dimethylbutanoate (Simvastatin), [(1S,3R,7S,8S,8aR)-8-[2-[(2R,4R)-4-hydroxy-6-oxooxan-2-yl]ethyl]-3,7-dimethyl-1,2,3,7,8,8a-hexahydronaphthalen-1-yl] (2S)-2-methylbutanoate (Lovastatin), 5-[[4-[2-(methyl-pyridin-2-ylamino)ethoxy]phenyl]methyl]-1,3-thiazolidine-2,4-dione (Rosiglitazone), 7-[3-(azetidin-1-ylmethyl)cyclobutyl]-5-[3-(phenylmethoxy)phenyl]pyrrolo[3,2-e]pyrimidin-4-amine (NVP-AEW541), and (9 S,12S,14S,16S,17R)-8,13,14,17-tetramethoxy-4,10,12,16-tetramethyl-3,20,22-trioxo-2-azabicyclo[16.3.1]docosa-1(21),4,6,10,18-pentaen-9-yl carbamate (Herbimycin).

In some embodiments, the primary cancer therapeutic of the combination therapy described herein is an anti-estrogen therapeutic. Suitable anti-estrogen therapeutics include those known and used in the art. Exemplary anti-estrogen therapeutics include, without limitation, fulvestrant, tamoxifen, clomifene, raloxifene and toremifene. Aromatase inhibitors (e.g., Letrozole, Anastroxole, and Exemestane) that lower or reduce the production of estrogen are also suitable primary cancer therapeutics of the combination therapy in some embodiments.

In some embodiments, the primary therapeutic of the combination therapy disclosed herein is an inhibitor of the mitogen-activated protein kinase (MAPK) signaling pathway. The MAPK signaling pathway is a complex signaling pathway that is initiated by an extracellular stimulus in the form of growth factor(s) binding and activating receptor tyrosine kinases on the cell membrane. Downstream activation of RAS, RAF, and MEK proteins converge in the activation of ERK1/2 transcription factor activator. In some embodiments, the MAPK inhibitor is a RAS inhibitor, in particular, a KRAS inhibitor. In some embodiments, the MAPK inhibitor is a RAF inhibitor, in particular, a BRAF inhibitor. In some embodiments, the MAPK inhibitor is a MEK inhibitor. In some embodiments, the MAPK inhibitor is an ERK inhibitor.

MAPK inhibitors currently in use or in development for the treatment of cancer can be utilized as the primary therapeutic in the combination therapy as disclosed herein (see e.g., Braicu et al., “A Comprehensive Review on MAPK: A Promising Therapeutic Target in Cancer,” Cancers 11:1618 (2019), which is hereby incorporated by reference in its entirety. Non-limiting examples of MAPK inhibitors and their target of inhibition are summarized in Table 1 below.

TABLE 1
MAPK Inhibitors

| | | Target of |
|---|---|---|
| Tradename | IUPAC Name | Inhibition |
| AMG-510 | 6-fluoro-7-(2-fluoro-6-hydroxyphenyl)-1-(4- | KRAS |
| | methyl-2-propan-2-ylpyridin-3-yl)-4-[(2S)-2- | inhibitor |
| | methyl-4-prop-2-enoylpiperazin-1-yl]pyrido[2,3- | |
| | d]pyrimidin-2-one | |
| MRTX849 | 2-[(2S)-4-[7-(8-chloronaphthalen-1-yl)-2-[[(2S)-1- | KRAS |
| | methylpyrrolidin-2-yl]methoxy]-6,8-dihydro-5H- | inhibitor |
| | pyrido[3,4-d]pyrimidin-4-yl]-1-(2-fluoroprop-2- | |
| | enoyl)piperazin-2-yl]acetonitrile | |
| sorafenib | 4-[4-[[4-chloro-3- | BRAF |
| | (trifluoromethyl)phenyl]carbamoylamino] | inhibitor |
| | phenoxy]-N-methylpyridine-2-carboxamide | |
| vemurafenib | N-[3-[5-(4-chlorophenyl)-1H-pyrrolo[2,3- | BRAF |
| | b]pyridine-3-carbonyl]-2,4-difluorophenyl] | inhibitor |
| | propane-1-sulfonamide | |
| dabrafenib | N-[3-[5-(2-aminopyrimidin-4-yl)-2-tert-butyl- | BRAF |
| | 1,3-thiazol-4-yl]-2-fluorophenyl]-2,6- | inhibitor |
| | difluorobenzenesulfonamide | |
| selumetinib | 6-(4-bromo-2-chloroanilino)-7-fluoro-N-(2- | MEK |
| | hydroxyethoxy)-3-methylbenzimidazole-5- | inhibitor |
| | carboxamide | |
| trametinib | N-[3-[3-cyclopropyl-5-(2-fluoro-4-iodoanilino)- | MEK 1,2 |
| | 6,8-dimethyl-2,4,7-trioxopyrido[4,3-d]pyrimidin- | inhibitor |
| | 1-yl]phenyl]acetamide | |
| PD184352 | 2-(2-chloro-4-iodoanilino)-N- | MEK |
| | (cyclopropylmethoxy)-3,4-difluorobenzamide | inhibitor |
| Pimasertib | N-[(2S)-2,3-dihydroxypropyl]-3-(2-fluoro-4- | MEK 1,2 |
| | iodoanilino)pyridine-4-carboxamide | inhibitor |
| binimetinib | 6-(4-bromo-2-fluoroanilino)-7-fluoro-N-(2- | MEK 1,2 |
| | hydroxyethoxy)-3-methylbenzimidazole-5- | inhibitor |
| | carboxamide | |
| cobimetinib | [3,4-difluoro-2-(2-fluoro-4-iodoanilino)phenyl]- | MEK 1 |
| | [3-hydroxy-3-[(2S)-piperidin-2-yl]azetidin-1- | inhibitor |
| | yl]methanone | |
| ulixertinib | N-[(1S)-1-(3-chlorophenyl)-2-hydroxyethyl]-4- | ERK 1,2 |
| | [5-chloro-2-(propan-2-ylamino)pyridin-4-yl]-1H- | inhibitor |
| | pyrrole-2-carboxamide | |
| silymarin | (2R,3R)-3,5,7-trihydroxy-2-[(2R,3R)-3-(4- | ERK |
| | hydroxy-3-methoxyphenyl)-2-(hydroxymethyl)- | inhibitor |
| | 2,3-dihydro-1,4-benzodioxin-6-yl]- | |
| | 2,3-dihydrochromen-4-one | |

In some embodiments, the primary cancer therapeutic is an inhibitor of cancer stem cell formation. In some embodiments the inhibitor of cancer stem cell formation is a tyrosine kinase inhibitor. In some embodiments, the tyrosine kinase inhibitor is a vascular endothelial growth factor (VEGF) inhibitor. Suitable VEGF inhibitors include those known and used in the art, see e.g., those disclosed in Meadows ant Hurwitz, “Anti-VEGF Therapies in the Clinic,” Cold Spring Harb. Perspect. Med. 2:1006577 (2012) and De Leo et al., “Recent Advances in the Management of Anaplastic Thyroid Cancer,” Thyroid Research 13:17 (2020), which are hereby incorporated by reference in their entirety. In some embodiments, the VEGF inhibitor is a VEGF antibody. Suitable VEGF antibodies include, without limitation, bevacizumab (humanized anti-VEGF monoclonal antibody) and Ranibizumab (monoclonal VEGF-A antibody fragment (Fab)). In some embodiments, the VEGF inhibitor is a recombinant VEGF receptor, such as, for example, Aflibercept, a recombinant VEGF receptor fusion protein that binds VEGF A and B. In some embodiments, the VEGF inhibitor is a small molecule tyrosine kinase inhibitor. Suitable small molecule tyrosine kinase inhibitors include without limitation, the small molecule inhibitors listed in Table 2 below.

TABLE 2
Small Molecule Tyrosine Kinase Inhibitors

| Trade name | IUPAC name | Mode of Action |
|---|---|---|
| Sunitinib | N-[2-(diethylamino)ethyl]-5-[(Z)-(5-fluoro-2-oxo- | Inhibitor of VEGFs, |
| | 1H-indol-3-ylidene)methyl]-2,4-dimethyl-1H- | platelet derived growth |
| | pyrrole-3-carboxamide | factor (PDGF), c-kit, |
| | | Flt3, and Ret |
| Pazopanib | 5-[[4-[(2,3-dimethylindazol-6-yl)- | Inhibitor of VEGFRs, |
| | methylamino]pyrimidin-2-yl]amino]-2- | PDGFR and c-kit |
| | methylbenzenesulfonamide | |
| Axitinib | N-methyl-2-[[3-[(E)-2-pyridin-2-ylethenyl]-1H- | Inhibitor of VEGFs, |
| | indazol-6-yl]sulfanyl]benzamide | PDGF, and c-kit |
| Regorafenib | 4-[4-[[4-chloro-3- | inhibitor of VEGFRs, |
| | (trifluoromethyl)phenyl]carbamoylamino]-3- | PDGFR, c-kit, Raf, and |
| | fluorophenoxy]-N-methylpyridine-2-carboxamide | Ret |
| Sorafenib | 4-[4-[4-chloro-3- | Inhibitor of VEGFRs, |
| | (trifluoromethyl)phenyl]carbamoylamino]phenoxy]- | PDGFRs, c-kit, Ret, |
| | N-methylpyridine-2-carboxamide | and Raf |
| Brivanib | [(2R)-1-[4-[(4-fluoro-2-methyl-1H-indol-5-yl)oxy]- | inhibitor of VEGFR-2, |
| alaninate | 5-methylpyrrolo[2,1-f][1,2,4]triazin-6- | PDGFR, and Fibroblast |
| | yl]oxypropan-2-yl] (2S)-2-aminopropanoate | growth factor receptor |
| | | (FGFR) |
| Cediranib | 4-[(4-fluoro-2-methyl-1H-indol-5-yl)oxy]-6- | inhibitor of VEGFs, |
| | methoxy-7-(3-pyrrolidin-1-ylpropoxy)quinazoline | PDGF, and c-kit |
| Vandetanib | N-(4-bromo-2-fluorophenyl)-6-methoxy-7-[(1- | inhibitor of VEGFs, |
| | methylpiperidin-4-yl)methoxy]quinazolin-4-amine | PDGF, and epidermal |
| | | growth factor receptor |
| | | (EGFR), Ret |
| Linifinib | 1-[4-(3-amino-1H-indazol-4-yl)phenyl]-3- | inhibitor of VEGFs, |
| | (2-fluoro-5-methylphenyl)urea | PDGF, and c-kit |
| Lenvatinib | 4-[3-chloro-4- | inhibitor of VEGFs |
| | (cyclopropylcarbamoylamino)phenoxy]-7- | |
| | methoxyquinoline-6-carboxamide | |
| Nintedanib | methyl 2-hydroxy-3-N-[4-[methy]-[2-(4- | inhibitor of VEGFs, |
| | methylpiperazin-1-yl)acetyl]amino]phenyl]-C- | PDGFR, and FGFR |
| | phenylcarbonimidoyl]-1H-indole-6-carboxylate | |
| CLM94 | 4-chloro-N-(1,1,3-trioxo-2,3- | inhibitor of VEGFR |
| | dihydrobenzo[d]isothiazol-4-yl)benzamide | |
| CLM3 | pyrazolo[3,4-d]pyrimidine derivative | inhibitor of EGFR, Ret, |
| | | VEGFR |
| CLM24 | pyrazolopyrimidine derivative | inhibitor of EGFR, Ret, |
| | | VEGFR |
| CLM29 | pyrazolopyrimidine derivative | inhibitor of EGFR, Ret, |
| | | VEGFR |

In some embodiments, the tyrosine kinase inhibitor is a receptor tyrosine-protein kinase erbB-2 (also known as HER-2) inhibitor. In some embodiments, the HER2 inhibitor is a HER2 antibody. Suitable HER2 antibodies include, without limitation, the monoclonal antibodies Trastuzumab (Herceptin) and Pertuzumab (Perjeta). In some embodiments, the HER2 inhibitor is an antibody-drug conjugate, such as Ado-trastuxumab emtansine (Kadcyla or TDM-1) and Fam-trastuzumab deruxtecan (Enhertu), which are antibody-chemotherapeutic conjugates. In some embodiments, the HER2 inhibitor is a small molecule inhibitor, such as lapatinib and neratinib.

In some embodiments, the tyrosine kinase inhibitor is an inhibitor of the src family of kinases, including c-Src, Fyn, Yes, Lck, Lyn Hck, Fgr, and Blk, which have all been implicated in the pathogenesis of cancer. Exemplary src family of kinase inhibitors include, but are not limited to imatinib, dasatinib and nilotinib, saracatinib, and bosutinib

In some embodiments, the primary therapeutic of the combination therapy disclosed herein is a cyclin dependent kinase (CDK) inhibitor. In some embodiments, the CDK inhibitor is a pan-CDK inhibitor. In some embodiments, the CDK inhibitor is a CDK4/6 inhibitor. In some embodiments, the CDK inhibitor is an inhibitor of CDK2, CDK5, CDK7, CDK8, CDK9, CDK12, or combinations thereof. Exemplary CDK inhibitors for inclusion in the combination therapy are known in the art (see, e.g., Sanchez-Martinez et al., “Cyclin Dependent Kinase (CDK) Inhibitors as Anticancer Drugs: Recent Advances (2015-2019),” Bioorganic & Medicinal Chemistry Letters 29:126637 (2019), which is hereby incorporated by reference in its entirety) and summarized in Table 3 below.

TABLE 3
Small Molecule CDK Inhibitors

| | | Target |
|---|---|---|
| Tradename | IUPAC Name | CDK |
| palbociclib | 6-acetyl-8-cyclopentyl-5-methyl-2-[(5- | CDK4/6 |
| | piperazin-1-ylpyridin-2-yl)amino]pyrido[6,5- | |
| | d]pyrimidin-7-one | |
| ribociclib | 7-cyclopentyl-N,N-dimethyl-2-{[5-(piperazin- | CDK4/6 |
| | 1-yl)pyridin-2-yl]amino}-7H-pyrrolo[2,3- | |
| | d]pyrimidine-6-carboxamide | |
| Abemaciclib | N-[5-[(4-ethylpiperazin-1-yl)methyl]pyridin- | CDK4/6 |
| | 2-yl]-5-fluoro-4-(7-fluoro-2-methyl-3-propan- | |
| | 2-ylbenzimidazol-5-yl)pyrimidin-2-amine | |
| Trilaciclib | 2-[[5-(4-methylpiperazin-1-yl)pyridin-2- | CDK4/6 |
| | yl]amino]spiro[7,8- | |
| | dihydropyrazino[5,6]pyrrolo[1,2- | |
| | d]pyrimidine-9,1′-cyclohexane]-6-one | |
| SHR-6390 | | CDK4/6 |
| Milciclib | N,1,4,4-tetramethyl-8-[4-(4-methylpiperazin- | CDK2 |
| | 1-yl)anilino]-5H-pyrazolo[4,3-h]quinazoline- | |
| | 3-carboxamide | |
| BCD-115 | | CDK8/19 |
| MM-D37K | Synthetic peptide inhibitor | CDK4/6 |
| PF-06873600 | 6-(difluoromethyl)-8-[(1R,2R)-2-hydroxy-2- | CDK2/4/6 |
| | methylcyclopentyl]-2-[(1- | |
| | methylsulfonylpiperidin-4- | |
| | yl)amino]pyrido[2,3-d]pyrimidin-7-one | |
| Alvocidib | 2-(2-chlorophenyl)-5,7-dihydroxy-8-[(3S,4R)- | CDK9 |
| | 3-hydroxy-1-methylpiperidin-4-yl]chromen-4- | |
| | one | |
| Fostamatinib | [6-[[5-fluoro-2-(3,4,5- | |
| | trimethoxyanilino)pyrimidin-4-yl]amino]-2,2- | |
| | dimethyl-3-oxopyrido[3,2-b][1,4]oxazin-4- | |
| | yl]methyl dihydrogen phosphate | |
| Zotiraciclib | (16E)-14-methyl-20-oxa-5,7,14,27- | CDK9 |
| | tetrazatetracyclo[19.3.1.1^{2,6}.1^{8,12}]heptacosa- | |
| | 1(25),2(27),3,5,8,10,12(26),16,21,23-decaene | |
| C7001 | (3R,4R)-4-[[[7-(benzylamino)-3-propan-2- | CDK7 |
| (ICEC 0942) | ylpyrazolo[1,5-a]pyrimidin-5- | |
| | yl]amino]methyl]piperidin-3-ol | |
| BEY-1107 | | CDK1 |
| BPI-16350 | | CDK4/6 |
| FCN-437 | | CDK4/6 |
| CYC-065 | (2R,3S)-3-[[6-[(4,6-dimethylpyridin-3- | CDK2/7/9 |
| | yl)methylamino]-9-propan-2-ylpurin-2- | |
| | yl]amino]pentan-2-ol | |
| Seliciclib | (2R)-2-[[6-(benzylamino)-9-propan-2-ylpurin- | CDK2/9 |
| | 2-yl]amino]butan-1-ol | |
| AT-7519 | 4-[(2,6-dichlorobenzoyl)amino]-N-piperidin- | CDK9 |
| | 4-yl-1H-pyrazole-5-carboxamide | |
| Inditinib | 5-Nitro-5′-hydroxy-indirubin-3′-oxime | CDK2 |
| (AGM-130) | | |
| FN-1501 | N-[4-[(4-methylpiperazin-1- | CDK2 |
| | yl)methyl]phenyl]-4-(7H-pyrrolo[2,3- | |
| | d]pyrimidin-4-ylamino)-1H-pyrazole-5- | |
| | carboxamide | |
| SY-1365 | N-[(1S,3R)-3-[[5-chloro-4-(1H-indol-3- | CDK7 |
| | yl)pyrimidin-2-yl]amino]-1- | |
| | methylcyclohexy1]-5-[[(E)-4- | |
| | (dimethylamino)but-2-enoyl]amino]pyridine- | |
| | 2-carboxamide | |
| AZD-4573 | (1S,3R)-3-acetamido-N-[5-chloro-4-(5,5- | CDK9 |
| | dimethyl-4,6-dihydropyrrolo[1,2-b]pyrazol-3- | |
| | y1)pyridin-2-yl]cyclohexane-1-carboxamide | |
| TP-1287 | | CDK9 |
| Voruciclib | 2-[2-chloro-4-(trifluoromethyl)phenyl]-5,7- | CDK9 |
| | dihydroxy-8-[(2R,3S)-2-(hydroxymethyl)-1- | |
| | methylpyrrolidin-3-yl]chromen-4-one | |
| BAY- | 5-fluoro-4-(4-fluoro-2-methoxyphenyl)-N-[4- | CDK9 |
| 1251152 | [(methylsulfonimidoyl)methyl]pyridin-2- | |
| | yl]pyridin-2-amine | |
| Dinaciclib | 2-[(2S)-1-[3-ethyl-7-[(1-oxidopyridin-1-ium- | CDK12 |
| | 3-yl)methylamino]pyrazolo[1,5-a]pyrimidin- | |
| | 5-yl]piperidin-2-yl]ethanol | |

In some embodiments, the combination therapy as described herein provides a synergistic effect, as measured by, for example, the extent of the response, the response rate, the time to disease progression, or the survival period, as compared to the effect achievable on dosing with the primary therapeutic alone at its conventional dose. For example, the effect of the combination treatment is synergistic if a beneficial effect is obtained in a patient that does not respond (or responds poorly) to the primary therapeutic alone. In addition, the effect of the combination treatment is defined as affording a synergistic effect if the primary therapeutic is administered at dose lower than its conventional dose and the therapeutic effect, as measured by, for example, the extent of the response, the response rate, the time to disease progression or the survival period, is equivalent to that achievable on dosing conventional amounts of primary cancer therapeutic. In particular, synergy is deemed to be present if the conventional dose of the primary cancer therapeutic is reduced without detriment to one or more of the extent of the response, the response rate, the time to disease progression, and survival data, in particular without detriment to the duration of the response, but with fewer and/or less troublesome side-effects than those that occur when conventional doses of each component are used.

In one embodiment, the combination therapeutic encompasses a TRβ agonist and one or more primary cancer therapeutic formulated separately, but for administration together. In another embodiment, the combination therapeutic encompasses the TRβ agonist and one or more primary cancer therapeutic formulated together in a single formulation. A single formulation refers to a single carrier or vehicle formulated to deliver effective amounts of both therapeutic agents in a unit dose to a patient. The single vehicle is designed to deliver an effective amount of each of the agents, along with any pharmaceutically acceptable carriers or excipients. In some embodiments, the vehicle is a tablet, capsule, pill, or a patch. In other embodiments, the vehicle is a solution or a suspension. In yet another embodiment, the vehicle is a nanodelivery vehicle.

Suitable nanodelivery vehicles for the delivery of the TRβ agonist and one or more primary cancer therapeutics either together or separately, are known in the art and include, for example and without limitation, nanoparticles such as albumin particles (Hawkins et al., “Protein Nanoparticles as Drug Carriers in Clinical Medicine,” Advanced Drug Delivery Reviews 60(8): 876-885 (2008), which is hereby incorporated by reference in its entirety), cationic bovine serum albumin nanoparticles (Han et al., “Cationic Bovine Serum Albumin Based Self-assembled Nanoparticles as siRNA Delivery Vector for Treating Lung Metastasis Cancer,” Small 10(3): (2013), which is hereby incorporated by reference in its entirety), gelatin nanoparticles (Babaei et al., “Fabrication and Evaluation of Gelatine Nanoparticles for Delivering of Anti-cancer Drug,” Int'l J NanoSci. Nanotech. 4:23-29 (2008), which is hereby incorporated by reference in its entirety), gliadin nanoparticles (Gulfam et al., “Anticancer Drug-loaded Gliadin Nanoparticles Induced Apoptosis in Breast Cancer Cells,” Langmuir 28: 8216-8223 (2012), which is hereby incorporated by reference in its entirety), zein nanoparticles (Aswathy et al., “Biocompatible Fluorescent Zein Nanoparticles for Simultaneous Bioimaging and Drug Delivery Application,” Advances in Natural Sciences: Nanoscience and Nanotechnology 3(2) (2012), which is hereby incorporated by reference in its entirety), and casein nanoparticles (Elzoghby et al., “Ionically-crosslinked Milk Protein Nanoparticles as Flutamide Carriers for Effective Anticancer Activity in Prostate Cancer-Bearing Rats,” Eur. J. Pharm. Biopharm. 85(3): 444-451 (2013) which is hereby incorporated by reference in its entirety); liposomes (Feldman et al., “First-in-man Study of CPX-351: a Liposomal Carrier Containing Cytarabine and Daunorubicin in a Fixed 5:1 Molar Ratio for the Treatment of Relapsed and Refractory Acute Myeloid Leukemia,” J. Clin. Oncol. 29(8): 979-985 (2011); Ong et al., “Development of Stealth Liposome Coencapsulating Doxorubicin and Fluoxetine,” J. Liposome Res. 21(4): 261-271 (2011); and Sawant et al., “Palmitoyl Ascorbate-modified Liposomes as Nanoparticle Platform for Ascorbate-mediated Cytotoxicity and Paclitaxel Co-delivery,” Eur. J Pharm. Biopharm. 75(3): 321-326 (2010), which are hereby incorporated by reference in their entirety); polymeric nanoparticles, including synthetic polymers, such as poly-ε-caprolactone, polyacrylamine, and polyacrylate, and natural polymers, such as, e.g., albumin, gelatin, or chitosan (Agnihotri et al., “Novel Interpenetrating Network Chitosan-poly(ethylene oxide-g-acrylamide)hydrogel Microspheres for the Controlled Release of Capecitabine,” Int J Pharm 324: 103-115 (2006); Bilensoy et al., “Intravesical Cationic Nanoparticles of Chitosan and Polycaprolactone for the Delivery of Mitomycin C to Bladder Tumor,” Int J Pharm 371: 170-176 (2009), which are hereby incorporated by reference); dendrimer nanocarriers (e.g., poly(amido amide) (PAMAM)) (Han et al., “Peptide Conjugated PAMAM for Targeted Doxorubicin Delivery to Transferrin Receptor Overexpressed Tumors,” Mol Pharm 7: 2156-2165 (2010); and Singh et al., “Folate and Folate-PEG-PAMAM Dendrimers: Synthesis, Characterization, and Targeted Anticancer Drug Delivery Potential in Tumor Bearing Mice,” Bioconjugate Chem 19, 2239-2252 (2008), which are hereby incorporated by reference in their entirety); silica nanoparticle (e.g., xerogels and mesoporous silica nanoparticles) (He et al., “A pH-responsive Mesoporous Silica Nanoparticles Based Multi-drug Delivery System for Overcoming Multidrug Resistance,” Biomaterials 32: 7711-7720 (2011); Prokopowicz M., “Synthesis and In Vitro Characterization of Freeze-dried Doxorubicin-loaded Silica Xerogels,” J Sol-Gel Sci Technol 53: 525-533 (2010); Mayer et al., “Novel Hybrid Silica Xerogels for Stabilization and Controlled Release of Drug,” Int J Pharm 330:164-174 (2007), which are hereby incorporated by reference in their entirety).

The therapeutic agents and combination therapeutics for use in the methods described herein can be formulated into a pharmaceutical composition as any one or more of the active compounds described herein and a physiologically acceptable carrier (also referred to as a pharmaceutically acceptable carrier or solution or diluent). Such carriers and solutions include pharmaceutically acceptable salts and solvates of compounds used in the methods described herein, and mixtures comprising two or more of such compounds, pharmaceutically acceptable salts of the compounds and pharmaceutically acceptable solvates of the compounds. Such compositions are prepared in accordance with acceptable pharmaceutical procedures such as described in Remington: The Science and Practice of Pharmacy, 20th edition, ed. Alfonso R. Gennaro (2000), which is incorporated herein by reference in its entirety.

The term “pharmaceutically acceptable carrier” refers to a carrier that does not cause an allergic reaction or other untoward effect in patients to whom it is administered and are compatible with the other ingredients in the formulation. Pharmaceutically acceptable carriers include, for example, pharmaceutical diluents, excipients or carriers suitably selected with respect to the intended form of administration, and consistent with conventional pharmaceutical practices. For example, solid carriers/diluents include, but are not limited to, a gum, a starch (e.g., corn starch, pregelatinized starch), a sugar (e.g., lactose, mannitol, sucrose, dextrose), a cellulosic material (e.g., microcrystalline cellulose), an acrylate (e.g., polymethylacrylate), calcium carbonate, magnesium oxide, talc, or mixtures thereof. Pharmaceutically acceptable carriers may further comprise minor amounts of auxiliary substances such as wetting or emulsifying agents, preservatives or buffers, which enhance the shelf life or effectiveness of the therapeutic agent.

Reference to therapeutic agents described herein includes any analog, derivative, isomer, metabolite, pharmaceutically acceptable salt, pharmaceutical product, hydrate, N-oxide, crystal, polymorph, prodrug or any combination thereof. In certain embodiments, the therapeutic agents disclosed herein may be in a prodrug form, meaning that it must undergo some alteration (e.g., oxidation or hydrolysis) to achieve its active form.

The therapeutic agents in a free form can be converted into a salt, if need be, by conventional methods. The term “salt” used herein is not limited as long as the salt is pharmacologically acceptable; preferred examples of salts include a hydrohalide salt (for instance, hydrochloride, hydrobromide, hydroiodide and the like), an inorganic acid salt (for instance, sulfate, nitrate, perchlorate, phosphate, carbonate, bicarbonate and the like), an organic carboxylate salt (for instance, acetate salt, maleate salt, tartrate salt, fumarate salt, citrate salt and the like), an organic sulfonate salt (for instance, methanesulfonate salt, ethanesulfonate salt, benzenesulfonate salt, toluenesulfonate salt, camphorsulfonate salt and the like), an amino acid salt (for instance, aspartate salt, glutamate salt and the like), a quaternary ammonium salt, an alkaline metal salt (for instance, sodium salt, potassium salt and the like), an alkaline earth metal salt (magnesium salt, calcium salt and the like) and the like. In addition, hydrochloride salt, sulfate salt, methanesulfonate salt, acetate salt and the like are preferred as “pharmacologically acceptable salt” of the compounds disclosed herein.

Another aspect of the present invention is directed to a method of inducing differentiation in a population of cancer cells. This method involves administering to a population of cancer cells having a decreased level of TRβ expression or activity relative to a corresponding population of non-cancer cells of similar origin, a thyroid hormone receptor beta-1 (TRβ) agonist in an amount effective to induce differentiation of said cancer cells of the population.

A related aspect of the present invention is directed to a method of treating cancer in a subject. This method involves administering to a subject having cancer, wherein the cancer is characterized by cells having a decreased level of TRβ expression or decreased level of TRβ activity relative to corresponding non-cancer cells of similar origin, a thyroid hormone receptor beta-1 (TRβ) agonist in an amount effective to treat the cancer.

A related aspect of the present invention is directed to a method of treating an advanced form of thyroid cancer such as advanced anaplastic thyroid cancer, poorly differentiated thyroid cancer, metastatic thyroid cancer, treatment resistant thyroid cancer, or recurrent thyroid cancer, or an advanced form of breast cancer such as stage four breast cancer or triple negative breast cancer. This method involves administering to a patient having the advanced form of thyroid or breast cancer, a thyroid hormone receptor beta-1 (TRβ) agonist in an amount effective to treat the advanced cancer.

As described herein, the inventors have found that administration of a TRβ agonist to cancer cells having decreased levels of TRβ expression or activity relative to corresponding non-cancer cells of similar origin or to cancer cells of an advanced form of cancer induces tumor suppressive transcriptomic changes, including but not limited to increased JAK1-STAT1 (interferon) signaling, PI3K phosphatase activity, and decreased glycogen signaling (novel cancer metabolic pathway). These changes inhibit oncogenic signaling, induce apoptotic signaling, and induce a more differentiated cancer cell state. In addition, administration of the TRβ agonist induces a less aggressive phenotype of the cancer cells by increasing the cell doubling time, decreasing cell growth, decreasing cell migration, increasing apoptosis, inducing cell senescence and cell cycle arrest, reducing stem-cell characteristics and inducing more differentiated cell characteristic, and inducing or increasing cancer cell sensitivity to primary cancer therapeutic treatment.

In accordance with the methods described herein a “subject” refers to any animal having cancer, where the cancer is characterized by cells having a decreased level of TRβ expression or activity relative to corresponding non-cancer cells of similar origin. In some embodiments, the subject is any animal having an advance form of thyroid cancer (e.g., advanced anaplastic thyroid cancer, poorly differentiated thyroid cancer, metastatic thyroid cancer, treatment resistant thyroid cancer, or recurrent thyroid cancer) or an advanced form of breast cancer (e.g., triple negative breast cancer or stage four breast cancer). In one embodiment, the subject is a mammal. Exemplary mammalian subjects include, without limitation, humans, non-human primates, dogs, cats, rodents (e.g., mouse, rat, guinea pig), horses, cattle and cows, sheep, and pigs.

In accordance with these methods described herein, the cancer or cancer cells to be treated include any malignant solid tumor or tumor cells, where the cells of the tumor exhibit a reduced level of TRβ expression or activity relative to corresponding non-cancer cells of similar origin. Suitable cancers and cancer cells to be treated in accordance with the methods described herein include, without limitation, breast cancer and breast cancer cells, thyroid cancer and thyroid cancer cells, bladder cancer and bladder cancer cells, cervical cancer and cervical cancer cells, colorectal cancer and colorectal cancer cells, esophageal cancer and esophageal cancer cells, gastric cancer and gastric cancer cells, head and neck cancer and head and neck cancer cells, kidney cancer and kidney cancer cells, liver cancer and liver cancer cells, lung cancer and lung cancer cells, nasopharyngeal cancer and nasopharyngeal cancer cells, ovarian cancer and ovarian cancer cells, cholangiocarcinoma and cholangiocarcinoma cells, pancreatic cancer and pancreatic cancer cells, prostate cancer and prostate cancer cells, glioblastoma and glioblastoma cells, astrocytoma and astrocytoma cells, melanoma and melanoma cells, mesothelioma, musculoskeletal sarcoma, and soft tissue sarcoma.

Tumors suitable for treatment in accordance with the methods of the present invention are those where the cells of the tumor exhibit reduced levels of TRβ expression or activity relative to corresponding non-cancer cells of similar origin. TRβ “expression levels” encompass the production of any product by the THRB gene including but not limited to transcription of mRNA and translation of polypeptides, peptides, and peptide fragments. Measuring or detecting expression levels encompasses assaying, measuring, quantifying, scoring, or detecting the amount, concentration, or relative abundance of a gene product. It is recognized that methods of assaying TRβ expression include direct measurements and indirect measurements. One skilled in the art is capable of selecting an appropriate method of evaluating TRβ expression.

In one embodiment, TRβ expression levels are measured using a nucleic acid detection assay. In one embodiment, the DNA levels are measure. In another embodiment, RNA, e.g., mRNA, levels are measured. RNA is preferably reverse-transcribed to synthesize complementary DNA (cDNA), which is then amplified and detected or directly detected. The detected cDNA is measured and the levels of cDNA serve as an indicator of the RNA or mRNA levels present in a sample. Reverse transcription may be performed alone or in combination with an amplification step, e.g., reverse transcription polymerase chain reaction (RT-PCR), which may be further modified to be quantitative, e.g., quantitative RT-PCR as described in U.S. Pat. No. 5,639,606, which is hereby incorporated by reference in its entirety.

In one embodiment, the extracted nucleic acids, including DNA and/or RNA, are analyzed directly without an amplification step. Direct analysis may be performed with different methods including, but not limited to, nanostring technology (Geiss et al. “Direct Multiplexed Measurement of Gene Expression with Color-Coded Probe Pairs,” Nat Biotechnol 26(3): 317-25 (2008), which is hereby incorporated by reference in its entirety). In another embodiment, direct analysis can be performed using immunohistochemical techniques.

In other embodiments, it may be beneficial or otherwise desirable to amplify the cancer cell extracted nucleic acids prior to detection/analysis. Methods of nucleic acid amplification, including quantitative amplification, are commonly used and generally known in the art. Quantitative amplification will allow quantitative determination of relative amounts of the THRB nucleic acids. Nucleic acid amplification methods include, without limitation, polymerase chain reaction (PCR) (U.S. Pat. No. 5,219,727, which is hereby incorporated by reference in its entirety) and its variants such as in situ polymerase chain reaction (U.S. Pat. No. 5,538,871, which is hereby incorporated by reference in its entirety), quantitative polymerase chain reaction (U.S. Pat. No. 5,219,727, which is hereby incorporated by reference in its entirety), nested polymerase chain reaction (U.S. Pat. No. 5,556,773), self-sustained sequence replication and its variants (Guatelli et al. “Isothermal, In vitro Amplification of Nucleic Acids by a Multienzyme Reaction Modeled after Retroviral Replication,” Proc Natl Acad Sci USA 87(5): 1874-8 (1990), which is hereby incorporated by reference in its entirety), transcriptional amplification and its variants (Kwoh et al. “Transcription-based Amplification System and Detection of Amplified Human Immunodeficiency Virus type 1 with a Bead-Based Sandwich Hybridization Format,” Proc Natl Acad Sci USA 86(4): 1173-7 (1989), which is hereby incorporated by reference in its entirety), Qb Replicase and its variants (Miele et al. “Autocatalytic Replication of a Recombinant RNA.” J Mol Biol 171(3): 281-95 (1983), which is hereby incorporated by reference in its entirety), cold-PCR (Li et al. “Replacing PCR with COLD-PCR Enriches Variant DNA Sequences and Redefines the Sensitivity of Genetic Testing.” Nat Med 14(5): 579-84 (2008), which is hereby incorporated by reference in its entirety) or any other nucleic acid amplification method known in the art. Depending on the amplification technique that is employed, the amplified molecules are detected during amplification (e.g., real-time PCR) or subsequent to amplification using detection techniques known to those of skill in the art. Suitable nucleic acid detection assays include, for example and without limitation, northern blot, microarray, serial analysis of gene expression (SAGE), next-generation RNA sequencing (e.g., deep sequencing, whole transcriptome sequencing, exome sequencing), gene expression analysis by massively parallel signature sequencing (MPSS), immune-derived colorimetric assays, and mass spectrometry (MS) methods (e.g., MassARRAY® System).

In another embodiment, TRβ protein levels are measured in the cancer cells. TRβ protein levels can be measured using an immunoassay. Generally, an immunoassay involves contacting the cancer cell sample from the subject a binding reagents, e.g., an antibody, that binds specifically to the TRβ. The binding reagent is coupled to a detectable label, either directly or indirectly. For example, an antibody can be directly coupled to a detectable label or indirectly coupled to a detectable label via a secondary antibody. The one or more labeled binding reagents bound to TRβ (i.e., a binding reagent-marker complex) in the sample is detected, and the amount of labeled binding reagent that is detected serves as an indicator of the amount or expression level of TRβ in the sample. Immunoassays that are well known in the art and suitable for measuring TRβ include, for example and without limitation, an immunohistochemical assay, radioimmunoassay, enzyme linked immunosorbent assay (ELISA), immunoradiometric assay, gel diffusion precipitation reaction, immunodiffusion assay, in situ immunoassay, western blot, precipitation reaction, complement fixation assay, immunofluorescence assay, and immunoelectrophoresis assay.

In another embodiment, TRβ protein levels are measured using one-dimensional and two-dimensional electrophoretic gel analysis, high performance liquid chromatography (HPLC), reverse phase HPLC, Fast protein liquid chromatograph (FPLC), mass spectrometry (MS), tandem mass spectrometry, liquid crystal-MS (LC-MS) surface enhanced laser desorption/ionization (SELDI), MALDI, and/or protein sequencing.

In accordance with the methods of the present invention, the expression levels of TRβ is compared to TRβ expression levels in non-cancer cells of similar origin, i.e., “control” cells to determine whether the cancer cells will be responsive to the therapeutic treatment and methods described herein. In one embodiment, the control expression level of TRβ is the average TRβ expression level of a cell corresponding to the cancerous cell type in a cohort of healthy individuals. In another embodiment, the control TRβ expression level is the average TRβ expression level in a cell sample taken from the subject to be treated, but at an earlier time point (e.g., a pre-cancerous time point). In all of these embodiments, a decrease in the TRβ expression level in the cancer cells from the subject relative to the control cell expression level identifies the cancer as one suitable for treatment in accordance with the methods described herein.

A “decreased expression level” refers to an expression level (i.e., protein or gene expression level) that is lower than the control level. For example, a decreased expression level is at least 10%, at least 20%, at least 30%, at least 40%, at least 50%, at least 80%, at least 90%, at least 2-fold, at least 3-fold, at least 5-fold, at least 10-fold, at least 50-fold or at least 100-fold lower than the control expression level.

In some embodiments, the methods described herein are used to induce differentiation of thyroid cancer cells for the treatment of thyroid cancer. In some embodiments, the thyroid cancer is advanced anaplastic thyroid cancer, poorly differentiated thyroid cancer, metastatic thyroid cancer, treatment resistant thyroid cancer, or recurrent thyroid cancer.

In some embodiments, the methods described herein are used to induce differentiation in breast cancer cells for the treatment of breast cancer. In some embodiments, the breast cancer is triple negative breast cancer, estrogen receptor-positive breast cancer, metastatic breast cancer, HER2 positive breast cancer, and variants thereof.

In one embodiment the cancer or cells of the cancer are resistant to primary cancer therapeutic treatment prior to administering the TRβ agonist, and administering the TRβ agonist is carried out in an amount effective to re-sensitize the cancer cells to primary cancer therapeutic treatment.

In another embodiment, the cancer or cells of the cancer are not resistant to primary cancer therapeutic treatment, and the TRβ agonist is administered in an amount effective to inhibit, slow, or prevent cancer cell resistance to primary cancer therapeutic treatment.

In some embodiments, the method of treating cancer as described herein further involves administering a primary cancer therapeutic in conjunction with the TRβ agonist. In some embodiments, the TRβ agonist and said primary cancer therapeutic are administered concurrently. In some embodiments, the TRβ agonist said primary cancer therapeutic are administered sequentially. In some embodiments, the TRβ agonist is administered prior to administering said primary cancer therapeutic. In some embodiments, the TRβ agonist is administered after the primary cancer therapeutic is administered. Exemplary TRβ agonist and primary cancer therapeutics are described supra.

In accordance with the methods described herein, administration of the TRβ agonist alone or in combination with one or more primary cancer therapeutics is carried out by systemic or local administration. Suitable modes of systemic administration of the therapeutic agents and/or combination therapeutics disclosed herein include, without limitation, orally, topically, transdermally, parenterally, intradermally, intrapulmonary, intramuscularly, intraperitoneally, intravenously, subcutaneously, or by intranasal instillation, by intracavitary or intravesical instillation, intraocularly, intra-arterially, intralesionally, or by application to mucous membranes. In certain embodiments, the therapeutic agents of the methods described herein are delivered orally. Suitable modes of local administration of the therapeutic agents and/or combinations disclosed herein include, without limitation, catheterization, implantation, direct injection, dermal/transdermal application, or portal vein administration to relevant tissues, or by any other local administration technique, method or procedure generally known in the art. The mode of affecting delivery of agent will vary depending on the type of therapeutic agent and the type of prostate cancer to be treated.

A therapeutically effective amount of the TRβ agonist alone or in combination with the primary cancer therapeutic in the methods disclosed herein is an amount that, when administered over a particular time interval, results in achievement of one or more therapeutic benchmarks (e.g., slowing or halting of tumor growth, tumor regression, cessation of symptoms, etc.). The TRβ agonist alone or in combination with the primary cancer therapeutic for use in the presently disclosed methods may be administered to a subject one time or multiple times. In those embodiments where the compounds are administered multiple times, they may be administered at a set interval, e.g., daily, every other day, weekly, or monthly. Alternatively, they can be administered at an irregular interval, for example on an as-needed basis based on symptoms, patient health, and the like. For example, a therapeutically effective amount may be administered once a day (q.d.) for one day, at least 2 days, at least 3 days, at least 4 days, at least 5 days, at least 6 days, at least 7 days, at least 10 days, or at least 15 days. Optionally, the status of the cancer or the regression of the cancer is monitored during or after the treatment, for example, by a multiparametric ultrasound (mpUS), multiparametric magnetic resonance imaging (mpMRI), and nuclear imaging (positron emission tomography [PET]) of the subject. The dosage of the therapeutic agent(s) or combination therapy administered to the subject can be increased or decreased depending on the status of the cancer or the regression of the cancer detected.

The skilled artisan can readily determine this amount, on either an individual subject basis (e.g., the amount of a compound necessary to achieve a particular therapeutic benchmark in the subject being treated) or a population basis (e.g., the amount of a compound necessary to achieve a particular therapeutic benchmark in the average subject from a given population). Ideally, the therapeutically effective amount does not exceed the maximum tolerated dosage at which 50% or more of treated subjects experience side effects that prevent further drug administrations.

A therapeutically effective amount may vary for a subject depending on a variety of factors, including variety and extent of the symptoms, sex, age, body weight, or general health of the subject, administration mode and salt or solvate type, variation in susceptibility to the drug, the specific type of the disease, and the like.

The effectiveness of the methods of the present application in inducing differentiation of cancer cells and/or treating cancer may be evaluated, for example, by assessing changes in tumor burden and/or disease progression following treatment with the TRβ agonist alone or in combination with the one or more primary cancer therapeutics as described herein according to the Response Evaluation Criteria in Solid Tumours (Eisenhauer et al., “New Response Evaluation Criteria in Solid Tumours: Revised RECIST Guideline (Version 1.1),” Eur. J. Cancer 45(2): 228-247 (2009), which is hereby incorporated by reference in its entirety). In some embodiments, tumor burden and/or disease progression is evaluated using imaging techniques including, e.g., X-ray, computed tomography (CT) scan, magnetic resonance imaging, multiparametric ultrasound (mpUS), multiparametric magnetic resonance imaging (mpMRI), and nuclear imaging (positron emission tomography [PET]) (Eisenhauer et al., “New Response Evaluation Criteria in Solid Tumours: Revised RECIST Guideline (Version 1.1),” Eur. J. Cancer 45(2): 228-247 (2009), which is hereby incorporated by reference in its entirety). Cancer regression or progression may be monitored prior to, during, and/or following treatment with one or more of the therapeutic agents described herein.

In some embodiments, the therapeutically effective amount of the TRβ agonist is the amount that results in a reduction of the effective dose of the primary therapeutic drug. In another words, the combination of TRβ agonist and primary cancer therapeutic allows for a reduced dosing level of the primary cancer therapeutic as compared to when the primary therapeutic is administered as a monotherapy. In some embodiments, the dose of the primary cancer therapeutic is reduced by 1%, 2.5%, 5%, 7.5%, 10%, 12.5%, 15%, 17.5%, 20%, 22.5%, 25%, 27.5%, 30%, 32.5%, 35%, 37.5%, 40%, 42.5%, 45%, 47.5%, 50% when administered in combination with a TRβ agonist. In some embodiments, the dose of the primary cancer therapeutic is reduced by more than 50% when administered in combination with the TRβ agonist. In some embodiments, administering the TRβ agonist in combination with the primary cancer therapeutic lowers the dose of the primary cancer therapeutic to a dose having reduced toxicity and/or side-effects as compared to the monotherapeutic dose of the primary therapeutic. Thus, in some embodiments, administering the TRβ agonist in combination with a lower dose of primary cancer therapeutic relative to a monotherapeutic dose of the primary therapeutic results in a reduction in toxicity to the subject and/or a reduction in primary therapeutic related side effects.

In some embodiments, the response to treatment with the methods described herein results in at least about 1%, 2%, 3%, 4%, 5%, 6%, 7%, 8%, 9%, 10%, 20%, 30%, 40%, 50%, 60%, 70%, 80%, 90%, 95%, 99%, or 100% decrease in tumor size as compared to baseline tumor size. Thus, the response to treatment with any of the methods described herein may be partial (e.g., at least a 30% decrease in tumor size, as compared to baseline tumor size) or complete (elimination of the tumor).

In some embodiments, the effectiveness of the methods described herein may be evaluated, for example, by assessing drug induced cancer cell differentiation following treatment with the TRβ agonist alone or in combination with the one or more primary cancer therapeutics.

In some embodiments, the methods described herein may be effective to inhibit disease progression, inhibit tumor growth, reduce primary tumor size, relieve tumor-related symptoms, inhibit tumor-secreted factors (e.g., tumor-secreted hormones), delay the appearance of primary or secondary cancer tumors, slow development of primary or secondary cancer tumors, decrease the occurrence of primary or secondary cancer tumors, slow or decrease the severity of secondary effects of disease, arrest tumor growth, and/or achieve regression of cancer in a selected subject. Thus, the methods described herein are effective to increase the therapeutic benefit to the selected subject.

In some embodiments, the methods described herein reduce the rate of tumor growth in the selected subject by at least about 1%, 2%, 3%, 4%, 5%, 6%, 7%, 8%, 9%, 10%, 20%, 30%, 40%, 50%, 60%, 70%, 80%, 90%, 95%, 99%, or more. In certain embodiments, the methods described herein reduce the rate of tumor invasiveness in the selected subject by at least about 1%, 2%, 3%, 4%, 5%, 6%, 7%, 8%, 9%, 10%, 20%, 30%, 40%, 50%, 60%, 70%, 80%, 90%, 95%, 99%, or more. In specific embodiments, the methods described herein reduce the rate of tumor progression in the selected subject by at least about 1%, 2%, 3%, 4%, 5%, 6%, 7%, 8%, 9%, 10%, 20%, 30%, 40%, 50%, 60%, 70%, 80%, 90%, 95%, 99%, or more. In various embodiments, the methods described herein reduce the rate of tumor recurrence in the selected subject by at least about 1%, 2%, 3%, 4%, 5%, 6%, 7%, 8%, 9%, 10%, 20%, 30%, 40%, 50%, 60%, 70%, 80%, 90%, 95%, 99%, or more. In some embodiments, the methods described herein reduce the rate of metastasis in the selected subject by at least about 1%, 2%, 3%, 4%, 5%, 6%, 7%, 8%, 9%, 10%, 20%, 30%, 40%, 50%, 60%, 70%, 80%, 90%, 95%, 99%, or more.

In some embodiments, the methods described herein reduce or inhibit metastases in the selected subject.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

### EXAMPLES

### Materials and Methods for Examples 1-5

Culture of thyroid cell lines. Anaplastic thyroid cancer cell lines (SW1736 and KTC-2) were cultured in RPMI 1640 growth media with L-glutamine (300 mg/L), sodium pyruvate and nonessential amino acids (1%) (Cellgro/Mediatech), supplemented with 10% fetal bovine serum (Life Technologies) and penicillin-streptomycin (200 IU/L) (Cellgro/Mediatech) at 37° C., 5% CO₂, and 100% humidity. The cell lines were generously provided by Dr. John Copland III (Mayo Clinic). Lentivirally modified SW1736 cells were generated as recently described (Gillis et al., “Thyroid Hormone Receptor β Suppression of RUNX2 is Mediated by Brahma Related Gene 1 Dependent Chromatin Remodeling.” Endocrinol. 159(6):2484-94 (2018), which is hereby incorporated by reference in its entirety) with either an empty vector (SW-EV) or to overexpress TRβ (SW-TRβ). SW-EV and SW-TRβ were grown in the above conditions with the addition of 1 μg/ml puromycin (Gold Bio). All cells were authenticated by the Vermont Integrative Genomics Resource at the University of Vermont using short tandem repeat profiles and Promega GenePrint10 System (SW1736, May 2019; KTC-2, October 2019).

RNA-seq Library Construction and Quality Control. 80% confluent monolayers of SW-EV and SW-TRβ cells were hormone starved for 24 hours in phenol red free RPMI with charcoal-stripped fetal bovine serum. 10⁻⁸M T₃ was added and incubated for 24 hours prior to sample collection. Total RNA was extracted and purified using RNeasy Plus Kit (Qiagen) according to manufacturer's protocol. Purity of the total RNA samples was assessed via BioAnalyzer (Agilent) and samples with a RIN score >7 were used for library construction. rRNA was depleted using 1 μg of total RNA with the RiboErase kit (KAPA Biosystems). Strand-specific Illumina cDNA libraries were prepared using the KAPA Stranded RNA-Seq library preparation kit with 10 cycles of PCR (KAPA Biosystems). Library quality was assessed by BioAnalyzer (Agilent) to ensure an average library size of 300 bp and the absence of excess adaptors in each sample. RNA-Seq libraries were pooled and sequenced on the Illumina HiSeq 1500 with 50 bp single-end reads. Quality scores across sequenced reads were assessed using FASTQC. All samples were high quality. For alignment and transcript assembly, the sequencing reads were mapped to hg38 using STAR. Sorted reads were counted using HTSeq and differential expression analysis was performed using DESeq2. Genes with a p-value of <0.05 and a fold change of >2 were considered differentially expressed and were used for further analysis through Ingenuity Pathway Analysis (IPA) (Qiagen) and Gene Set Enrichment Analysis (GSEA). Table 6 herein shows the Ingenuity Pathways that were assessed (first column of Table 6) and genes within that pathway that were differentially expressed (last column of Table 6 labeled “Molecules”). The column labeled “−log(p-value)” indicates the fold increase in the identified gene expression pathway in SW-TRβ cells relative to SW-EV cells.

Immunoblot Analysis. Proteins were isolated from whole cells in lysis buffer (20 mM Tris-HCl [pH 8], 137 mM NaCl, 10% glycerol, 1% Triton X-100, and 2 mM EDTA) containing Protease Inhibitor Cocktail 78410 (Thermo Scientific). Proteins were resolved by polyacrylamide gel electrophoresis on 10% sodium dodecyl sulfate gels EC60752 (Life Technologies) and immobilized onto nitrocellulose membranes (GE Healthcare) by electroblot (Bio-Rad Laboratories). Specific proteins were detected by immunoblotting with the indicated antibodies (Table 4); immunoreactive proteins were detected by enhanced chemiluminescence (Thermo Scientific) on a ChemiDoc XRS+ (Bio-Rad Laboratories).

TABLE 4
Antibodies

| Antibody | Manufacturer | Protein | Species |
|---|---|---|---|
| SAB4300042 | Millipore-Sigma | PAKT (S142) | Rabbit |
| CST9167 | Cell Signaling | STAT1 | Mouse |
| | Technology | | |
| CST9167 | Cell Signaling | JAK1 | Mouse |
| | Technology | | |
| MA5-15739 | ThermoFisher | β-Actin | Mouse |
| | Scientific | | |
| CST14220 | Cell Signaling | Caspase-3 | Rabbit |
| | Technology | | |
| CST964 | Cell Signaling | Cleaved Caspase-3 | Rabbit |
| | Technology | | |
| CST942 | Cell Signaling | PARP | Rabbit |
| | Technology | | |
| CST5625 | Cell Signaling | Cleaved PARP | Rabbit |
| | Technology | | |

Growth Assays. Cell growth was measured either through live cell imaging or by cell counting at discrete points. Live cell imaging was used to assess the growth kinetics of SW-EV and SW-TRβ. To perform live cell imaging, 5000 cells were plated per well in 24 well plates. After 24 hours in phenol red free, charcoal stripped media, media was supplemented with either 10⁻⁸ M T₃ or equivolume vehicle (NaOH). Every two hours cells were imaged with a Lionheart automated microscope (BioTek) and quantified with Gen5 software (BioTek). The cells entered the log phase of growth at approximately 48 hours, and the portion of the curve following this point was used to calculate the doubling time of the cells. In order to measure growth manually, 25,000 cells were plated per well in a six well plate. Cells were grown in full thyroid media with the addition of 2-(1,8-Naphthyridin-2-yl)phenol (2-NP) (Abcam) or vehicle (DMSO). Every 24 hours cell counts were assessed by hemocytometer.

Soft Agar Colony Formation Assay. Soft agar colony forming assays were used to assess anchorage-independent growth. A layer 0.50% agar in thyroid media (as described) was solidified in 6-well cell culture plates. SW-EV or SW-TRβ cells were plated in a second layer of 0.25% agar in thyroid media. 200 uL of thyroid media was maintained at the top of each well to prevent the agar from drying. Where indicated, 10⁻⁸ M T₃ or vehicle (NaOH) was added to the media in all layers to evaluate the effects of liganded TRβ on anchorage-independent growth. Colonies were allowed to grow for 14 days. Live SW-EV and SW-TRβ colonies were detected via GFP expression using a ChemiDoc XRS+ (Bio-Rad Laboratories). Colonies were then counted with ImageJ using the Colony Counter plugin.

Tumorsphere Assay. Tumorsphere-forming assays were used to assess self-renewal and sphere-forming efficiency. For generating thyrospheres, adherent SW-EV and SW-TRβ monolayer cells were dissociated with Trypsin-EDTA, and single cells were moved to round-bottom ultra-low attachment 96-well plates at a density of 1000 cells/well (Corning, Corning, N.Y., USA). Thyrospheres were cultured in RPMI 1640 growth media with puromycin, supplemented with epidermal growth factor (EGF), and fibroblastic growth factor (FGF) (GoldBio) (20 ng/ml each). Where indicated, 10⁻⁸M T₃ or vehicle (NaOH) was added to the media to evaluate the effects of liganded TRβ on thyrosphere growth. Thyrospheres grew for seven days and were then counted with an inverted microscope at 10× magnification.

Migration Assay. Cell migration was determined by wound healing assay. Cells were plated and allowed to grow to 100% confluency. Two hours prior to scratching, cells were treated with 10 mg/ml Mitomycin C. A scratch was performed with a P1000 pipette tip and debris was washed away with PBS. Migration media was supplemented with 10⁻⁸ M T₃. Images were obtained at 0, 24, 48, and 72 hours. Wound closure was measured using ImageJ macro “Wound Healing Tool” (http://dev.mri.cnrs.fr/projects/imagej-macros/wiki/Wound_Healing_Tool). Values were normalized so that the initial scratch was 0% closure.

Apoptosis Assay. Cells were plated at a density of 50,000 cells/well and treated with vehicle (1 N NaOH) or 10⁻⁸ M T₃. After 5 days the cells were lysed for analysis by immunoblot. Poly (ADP-ribose) polymerase 1 (PARP1) and Caspase 3 cleavage were assessed to measure apoptotic signaling.

RNA Extraction and Quantitative Real-Time PCR (qRT-PCR). Total RNA was extracted using RNeasy Plus Kit (Qiagen) according to manufacturer's protocol. cDNA was then generated using the 5×RT Mastermix (ABM). Gene expression to validate RNASeq analysis was quantified by qRT-PCR using 2×SuperGreen Mastermix (ABM) on a QuantStudio 3 real-time PCR system (Applied Biosystems). Fold change in gene expression compared to endogenous controls was calculated using the ddCT method. Primer sequences are indicated in Table 5.

TABLE 5
Primers used for qRT-PCR Analysis

| Gene | Forward Oligo | Reverse Oligo |
|---|---|---|
| MYC | AGCGACTCTGAGGAGGGAACA | GTGGGCTGTGAGGAGGTTTG |
| | A (SEQ ID NO: 3) | (SEQ ID NO: 4) |
| CDKN1A | GCAGACCAGCATGACAGATTT | CTGGACTTCGAGCAAGAGATG |
| | (SEQ ID NO: 5) | (SEQ ID NO: 6) |
| TNFSF10 | CCGTCAGCTCGTTAGAAAGAT | TGTGTTGCTTCTTCCTCTGGT |
| | GAT (SEQ ID NO: 7) | (SEQ ID NO: 8) |
| APOL6 | CCTGTTCAAAGGCCACCCTA | TCAAATGATTTTCTTCTCTCC |
| | (SEQID NO: 9) | ACGG (SEQ ID NO: 10) |
| TAP1 | GCCTTGTTCCGAGAGCTGAT | AATGGCCATCTCCCCAAGAG |
| | (SEQID NO: 11) | (SEQ ID NO: 12) |
| IRF1 | CTTCCAGGTGTCACCCATGC | CCATCCACGTTTGTTGGCTG |
| | (SEQ ID NO: 13) | (SEQ ID NO: 14) |
| GAPDH | ATGTTCGTCATGGGTGTGAA | TGTGGTCATGAGTCCTTCCA |
| | (SEQ ID NO: 15) | (SEQID NO: 16) |

Statistics. All statistical analyses were performed using GraphPad Prism software. Paired comparisons were by T-test and group comparisons were made by a 2-way ANOVA followed by a Tukey multiple comparisons test (p<0.05). Data are represented as mean±standard deviation, or when stated otherwise mean±standard error of the mean. Area under the curve (AUC) at the 95^{th} confidence interval was used to evaluate statistical differences in growth and migration assays.

### Example 1—Expression of TRβ Inhibits Pro-Tumorigenic Characteristics of Human Anaplastic Thyroid Cells

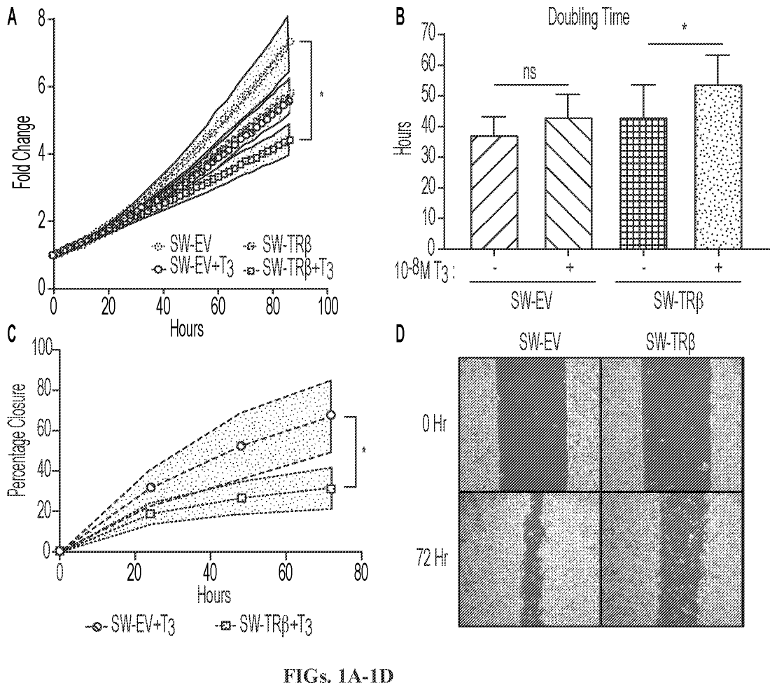

Anti-tumorigenic signaling from the tumor suppressor TRβ has not been comprehensively defined in ATC. The human ATC cell line SW1736, modified to overexpress TRβ (previously used in (Gillis et al., “Thyroid Hormone Receptor β Suppression of RUNX2 is Mediated by Brahma Related Gene 1 Dependent Chromatin Remodeling.” Endocrinol. 159(6):2484-94 (2018), which is hereby incorporated by reference in its entirety), was utilized in these studies to examine the effect of both T₃ and TRβ on the cellular growth. T₃ modestly decreased the growth rate of SW-EV (FIG. 1A). Vehicle treated SW-EV also grew faster than vehicle treated SW-TRβ. Importantly, the combination of TRβ overexpression and T₃ treatment profoundly reduced cellular growth. T₃ treated SW-TRβ also exhibited the greatest doubling time (FIG. 1B). TRβ expression and treatment with cognate ligand profoundly reduced the growth of these cells.

In addition to inducing anti-proliferative activity, TRβ is known to block metastasis in vivo (Martinez-Iglesias et al., “Hypothyroidism Enhances Tumor Invasiveness and Metastasis Development,” PLoS One 4(7):e6428 (2009), which is hereby incorporated by reference in its entirety). As metastasis and invasiveness require heightened cellular motility, the impact of T₃ and TRβ on ATC cell migration was also assessed by wound healing assay. Ligand treated SW-TRβ showed reduced migratory potential compared to ligand treated SW-EV (FIG. 1C-1D). These data indicate that TRβ in the presence of T₃ ligand exerts tumor suppressor activity in ATC cells.

### Example 2—TRβ Reprograms Anaplastic Thyroid Cancer Cell Signaling

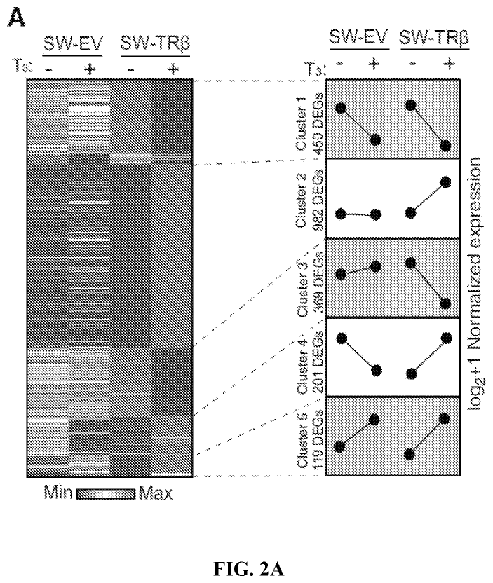

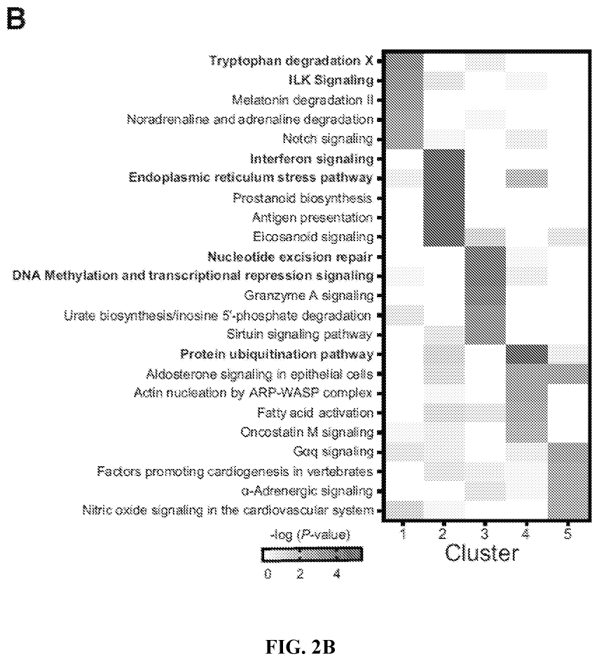

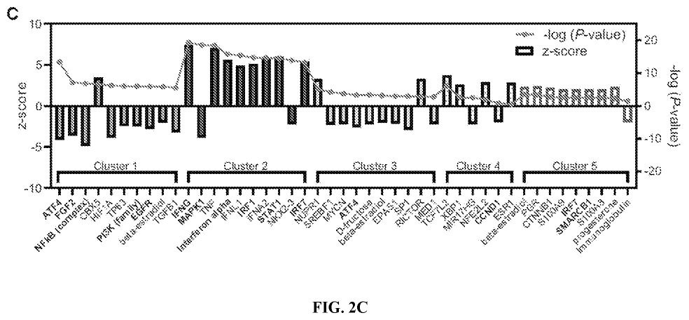

Given that T₃ significantly reduced ATC cell growth and migration, gene expression patterns elicited by TRβ with T₃ treatment were interrogated by RNAseq. The expression patterns of differentially expressed genes (DEGs) across each condition was examined via a clustered heatmap (FIG. 2A). This analysis resulted in 5 distinctive clusters of expression patterns. Clusters 1 and 5 are groups of genes that regulated by T₃ even in the absence of TRβ overexpression. Clusters 2 and 3 are genes that are T₃ regulated only when TRβ is overexpressed. Cluster 4 includes the genes that are repressed by T₃ in the absence of TRβ and induced by T₃ in cells that overexpress TRβ. These clusters of DEGs were used to determine IPA pathways (FIG. 2B). Notable processes include Integrin-linked kinase (ILK) signaling (cluster 1), interferon signaling (cluster 2), endoplasmic reticulum (ER) stress (cluster 2), nucleotide excision repair (NER) (cluster 3), DNA methylation and transcriptional repression (cluster 3), and protein ubiquitination (cluster 4). Of these pathways highlighted in the cluster analysis, TRβ has previously been shown to regulate ILK (hereafter PI3K/ILK) and interferon signaling (hereafter interferon/JAK1/STAT1) (Kim et al., “Inhibition of Tumorigenesis by the Thyroid Hormone Receptor Beta in Xenograft Models,” Thyroid Off. J. Amer. Thyroid Assoc. 24(2):260-9 (2014), Lopez-Mateo et al., “The Thyroid Hormone Receptor Beta Inhibits Self-Renewal Capacity of Breast Cancer Stem Cells,” Thyroid Off. J. Amer. Thyroid Assoc. (2019), which is hereby incorporated by reference in its entirety).

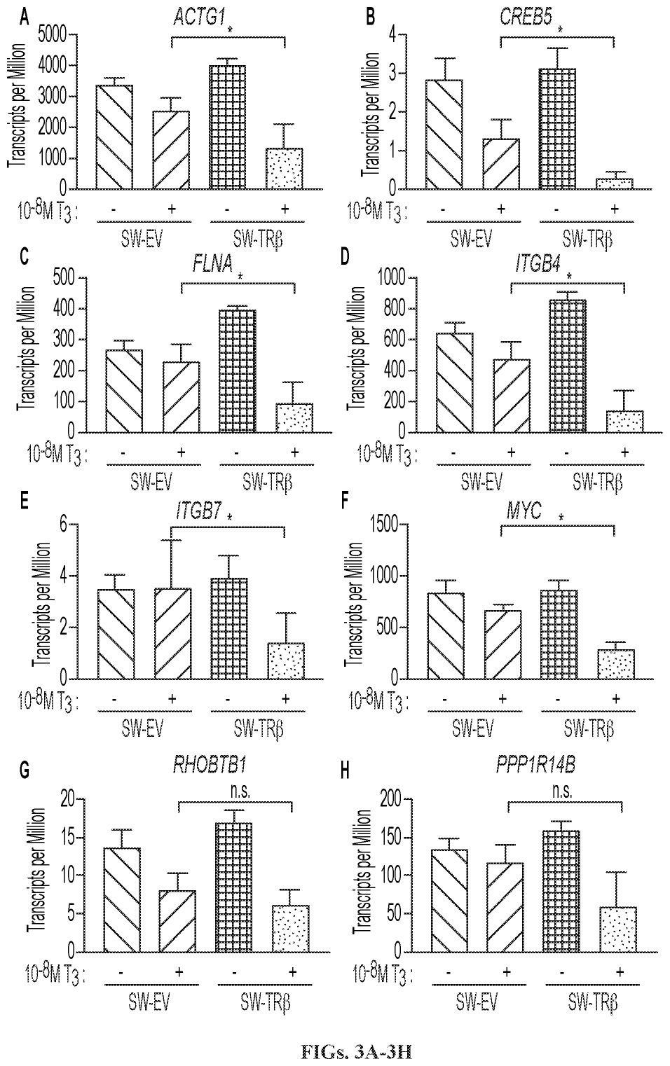

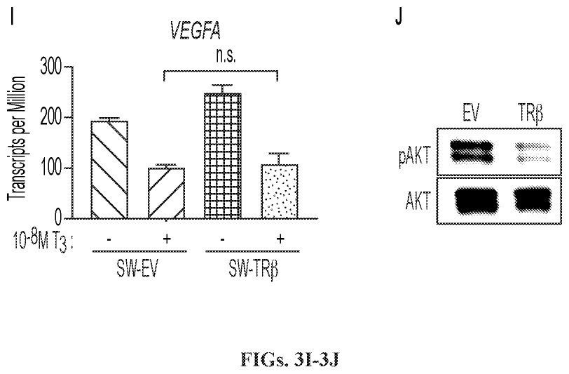

ILK is an enzyme that, following PIP2 phosphorylation by PI3K, acts to phosphorylate AKT, which then activates proliferative and invasive cellular processes (Yen et al., “Roles of Integrin-Linked Kinase in Cell Signaling and its Perspectives as a Therapeutic Target,” Gynecology Minimally Invasive Ther. 3(3):67-72 (2014), which is hereby incorporated by reference in its entirety). A pairwise comparison between T₃ treated SW-EV and SW-TRβ cells demonstrates that robust expression of TRβ further represses the PI3K/ILK gene cluster (FIG. 3A-3H). Therefore, changes to the PI3K/ILK pathway are likely a reflection of the known repressive effect TRβ has on PI3K in cancer (Kim et al., “Inhibition of Tumorigenesis by the Thyroid Hormone Receptor Beta in Xenograft Models,” Thyroid Off. J. Amer. Thyroid Assoc. 24(2):260-9 (2014), which is hereby incorporated by reference in its entirety). TRβ repression of PI3K signaling was confirmed by assessing pAKT/AKT (FIG. 3J).

TRβ has recently been shown to alter a set of genes in the interferon/JAK1/STAT1 pathway in breast cancer cells (Lopez-Mateo et al., “The Thyroid Hormone Receptor Beta Inhibits Self-Renewal Capacity of Breast Cancer Stem Cells,” Thyroid Off. J. Amer. Thyroid Assoc. (2019), which is hereby incorporated by reference in its entirety). This pathway was also a notable pathway altered by TRβ in the ATC cell line SW1736. Due to the transcriptomic profiling of SW1736 being performed in vitro, the interferon response here is most likely the intrinsic interferon pathway (Parker et al., “Antitumour Actions of Interferons: Implications for Cancer Therapy,” Nat. Rev. Cancer. 16(3):131-44 (2016), which is hereby incorporated by reference in its entirety). In the intrinsic interferon pathway, stimulation of the interferon receptor in cells drives phosphorylation of JAK proteins, which in turn phosphorylate STAT1 to initiate a transcriptional response. STAT1 signaling has been reported to promote apoptosis and differentiation of tumor cells, as well as inhibit growth (Parker et al., “Antitumour Actions of Interferons: Implications for Cancer Therapy,” Nat. Rev. Cancer. 16(3):131-44 (2016), which is hereby incorporated by reference in its entirety).

The upstream regulators of the clusters were determined using IPA (FIG. 2C). This analysis further confirmed alteration of the PI3K/ILK and interferon/JAK1/STAT1 pathways. Suppression of PI3K/ILK signaling was consistent with repression of the PI3K family and the receptor tyrosine kinases FGF2 and EGFR (cluster 1). There was predicted activation of multiple effectors within the interferon/JAK1/STAT1 signaling network, including IFNG, IRF1, IFNA2, and STAT1 in cluster 2 as well as IRF7 in clusters 2 and 5.

Analysis of the upstream regulators highlighted other genes known to be important in tumorigenesis. Notable upstream regulators were NF-κβ (cluster 1), MAPK1 (cluster 2), CCND1 (cluster 4), ATF4 (clusters 1 and 3), and SMARCB1 (cluster 5). NF-κβ and MAPK1 are well recognized to be oncogenic (Xing, “Molecular Pathogenesis and Mechanisms of Thyroid Cancer,” Nat. Rev. Cancer 13(3):184-99 (2013), Giuliani et al., “The Role of the Transcription Factor Nuclear Factor-kappa B in Thyroid Autoimmunity and Cancer,” Front. Endocrinol. 9:471 (2018), which is hereby incorporated by reference in its entirety) and were predicted to be repressed by TRβ and T₃. CCND1 encodes the protein cyclin D1, a cell cycle regulator which is regulated by T₃ and TRβ in other cell types (Pibiri et al., “Cyclin D1 is an Early Target in Hepatocyte Proliferation Induced by Thyroid Hormone (T3),” Faseb J. 15(6):1006-13 (2001), Porlan et al., “Thyroid Hormone Receptor-Beta (TR beta 1) Impairs Cell Proliferation by the Transcriptional Inhibition of Cyclins D1, E and A2,” Oncogene 27(19):2795-800 (2008), which is hereby incorporated by reference in its entirety), and was predicted to be repressed in this analysis. The ER stress regulator ATF4 was repressed, itself a gene that is typically upregulated in malignancy and a potential drug target for ATC (Wortel et al., “Surviving Stress: Modulation of ATF4-Mediated Stress Responses in Normal and Malignant Cells,” Trends Endocrinol. Metab. 28(11):794-806 (2017), Mehta et al., “Carfilzomib is an Effective Anticancer Agent in Anaplastic Thyroid Cancer,” Endocr. Relat. Cancer 22(3):319-29 (2015), which is hereby incorporated by reference in its entirety). Genes positively regulated by SMARCB1, a component of the SWI/SNF chromatin remodeling complex, were induced by T₃. Components of SWI/SNF are commonly mutated in ATC compared to other thyroid cancer subtypes (Landa et al., “Genomic and Transcriptomic Hallmarks of Poorly Differentiated and Anaplastic Thyroid Cancers,” J. Clin. Invest. 126(3):1052-66 (2016), which is hereby incorporated by reference in its entirety). A pairwise comparison between SW-EV-T₃ and SW-TRβ-T₃ was performed in order to further understand the role of TRβ activity. The DEGs were examined via IPA and GSEA using both the Hallmarks and Gene Ontology datasets.

This analysis further confirmed the interferon signaling network was induced, as observed from the analysis of cluster 2. Additionally, in the Gene Ontology analysis were multiple pathways relating to autophagic signaling, a process known to be altered by T₃ in other cell lines (Chi et al., “Thyroid Hormone Suppresses Hepatocarcinogenesis via DAPK2 and SQSTM1-Dependent Selective Autophagy,” Autophagy (12):2271-85 (2016), which is hereby incorporated by reference in its entirety) and a process dysregulated in tumors (Levy et al., “Targeting Autophagy in Cancer,” Nat. Rev. Cancer 17(9):528-42 (2017), which is hereby incorporated by reference in its entirety) and a process dysregulated in tumors (Levy et al., “Targeting Autophagy in Cancer,” Nat. Rev. Cancer 17(9):528-42 (2017), which is hereby incorporated by reference in its entirety). These results are suggestive of TRβ and T₃ repressing pro-tumorigenic activity in the cells.

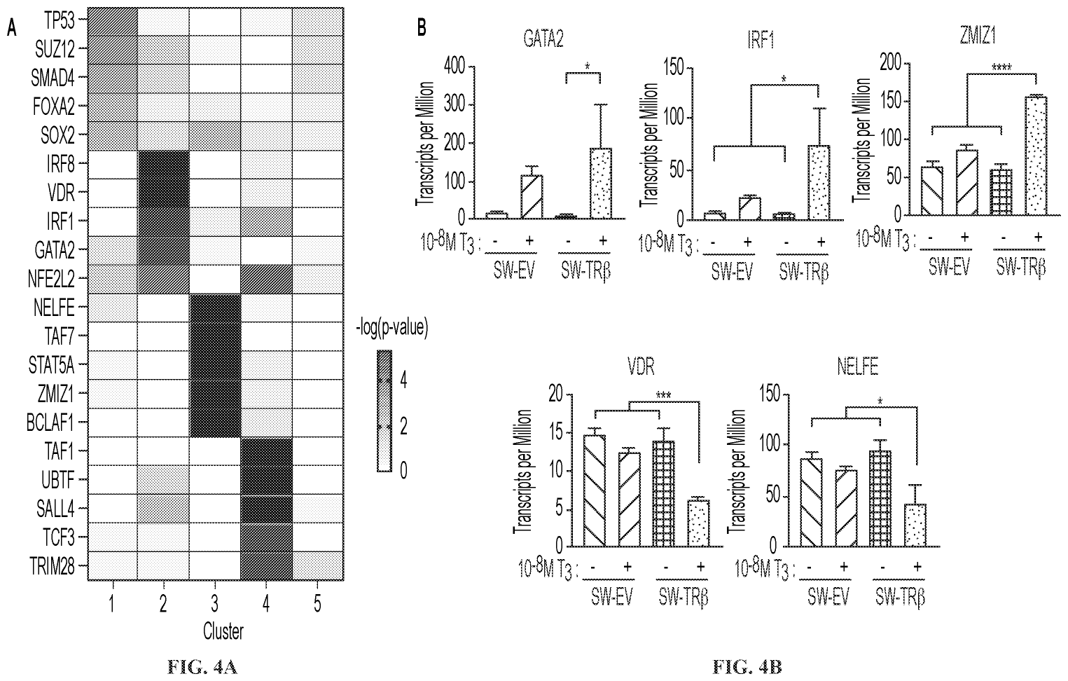

The gene clusters were also used for chromatin immunoprecipitation enrichment analysis (ChEA) (Lachmann et al., “Transcription Factor Regulation Inferred from Integrating Genome-Wide Chip-X Experiments,” Bioinformatics 26(19):2438-444 (2010), which is hereby incorporated by reference in its entirety) in order to determine which transcription factors have overrepresented binding sites (FIGS. 4A-4B). Of these transcription factors, GATA2, IRF1, NELFE, VDR, and ZMIZ1 exhibited altered expression and are therefore possible drivers of TRβ-mediated signaling.

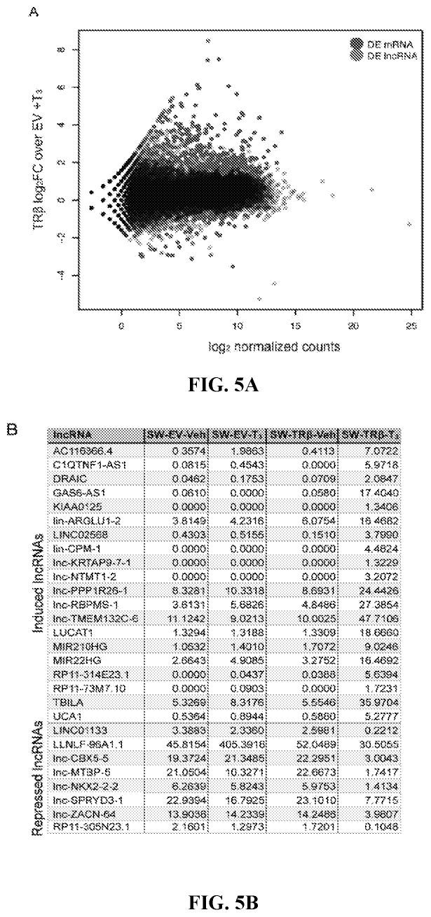

Due to the majority of the DEGs bellowing to clusters that were only significantly altered in the TRβ-T₃ condition, a pairwise comparison between SW-EV-T₃ and SW-TRβ-T₃ was performed (FIGS. 5A-5B). The long noncoding RNAs (lncRNA) were pulled from the set of genes due to their emerging role in tumor biology (Sanchez et al., “Emerging Roles of Long Non-Coding RNA in Cancer,” Cancer Sci. 109(7):2093-100 (2018), which is hereby incorporated by reference in its entirety). lncRNAs upregulated included C1QTNF1-AS1, MIR210HG, TBILA, GAS6-AS1, LUCAT1, DRAIC, KIAA0125, MIR22HG, and UCA1. Conversely, LINC01133 was repressed. These lncRNAs have all been found to have a role in tumorigenesis (Li H et al., “C1QTNF1-AS1 Regulates the Occurrence and Development of Hepatocellular Carcinoma by Regulating miR-221-3p/SOCS3,” Hepatol Int. 13(3):277-92 (2019), Li et al., “Long Noncoding RNA miR210HG Sponges miR-503 to Facilitate Osteosarcoma Cell Invasion and Metastasis,” DNA Cell Biol. 36(12):1117-25 (2017), Lu et al., “The TGFbeta-induced lnc RNA TBILA Promotes Non-Small Cell Lung Cancer Progression in vitro and in vivo via Cis-Regulating HGAL and Activating S100A7/JAB1 Signaling,” Cancer Lett. 432:156-68 (2018), Sun et al., “Long Non-Coding RNA LUCAT1 is Associated with Poor Prognosis in Human Non-Small Lung Cancer and Regulates Cell Proliferation via Epigenetically Repressing p21 and p57 Expression,” Oncotarget. 8(17):28297-311 (2017), Zhao et al., “Upregulation of Long Non-Coding RNA DRAIC Correlates with Adverse Features of Breast Cancer,” Noncoding RNA 4(4) (2018), Yang et al., “lncRNA KIAA0125 Functions as a Tumor Suppressor Modulating Growth and Metastasis of Colorectal Cancer via Wnt/beta-catenin Pathway,” Cell Biol Int. (2019), Zhang et al., “Identification and Functional Characterization of Long Non-Coding RNA MIR22HG as a Tumor Suppressor for Hepatocellular Carcinoma,” Theranostics 8(14):3751-5 (2018), Wang et al., “LncRNA UCA1 in Anti-Cancer Drug Resistance,” Oncotarget 8(38):64638-50 (2017), Yang et al. “LINC01133 as ceRNA Inhibits Gastric Cancer Progression by Sponging miR-106a-3p to Regulate APC Expression and the Wnt/beta-catenin Pathway,” Mol Cancer. 17(1):126 (2018), which is hereby incorporated by reference in its entirety).

Analysis of the transcriptomic profile induced by overexpression of TRβ and T₃ shows that many pathways and key regulators in cancer biology are altered. These signaling nodes relate to survival signaling, invasiveness, cellular maintenance, differentiation, and chromatin organization. Thus, T₃ treatment of SW-TRβ induces transcriptomic changes associated with reduced cell growth, migration, cell cycle, and cell survival.

### Example 3—Liganded TRβ Reduces ATC Stem-Cell Characteristics

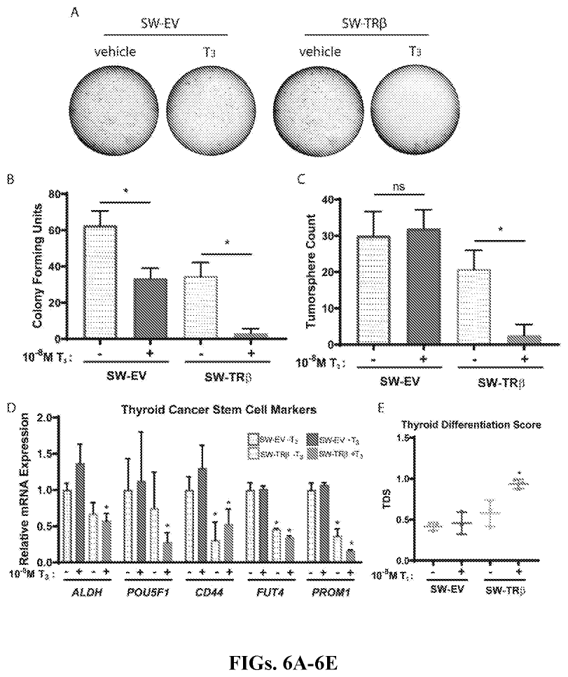

Cancer stem cells are thought to be more prevalent in ATC than in other subtypes of thyroid cancer due to its aggressive and mesenchymal phenotype (Todaro et al., “Tumorigenic and Metastatic Activity of Human Thyroid Cancer Stem Cells,” Cancer Res. 70(21):8874-85(2010), which is hereby incorporated by reference in its entirety). Recently, it was demonstrated that TRβ to reduces cancer stem cell renewal in luminal breast cancer cell lines (Lopez-Mateo et al., “The Thyroid Hormone Receptor Beta Inhibits Self-Renewal Capacity of Breast Cancer Stem Cells,” Thyroid Off. J. Amer. Thyroid Assoc. (2019), which is hereby incorporated by reference in its entirety). Whether TRβ expression and T₃ treatment could attenuate the stem-like properties SW1736 cells was examined. Soft agar colony formation assays were used to measure changes in anchorage independent growth in the presence and absence of TRβ and T₃. Treatment with T₃ reduced anchorage-independent colony growth in SW-EV cells, and nearly ablated colony growth in SW-TRβ cells (FIG. 6A-6B). Tumorsphere assays were used to assess self-renewal and estimate the size of the cancer stem cell population within the heterogeneous cultures. T₃ had no effect on the number of tumorspheres formed from SW-EV cells. Expression of TRβ alone reduced tumorsphere formation, however the addition of T₃ blocked almost all tumorsphere growth (FIG. 6C).

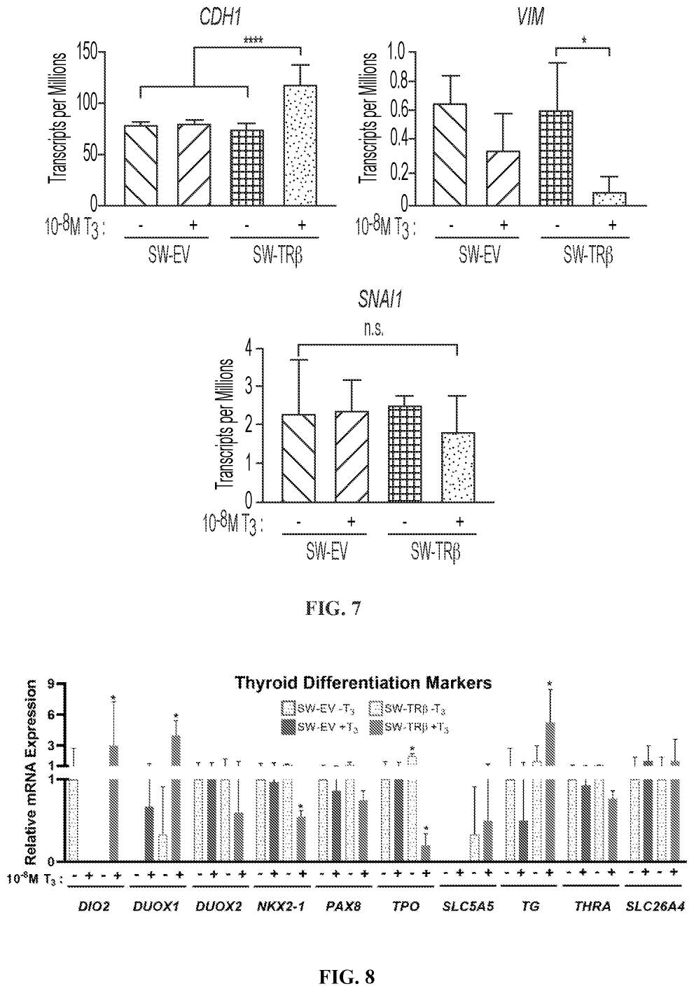

Expression of key stem cell markers was also altered. Although the markers for thyroid cancer stem cells have not been characterized with the same rigor as other aggressive cancer types, a recent review compiled a list of proposed cancer stem cell markers that have been characterized in the thyroid (Nagayama et al., “Cancer Stem Cells in the Thyroid,” Front Endocrinol (Lausanne) 7:20 (2016), which is hereby incorporated by reference in its entirety). The combination of TRβ overexpression and T₃ significantly reduced expression of the thyroid-specific cancer stem cell genes ALDH, POU5F1 (encodes OCT3/4), CD44, FUT4 (encodes SSEA-1), and PROM1 (encodes CD133) (FIG. 6D). The EMT markers CHD1 (E-cadherin), VIM (Vimentin) were also examined (FIG. 7). The epithelial marker CDH1 exhibited increased expression in SW1736-TRβ T₃ treated cells, whereas expression of the mesenchymal marker VIM was decreased. Together with the phenotypic assays, these results demonstrate that TRβ in association with ligand decreases the stem cell population of ATC cells.

To further explore the impact of T₃ treatment in this ATC model, the thyroid differentiation score (TDS) was calculated for the SW1736 cells under each condition to evaluate whether the transcriptomic data indicates a change in thyroid-specific differentiation. The TDS has been demonstrated to have utility in predicting the aggressiveness of a thyroid tumor (Cancer Genome Atlas Research Network, “Integrated Genomic Characterization of Papillary Thyroid Carcinoma,” Cell 159(3):676-90 (2014), Landa et al., “Comprehensive Genetic Characterization of Human Thyroid Cancer Cell Lines: A Validated Panel for Preclinical Studies,” Clin. Cancer Res. Offic. J. Amer. Assoc. Cancer Res. 25(10):3141-51 (2019), which is hereby incorporated by reference in its entirety). Ten out of the thirteen genes which constitute the TDS are expressed in at least one condition in this dataset. DIO2, DUOX1, TPO, and TG, were highly responsive to TRβ expression and the addition of T₃ (FIG. 8). SW-TRβ cells treated with T₃ have a significantly higher TDS, indicating that they have the most thyroid-like gene expression (FIG. 6E). These results suggest that the tumor suppressive activity of TRβ observed in SW1736 cells may involve cellular differentiation and a consequential reduction in the cancer stem cell properties.

### Example 4—TRβ Stimulates Apoptotic Signaling

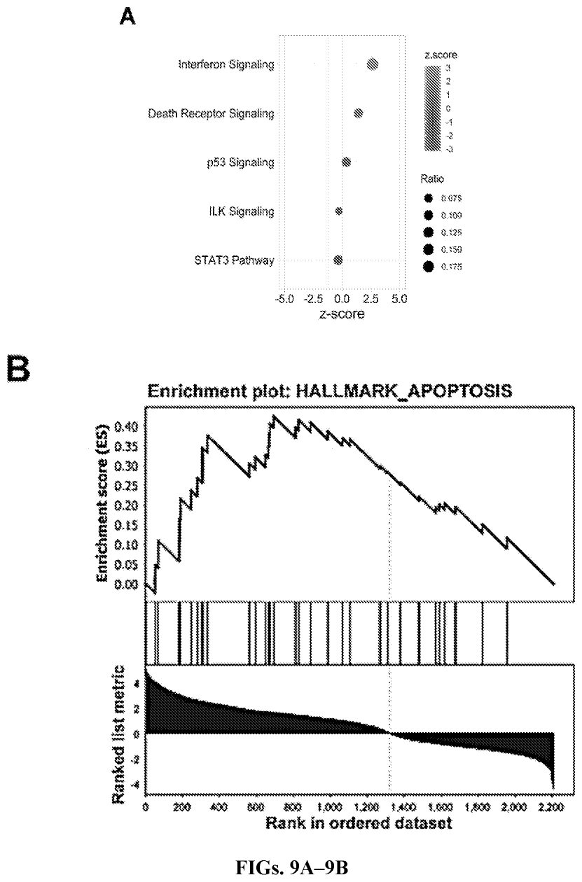

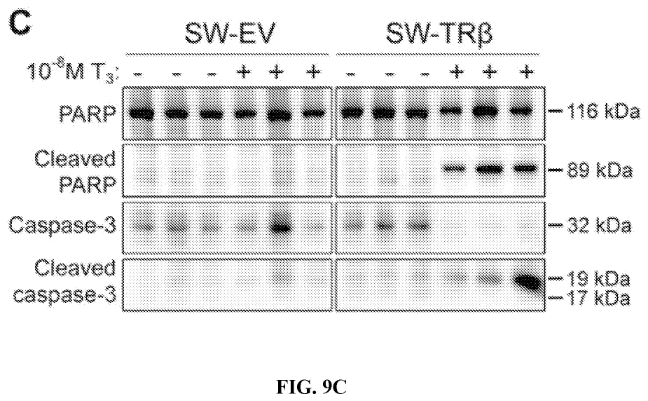

As a tumor suppressor, TRβ potentiates cancer cells for apoptosis, although it is not clear if this process occurs in ATC (Zhu et al., “Synergistic Signaling of KRAS and Thyroid Hormone Receptor Beta Mutants Promotes Undifferentiated Thyroid Cancer Through MYC up-Regulation,” Neoplasia 16(9):757-69 (2014), Park et al., “Oncogenic Mutations of Thyroid Hormone receptor β,” Oncotarget 6(10):8115-31 (2015), which is hereby incorporated by reference in its entirety). Multiple pathways involved in apoptotic signaling were revealed from a direct comparison of the DEGs from SW-EV and SW-TRβ cells treated with T₃ (FIG. 9A). Additionally, GSEA analysis using Hallmark gene sets resulted in enrichment of apoptotic signaling (FIG. 9B). The ability of T₃ to stimulate apoptotic signaling pathways in SW-TRβ was further investigated. SW-EV and SW-TRβ cells were cultured in media supplemented with T₃ and caspase 3 and PARP1 cleavage were measured to assess apoptotic activity. At 5 days, T₃ induced cleavage of these apoptotic effectors only in SW-TRβ (FIG. 9C). Neither T₃ nor TRβ alone was sufficient to elicit this response. These data demonstrate heightened activity of TRβ promotes an apoptotic response in aggressive cancer cells, further confirming tumor suppressive activity.

### Example 5—TRβ Promotes Anti-Proliferative Signaling

Of the pathways altered by TRβ in SW1736, the interferon/JAK1/STAT1 pathway was particularly prominent. Interferon/JAK1/STAT1 signaling has potent anti-tumor and anti-proliferative activity (Parker et al., “Antitumour Actions of Interferons: Implications for Cancer Therapy,” Nat. Rev. Cancer. 16(3):131-44 (2016), Meissl et al., “The Good and the Bad Faces of STAT1 in Solid Tumours,” Cytokine 89:12-20 (2017), which is hereby incorporated by reference in its entirety). Interferon alpha and gamma pathways were also predicted from the Hallmark gene set utilizing GSEA to be upregulated through a pairwise comparison of ligand treated SW-EV and SW-TRβ (see Table 6 herein) The primary effector of the interferon response is the transcription factor STAT1. Thus it was decided to investigate the activity of this pathway in ATC and whether it could be modulated to alter cellular characteristics.

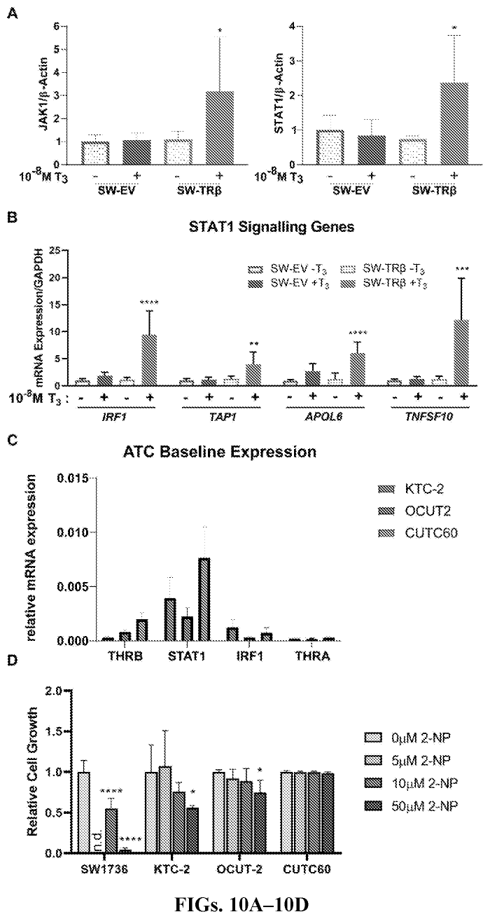

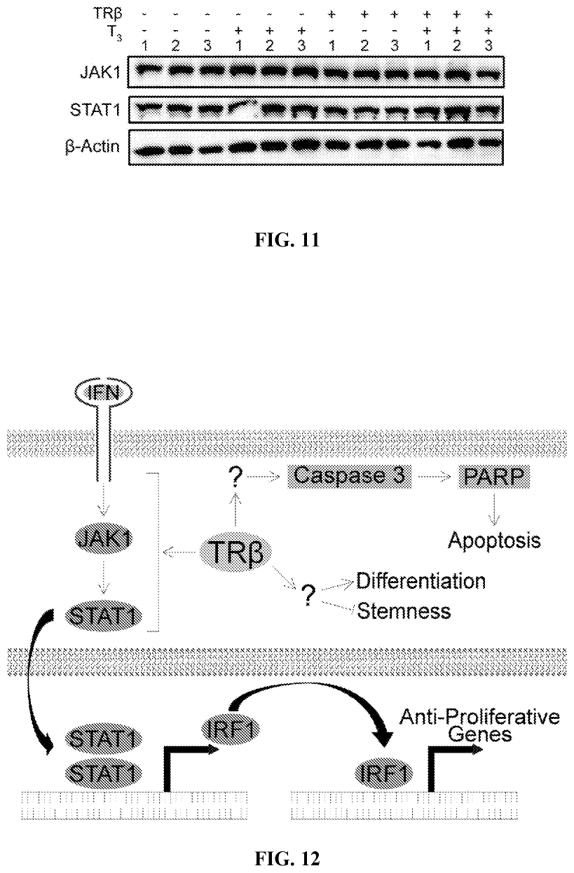

Expression of the kinase effectors JAK1 and STAT1 were significantly increased in SW-TRβ cells following treatment with T₃ (FIG. 10A and FIG. 11). Next, transcriptional targets of the interferon/JAK1/STAT1 pathway were assessed by qPCR to determine the role of T₃ signaling. T₃ increased the expression of STAT1 target genes IRF1 and TAP1 in SW-TRβ cells. Induction of IRF1 target genes APOL6 and TNFSF10 was also demonstrated (FIG. 10B).

STAT1 has anti-proliferative activity, raising the question whether it can be exploited pharmacologically to reduce ATC growth. Baseline expression of key genes in the JAK1-STAT1 pathway and TR isoform levels (TRβ and TRα) in 3 additional anaplastic thyroid cancer cell lines are illustrated in FIG. 10C. To identify the effects of STAT1 stimulation on ATC cells, the STAT1 activator 2-(1,8-naphthyridin-2-yl)phenol (2-NP) ((Lynch et al., “A Small-Molecule Enhancer of Signal Transducer and Activator of Transcription 1 Transcriptional Activity Accentuates the Antiproliferative Effects of IFN-gamma in Human Cancer Cells,” Cancer Res. 67(3):1254-61 (2007), which is hereby incorporated by reference in its entirety) was administered to SW1736, KTC-2, OCUT-2 and CUT60 cells (0 μM, 5 μM, 10 μM, or 50 μM) for 24 hours. As shown in FIG. 10D, activation of STAT1 recapitulates the effect of T₃-TRβ in SW1736, KTC-2, OCUT-2 but not CUTC60 indicating that induction of apoptotic signaling through enhanced JAK1-STAT1 activity is independent of TP53 mutational status

TABLE 6
Summary of Ingenuity Pathway Analysis

| Ingenuity Canonical Pathways | -log(p-value) | Ratio | z-score | Molecules |
|---|---|---|---|---|
| LPS/IL-1 Mediated Inhibition of RXR Function | 7.75E+00 | 1.25E−01 | −1.265 | ABCB1, ABCC2, ABCC4, ACSL6, ALDH1A1, ALDH1A3, |
| | | | | ALDH1L1, ALDH1L2, ALDH3A1, ALDH3B2, CD14, CHST11, |
| | | | | CHST7, CPT1C, CYP2B6, CYP3A7, FABP5, FMO5, GSTA1, |
| | | | | IL1A, MAOA, PPARGC1A, PPARGC1B, SREBF1, |
| | | | | SULT1A1, SULT1A2, SULT1C2, TNFRSF11B |
| VDR/RXR Activation | 3.88E+00 | 1.41E−01 | 0.378 | CD14, CDKN1A, CEBPA, CST6, CYP24A1, IL12A, KLK6, |
| | | | | PPARD, RUNX2, SPP1, VDR |
| Prostanoid Biosynthesis | 3.70E+00 | 4.44E−01 | 2 | PTGDS, PTGES, PTGS1, TBXAS1 |
| Interferon Signaling | 3.53E+00 | 1.94E−01 | 2.646 | IFI6, IFIT1, IFIT3, IFITM3, IRF1, PSMB8, TAP1 |
| Dopamine Degradation | 3.16E+00 | 2.00E−01 | 0 | ALDH1A1, ALDH1A3, ALDH3A1, MAOA, SULT1A1, |
| | | | | SULT1A2 |
| Aryl Hydrocarbon Receptor Signaling | 3.08E+00 | 9.79E−02 | −0.816 | ALDH1A1, ALDH1A3, ALDH1L1, ALDH1L2, ALDH3A1, |
| | | | | ALDH3B2, CDKN1A, CTSD, CYP1B1, GSTA1, IL1A, MYC, |
| | | | | NR2F1, TGFB3 |
| Cardiac Hypertrophy Signaling (Enhanced) | 2.91E+00 | 6.57E−02 | 0.365 | ADRA2A, ADRA2C, CAMK2B, CXCL8, FGF19, FGFR3, |
| | | | | FGFR4, FGFRL1, FZD7, GNAI1, IL12A, IL15RA, IL17RB, |
| | | | | IL1A, IL20RA, IL6R, MAP3K12, MAP3K5, MAPK10, MPPE1, |
| | | | | MYC, PDE4D, PDE5A, PIK3CD, PLCE1, TGFB3, TNFRSF11B, |
| | | | | TNFSF10, TNFSF15, WNT10A, WNT11, WNT4 |
| STAT3 Pathway | 2.83E+00 | 9.63E−02 | −0.333 | BMPR1B, CDKN1A, FGFR3, FGFR4, IL15RA, IL17RB, |
| | | | | IL1A, IL20RA, IL6R, MAP3K12, MAPK10, MYC, PIM1 |
| SPINK1 Pancreatic Cancer Pathway | 2.81E+00 | 1.33E−01 | −2.828 | CELA3A, CELA3B, CPM, KLK10, KLK11, KLK6, KLK7, KLK8 |
| Tryptophan Degradation X (Mammalian, via | 2.71E+00 | 2.00E−01 | −1.342 | ALDH1A1, ALDH1A3, ALDH3A1, DDC, MAOA |
| Tryptamine) | | | | |
| Inhibition of Matrix Metalloproteases | 2.55E+00 | 1.54E−01 | −1.342 | HSPG2, MMP13, MMP24, MMP25, MMP7, TIMP2 |
| Sirtuin Signaling Pathway | 2.54E+00 | 7.19E−02 | −0.229 | ACSS2, ATG4A, ATG9B, CPT1C, CXCL8, GABARAPL1, |
| | | | | GADD45B, GLS, HIF1A, HIST1H1B, HIST1H1C, HIST1H1D, |
| | | | | MAPK6, MYC, NDRG1, NDUFAF2, PCK2, PFKFB3, |
| | | | | PPARGC1A, SCNN1A, SREBF1 |
| Intrinsic Prothrombin Activation Pathway | 2.38E+00 | 1.43E−01 | 1.633 | COL18A1, KLK10, KLK11, KLK6, KLK7, KLK8 |
| Oxidative Ethanol Degradation III | 2.34E+00 | 2.11E−01 | 0 | ACSS2, ALDH1A1, ALDH1A3, ALDH3A1 |
| Osteoarthritis Pathway | 2.24E+00 | 7.51E−02 | 0.775 | ACVRL1, CREB5, CXCL8, FGFR3, FN1, FZD7, GLI1, HIF1A, |
| | | | | IHH, JAG1, MMP13, NOTCH1, PPARD, PPARGC1A, |
| | | | | RUNX2, SPP1 |
| Colorectal Cancer Metastasis Signaling | 2.19E+00 | 7.09E−02 | 2.5 | FZD7, GRK3, IL6R, MAPK10, MMP13, MMP24, MMP25, |
| | | | | MMP7, MYC, PIK3CD, RHOF, RHOU, RND3, TGFB3, |
| | | | | TLR3, WNT10A, WNT11, WNT4 |
| Putrescine Degradation III | 2.18E+00 | 1.90E−01 | −1 | ALDH1A1, ALDH1A3, ALDH3A1, MAOA |
| Dermatan Sulfate Biosynthesis (Late Stages) | 2.18E+00 | 1.30E−01 | 0 | CHST11, CHST7, DSEL, SULT1A1, SULT1A2, SULT1C2 |
| Ethanol Degradation IV | 2.03E+00 | 1.74E−01 | 0 | ACSS2, ALDH1A1, ALDH1A3, ALDH3A1 |
| p53 Signaling | 1.99E+00 | 9.18E−02 | 0.378 | CDKN1A, DRAM1, GADD45B, HIF1A, HIPK2, PIK3CD, |
| | | | | SERPINE2, TP53I3, TP53INP1 |
| Neuroprotective Role of THOP1 in Alzheimer's | 1.97E+00 | 8.62E−02 | 3 | DPP4, HLA-G, KLK10, |
| Disease | | | | KLK11, KLK6, KLK7, KLK8, PRSS22, PRSS23, |
| | | | | TMPRSS2 |
| Wnt/Î2-catenin Signaling | 1.92E+00 | 7.51E−02 | 0 | FZD7, KREMEN1, MMP7, MYC, PPARD, PPP2R5B, SOX18, |
| | | | | SOX8, TGFB3, TLE3, WNT10A, WNT11, WNT4 |
| Sumoylation Pathway | 1.86E+00 | 8.74E−02 | 0.333 | CEBPA, MAP3K5, MAPK10, MYB, RHOF, RHOU, RND3, |
| | | | | SLC19A1, ZEB1 |
| LXR/RXR Activation | 1.85E+00 | 8.26E−02 | 0.707 | APOL1, CD14, HPX, IL1A, PCYOX1, SCD, SERPINF2, |
| | | | | SREBF1, TLR3, TNFRSF11B |
| Leukocyte Extravasation Signaling | 1.80E+00 | 7.04E−02 | 2.111 | CLDN12, CLDN9, GNAI1, MAPK10, MMP13, MMP24, |
| | | | | MMP25, MMP7, NOX1, PIK3CD, RASSF5, RDX, TIMP2, |
| | | | | TXK |
| Death Receptor Signaling | 1.72E+00 | 8.79E−02 | 1.414 | BIRC3, MAP3K5, MAP4K4, PARP10, PARP14, PARP16, |
| | | | | TNFSF10, TNFSF15 |
| Endocannabinoid Neuronal Synapse Pathway | 1.69E+00 | 7.81E−02 | 1.667 | CACNA1H, CACNG6, CACNG8, DAGLA, GNAI1, GNAL, |
| | | | | MAPK10, MAPK6, MGLL, PLCE1 |
| HMGB1 Signaling | 1.69E+00 | 7.23E−02 | 0.632 | CXCL8, IL12A, IL1A, MAPK10, PIK3CD, RHOF, RHOU, |
| | | | | RND3, TGFB3, TNFRSF11B, TNFSF10, TNFSF15 |
| Dermatan Sulfate Biosynthesis | 1.67E+00 | 1.02E−01 | 0 | CHST11, CHST7, DSEL, SULT1A1, SULT1A2, SULT1C2 |
| Production of Nitric Oxide and Reactive Oxygen | 1.63E+00 | 6.88E−02 | 0.832 | APOL1, IRF1, MAP3K12, MAP3K5, MAPK10, PCYOX1, |
| Species in Macrophages | | | | PIK3CD, PPP1R14D, PPP2R5B, RHOF, RHOU, RND3, |
| | | | | TNFRSF11B |
| PCP pathway | 1.61E+00 | 9.84E−02 | 0.816 | FZD7, MAPK10, PRICKLE1, WNT10A, WNT11, WNT4 |
| ILK Signaling | 1.59E+00 | 6.77E−02 | −0.277 | CREB5, FN1, HIF1A, ITGB4, ITGB6, MAPK10, MUC1, |
| | | | | MYC, PIK3CD, PPP2R5B, RHOF, RHOU, RND3 |
| Ethanol Degradation II | 1.54E+00 | 1.25E−01 | 0 | ACSS2, ALDH1A1, ALDH1A3, ALDH3A1 |
| Apelin Adipocyte Signaling Pathway | 1.53E+00 | 8.64E−02 | 1.134 | GNAI1, GSTA1, HIF1A, MAPK10, MAPK6, NOX1, PPAR |
| | | | | GC1A |
| Chondroitin Sulfate Biosynthesis (Late Stages) | 1.51E+00 | 1.04E−01 | 0.447 | CHST11, CHST7, SULT1A1, SULT1A2, SULT1C2 |
| Superpathway of Melatonin Degradation | 1.49E+00 | 9.23E−02 | 0 | CYP1B1, CYP2B6, CYP3A7, MAOA, SULT1A1, SULT1A2 |
| IL-8 Signaling | 1.46E+00 | 6.50E−02 | 1.941 | ANGPT2, CXCL8, GNAI1, HBEGF, HMOX1, MAP4K4, |
| | | | | MAPK10, NOX1, PIK3CD, PLD4, RHOF, RHOU, RND3 |
| FGF Signaling | 1.43E+00 | 8.24E−02 | −1.134 | CREB5, FGF19, FGFR3, FGFR4, FGFRL1, MAP3K5, |
| | | | | PIK3CD |
| Serotonin Degradation | 1.43E+00 | 8.96E−02 | 0 | ALDH1A1, ALDH1A3, ALDH3A1, MAOA, SULT1A1, |
| | | | | SULT1A2 |
| Noradrenaline and Adrenaline Degradation | 1.41E+00 | 1.14E−01 | −1 | ALDH1A1, ALDH1A3, ALDH3A1, MAOA |
| Type II Diabetes Mellitus Signaling | 1.41E+00 | 6.99E−02 | −0.447 | ACSL6, CACNA1H, CACNG6, CACNG8, MAP3K5, |
| | | | | MAPK10, PIK3CD, SLC2A2, SMPD1, TNFRSF11B |
| B Cell Receptor Signaling | 1.36E+00 | 6.45E−02 | 0.905 | CAMK2B, CARD10, CD22, CREB5, IGHE, INPP5J, |
| | | | | MAP3K12, MAP3K5, PAG1, PIK3AP1, PIK3CD, RASSF5 |
| IL-6 Signaling | 1.36E+00 | 7.14E−02 | 0.707 | ABCB1, CD14, CXCL8, IL1A, IL6R, MAP4K4, MAPK10, |
| | | | | PIK3CD, TNFRSF11B |
| Glioblastoma Multiforme Signaling | 1.36E+00 | 6.63E−02 | 0.302 | CDKN1A, FZD7, MYC, PIK3CD, PLCE1, RHOF, RHOU, |
| | | | | RND3, WNT10A, WNT11, WNT4 |
| Opioid Signaling Pathway | 1.35E+00 | 6.00E−02 | 0.258 | CACNA1H, CACNG6, CACNG8, CAMK2B, CLTCL1, |
| | | | | CREB5, GNAI1, GNAL, GRK3, MAPK6, MYC, NPBWR1, |
| | | | | OPRL1, RGS11, RGS16 |

### Discussion of Examples 1-5:

While TRβ is a recognized tumor suppressor in thyroid cancer and is known to be silenced in ATC (Carr et al., “Thyroid Hormone Receptor-beta (TRbeta) Mediates Runt-Related Transcription Factor 2 (Runx2) Expression in Thyroid Cancer Cells: A Novel Signaling Pathway in Thyroid Cancer.” Endocrinol. 157(8):3278-92 (2016), which is hereby incorporated by reference in its entirety), TRβ-mediated signaling and phenotypic effects are not well characterized in ATC. The data herein in ATC cells demonstrates that TRβ, in conjunction with T₃, acts to regulate pathways important for the process of tumorigenesis and reduces the aggressive phenotypic characteristics. This change was demonstrated via multiple measures including reduced growth, migration, and stemness. In addition, the combination of TRβ and T₃ induced the expression of thyroid differentiation markers in ATC cells, and after 5 days resulted in apoptosis. Indeed, T₃ was essential for the profound anti-tumorigenic molecular signaling and phenotypic remodeling observed in this study. The major findings of this analysis are represented in FIG. 12.

The transcriptomic results reveal new details about TRβ-mediated regulation of critical cancer-related pathways in ATC. The prominent pathways from this analysis were repression of PI3K and activation of STAT1. TRβ-mediated repression of PI3K has been demonstrated in breast cancer and differentiated thyroid tumors (Kim et al., “Inhibition of Tumorigenesis by the Thyroid Hormone Receptor Beta in Xenograft Models,” Thyroid Off. J. Amer. Thyroid Assoc. 24(2):260-9 (2014), Park et al., “Oncogenic Mutations of Thyroid Hormone receptor β,” Oncotarget 6(10):8115-31 (2015), which is hereby incorporated by reference in its entirety), suggestive that this signaling is active in a variety of tumor types. Additionally, the data herein highlight the interferon/JAK1/STAT1 pathway, previously unknown to be modulated by T₃ in thyroid cells, although STAT1 was recently shown to be activated upon overexpression of TRβ in estrogen receptor positive breast cancer (Lopez-Mateo et al., “The Thyroid Hormone Receptor Beta Inhibits Self-Renewal Capacity of Breast Cancer Stem Cells,” Thyroid Off. J. Amer. Thyroid Assoc. (2019), which is hereby incorporated by reference in its entirety). Evidence of MAPK repression was also observed, another pathway previously shown to be regulated by TRβ in breast cancer cells (Garcia-Silva et al., “The Thyroid Hormone Receptor is a Suppressor of Ras-Mediated Transcription, Proliferation, and Transformation,” Mol. Cell. Biol. 24(17):7514-23 (2004), which is hereby incorporated by reference in its entirety). Along with alterations to PI3K and interferon activity, there were changes to cellular repair processes driven by TRβ overexpression. TRβ and T₃ altered expression of genes involved ER stress. This is consistent with alteration of protein ubiquitination, which may indicate a greater turnover rate of misfolded proteins, an important component of ER stress. Additionally, alteration of transcriptional repression and NER are consistent with observed reduction of cell growth. These pathways can initiate an apoptotic response, such as through an accumulation of misfolded proteins in the ER (Yadav et al., “Endoplasmic Reticulum Stress and Cancer,” J Cancer Prev. 19(2):75-88 (2014), which is hereby incorporated by reference in its entirety), or a failure of DNA repair pathways to repair damaged DNA (Nowsheen et al., “The Intersection Between DNA Damage Response and Cell Death Pathways,” Exp. Onc. 34(3):243-54 (2012), which is hereby incorporated by reference in its entirety). Thus, these results indicate that these cells are primed for apoptosis to occur after multiple days of hormone treatment.

The pathways in this analysis overlap substantially with known drivers of ATC. ATC commonly exhibits mutations to the MAPK and PI3K pathways, the SWI/SNF complex, and DNA repair processes (Landa et al., “Genomic and Transcriptomic Hallmarks of Poorly Differentiated and Anaplastic Thyroid Cancers,” J. Clin. Invest. 126(3):1052-66 (2016), Pozdeyev et al., “Genetic Analysis of 779 Advanced Differentiated and Anaplastic Thyroid Cancers,” Clin. Cancer Res.: Off. J. Amer. Assoc. Cancer Res. 24(13):3059-68 (2018), which is hereby incorporated by reference in its entirety). These are also shown to be altered by TRβ in the analysis herein. The TRβ driven transcriptomic reprogramming is indicative of a reversal of the process of malignancy. Indeed, these changes in gene expression suggest that TRβ is promoting a differentiating effect in the cells, which was demonstrated by the TDS and multiple stemness assays, indicating that TRβ expression may be predictive in determining the aggressiveness of a tumor. The results herein indicate that TRβ reduces the aggressive malignant phenotype of ATC cells.

A significant finding here is that TRβ stimulates the activity of STAT1 in ATC. STAT1 is stimulated by interferons, which have been reported to be tumor suppressive in multiple cancer types (Parker et al., “Antitumour Actions of Interferons: Implications for Cancer Therapy,” Nat. Rev. Cancer. 16(3):131-44 (2016), which is hereby incorporated by reference in its entirety). Interestingly, cytokine-cytokine receptor pathway was predicted to be changed in the thyroids of THRBP^{PV/PV} PTEN^{+/-} mice (Park et al., “Monocyte Recruitment and Activated Inflammation are Associated with Thyroid Carcinogenesis in a Mouse Model,” Am. J Cancer Res. 9(7):1439-53 (2019), which is hereby incorporated by reference in its entirety), a class of interactions that includes interferon-interferon receptor. STAT1 itself may be a clinically important drug target as stimulation of STAT1 activity was sufficient to reduce growth in multiple ATC cell lines. Treatment strategies that focus on activating STAT1-directed signaling may have value for prolonging the lifespan of ATC patients. Several studies have examined the utility of treating thyroid cancer with interferon. Interferon γ reduces proliferation and migration in papillary thyroid cancer (Fallahi et al., “The Paramount Role of Cytokines and Chemokines in Papillary Thyroid Cancer: a Review and Experimental Results,” Immunol Res. 66(6):710-22 (2018), Rotondi et al., “Effect of Interferon-gamma on the Basal and the TNFalpha-Stimulated Secretion of CXCL8 in Thyroid Cancer Cell Lines Bearing Either the RET/PTC Rearrangement Or the BRAF V600e Mutation,” Mediators Inflamm. 2016:8512417 (2016), which is hereby incorporated by reference in its entirety), and interferon α suppresses growth in thyroid cancer cells, including in ATC lines (Selzer et al., “Effects of Type I-Interferons on Human Thyroid Epithelial Cells Derived from Normal and Tumour Tissue,” Naunyn Schmiedebergs Arch Pharmacol. 350(3):322-28 (1994), Lam et al., “In vitro Inhibition of Head and Neck Cancer-Cell Growth by Human Recombinant Interferon-Alpha and 13-cis Retinoic Acid,” Br. J. Biomed. Sci. 58(4):226-9 (2001), which is hereby incorporated by reference in its entirety). Additionally, a phase II trial combined interferon α with doxorubicin and observed a modest response in advanced thyroid cancer patients, however toxicity was high (Argiris et al., “A Phase II Trial of Doxorubicin and Interferon Alpha 2b in Advanced, Non-Medullary Thyroid Cancer,” Invest. New Drugs 26(2):183-8 (2008), which is hereby incorporated by reference in its entirety). Modulation of STAT1 directly to suppress tumor growth may be better tolerated. In general, targeting multiple effectors in the same pathway reduces undesirable side effects in cancer patients (Bayat et al., “Combination Therapy in Combating Cancer,” Oncotarget. 8(23):38022-43 (2017), which is hereby incorporated by reference in its entirety).

The investigation into TRβ signaling was used to elucidate ATC vulnerabilities and uncover a novel regulatory effect of TRβ in ATC by stimulating STAT1. Only recently have targeted therapies in ATC shown success in managing disease burden and progression (Ljubas et al., “A Systematic Review of Phase II Targeted Therapy Clinical Trials in Anaplastic Thyroid Cancer,” Cancers (Basel) 11(7) (2019), which is hereby incorporated by reference in its entirety). These treatment modalities focus primarily upon the mutational landscape of ATC, however epigenetic alterations, including silencing of the tumor suppressor TRβ, have an unrealized potential to inform drug development. Additionally, reversing the epigenetic silencing of TRβ may itself be beneficial. There work herein on TRβ signaling reveals novel functions of TRβ in ATC and the need to investigate STAT signaling in aggressive thyroid cancers.

Finally, the data herein demonstrates that there is value to maintaining euthyroid status in ATC patients. Hypothyroidism is a known consequence of certain treatment regimens (Illouz et al., “Endocrine Side-Effects of Anti-Cancer Drugs: Thyroid Effects of Tyrosine Kinase Inhibitors,” Eur. J. Endocrinol. 171(3):R91-9 (2014), Hartmann, “Thyroid Disorders in the Oncology Patient,” J. Adv. Pract. Oncol. 6(2):99-106 (2015), which is hereby incorporated by reference in its entirety) and treating chemotherapy-induced hypothyroidism may improve clinical outcomes due to stimulation of the tumor suppressive activity of TRβ, illustrating the potential for TRβ as a diagnostic marker.

### Example 6—Selective Activation of TRβ with Agonists Induces Tumor Suppression Signaling and a Less Aggressive Phenotype

Mimetics of T₃ that selectively act as TRβ agonists have been clinically successful for treatment of metabolic disorders, hyperlipidemia, hypercholesterolemia, and non-alcoholic steatohepatitis without the evidence cardiovascular effects mediated by TRα (Trost et al., “The Thyroid Hormone Receptor-beta-selective Agonist GC-1 Differentially Affects Plasma Lipids and Cardiac Activity,” Endocrinology 141:3057-3064 (2000); Grover et al., “Selective Thyroid Hormone Receptor-beta Activation: a Strategy for Reduction of Weight, Cholesterol, and Lipoprotein (a) with Reduced Cardiovascular Liability,” Proc Natl Acad Sci USA 100:10067-10072 (2003), which are hereby incorporated by reference in their entirety). Those in clinical trials include sobetirome (GC-1) (Elbers et al., “Thyroid Hormone Mimetics: the Past, Current Status and Future Challenges,” Current Atherosclerosis Reports 18:14 (2016), which is hereby incorporated by reference in its entirety) with comparable transcriptomic effects to T₃ (Yuan et al., “Identical Gene Regulation Patterns of T3 and Selective Thyroid Hormone Receptor Modulator GC-1,” Endocrinology 153:501-511 (2012), which is hereby incorporated by reference in its entirety), and MGL-3196 (Kelly et al., “Discovery of 2-[3,5-dichloro-4-(5-isopropyl-6-oxo-1,6-dihydropyridazin-3-yloxy)phenyl]-3,5-dioxo-2,3,4,5-tetrahydro[1,2,4]triazine-6-carbonitrile (MGL-3196), a Highly Selective Thyroid Hormone Receptor Beta Agonist in Clinical Trials for the Treatment of Dyslipidemia,” J Med Chem 57:3912-3923 (2014), which is hereby incorporated by reference in its entirety). Both agonists bind to the TRβ ligand-binding domain with high affinity and specificity.

Selective TRβ agonism with T₃, sobetirome (also known as GC-1) and derivatives, MGL-3196, and other TRβ specific molecules activates TRβ1 signaling in tumor cells with endogenous expression of TRβ in both differentiated, less aggressive thyroid and breast cancer cells, and undifferentiated, aggressive thyroid and breast cancer cells. A greater effect of agonism of TRβ corresponds to the level of endogenous levels of TRβ and/or duration of treatment.

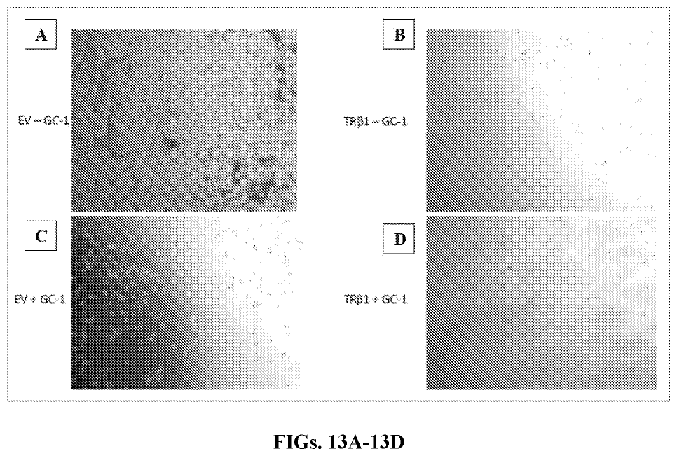

To determine the effect that TRβ agonism would have in anaplastic thyroid cancer cells, SW1736 cells were transduced with a lentiviral vector (EV) or a lentiviral vector containing the TRβ cDNA (TRβ). Growth in the absence of TRβ (FIG. 13A) and presence of TRβ (FIG. 13B) reveals that re-expression of TRβ in ATC cells (SW1736) induces cell death. Activation of endogenous TRβ with GC-1 (10⁻⁸ M) induces ATC cell death; an effect that is amplified with higher levels of TRβ (FIG. 13D) achieved by lentiviral transduction. Thus, GC-1 induces ATC cell death and amplifies the effect of expression of TRβ.

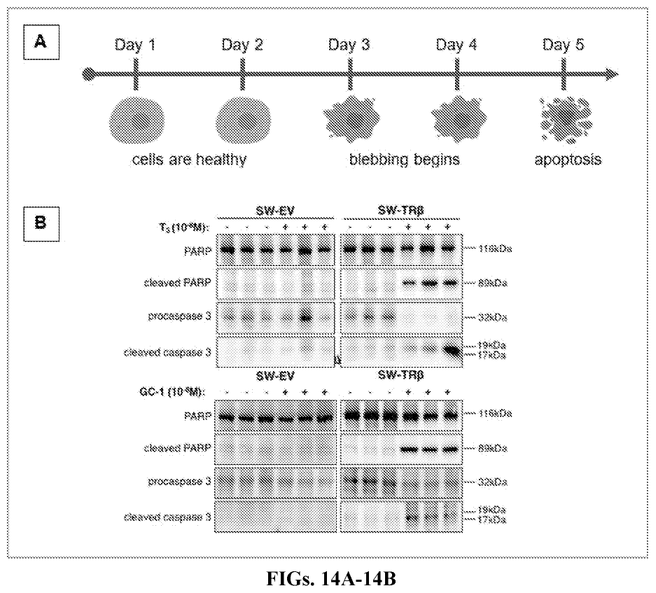

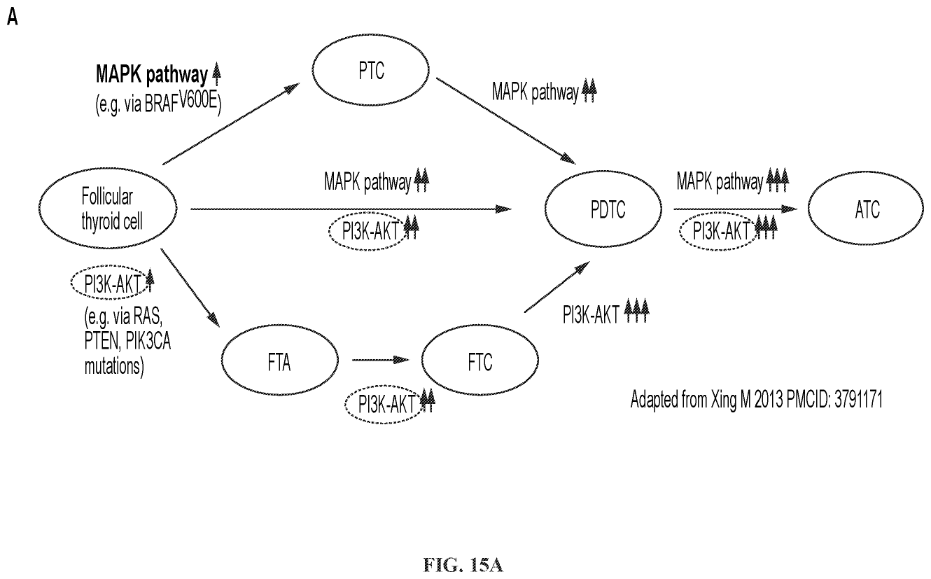

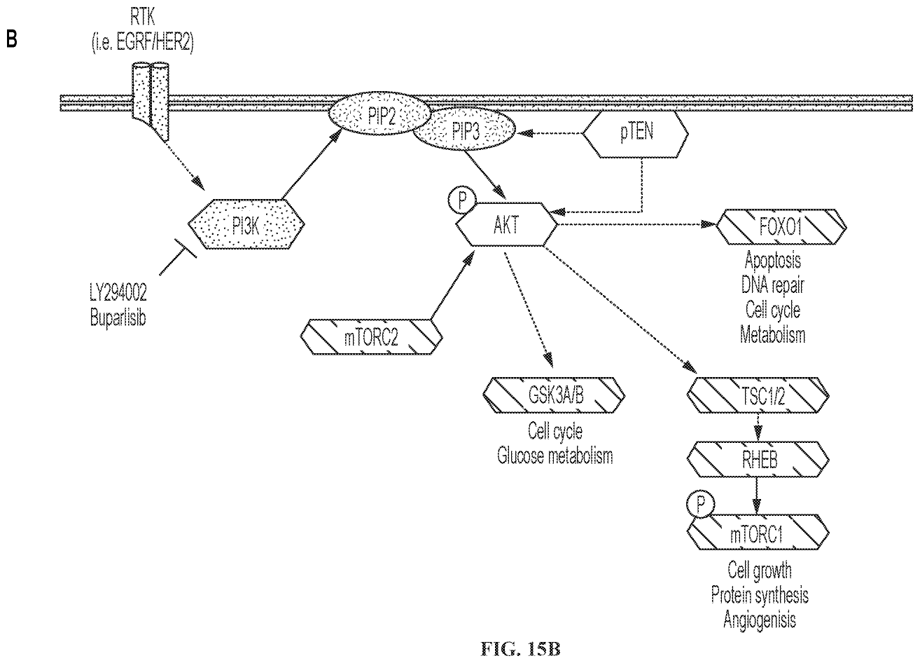

TRβ agonism with GC-1 also induces apoptotic signaling in cells with TRβ re-expressed. FIG. 14 shows that activation of TRβ by ligand triiodothyronine (T₃) or selective agonist sobetirome (GC-1) induces apoptosis in anaplastic thyroid cancer cells (SW1736) through the JAK1-STAT1 signaling pathway. TRβ was over-expressed in SW1736 cells by lentiviral transduction (SW-TRβ) and compared with cells transduced with an empty vector control (SW-EV). Cells were treated with either T₃ or GC-1 for five days. Apoptosis (schematically represented in FIG. 14A) was observed in treated cells in which TRβ levels are detected. In both treatments, TRβ induces apoptosis in part through JAK1-STAT1 signaling as reflected by cleaved PARP and caspase illustrated in the immunoblots of FIG. 14B. No cell phenotypic changes or signaling changes were observed in normal-like NthyOri thyroid cells (not shown).

### Example 7—Activated TRβ Increases the Efficacy of Therapeutic Inhibitors, Selective Intracellular Signaling Pathways Regulated by TRβ

Increased PI3K-Akt pathway activity is a hallmark of thyroid, breast, and other solid tumors (FIG. 15A). To date, PI3K inhibitors have limited efficacy and are frequently toxic. Currently, the PI3K inhibitor buparlisib is in Phase II clinical trials for TNBC and lymphomas, and LY294002, an analogue of buparlisib, has been tested experimentally but not clinically (FIG. 15B).

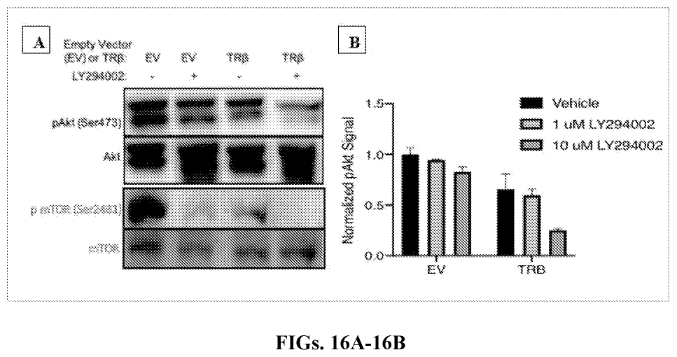

To determine whether expression of TRβ would enhance the efficacy of the PI3K inhibitor LY294002, SW1736 ATC cells were transduced with either empty vector (EV) or vector with TRβ (TRβ), and treated with LY294002 for 24 hr in the presence of ligand T₃ (10⁻⁸ M). PI3K activity is reflected by detection of target proteins, phosphorylated Akt (pAkt) and phosphorylated mTOR (p mTOR). The presence of TRβ decreases PI3K activity and further increases the effectiveness of LY294002 as reflected by decreased pAkt compared with total Akt and decreased p mTOR compared with total mTOR detected by immunoblot (FIG. 16A). Quantitation of the immunoblot is shown in FIG. 16B.

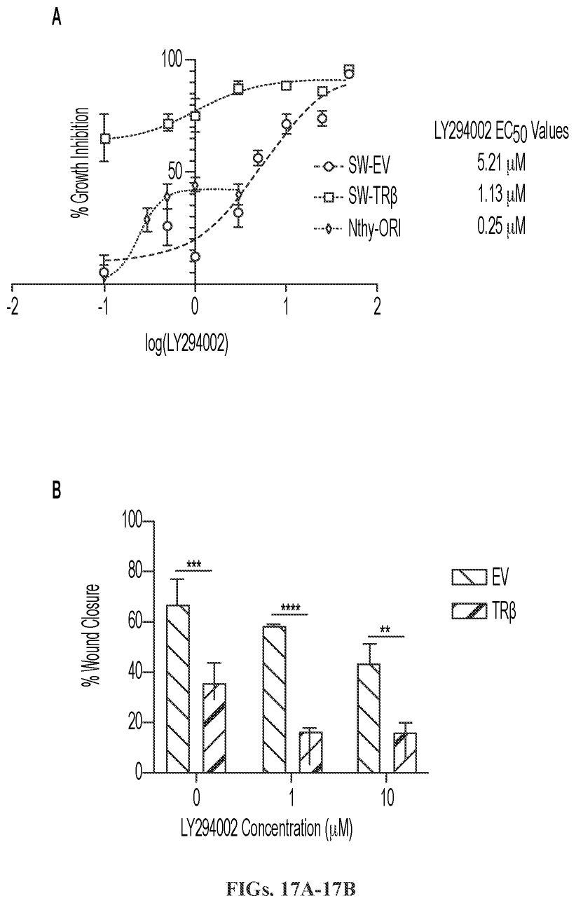

TRβ also enhances PI3K inhibitor LY294002 inhibition of ATC cell growth and cell migration. The presence of liganded TRβ (SW-TRβ) increases the efficacy of LY294002 in ATC cells compared to control cells lacking TRβ re-expression (SW-EV) with an EC₅₀ of 1.13 compared with 5.21 respectively (FIG. 17A). LY294002 induces a 50% reduction in growth of normal-like thyroid cells NthyOri in which TRβ is expressed. The presence of liganded TRβ enhances LY294002 inhibition of ATC cell migration (FIG. 17B). Liganded TRβ suppresses ATC cell growth and migration as a tumor suppressor and critically enhances the effectiveness of a potential therapeutic agent.

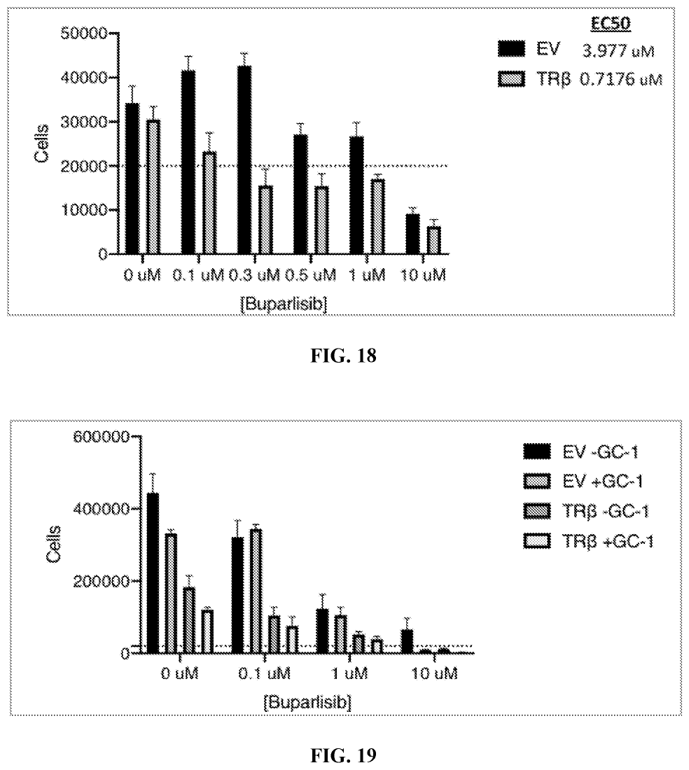

The presence of TRβ also increases the efficacy of buparlisib, another known inhibitor of PI3K activity. SW1736 cells transduced with lentiviral vector (EV) or lentiviral vector containing TRβ (TRβ) were treated with buparlisib (0 μM, 0.1 μM, 1 μM, or 10 μM) for 24 hours. Buparlisib alone causes a concentration dependent decrease in cell viability (EV) as shown in FIG. 18. However, the presence of TRβ (TRβ) increases the sensitivity of cells to buparlisib resulting in a significantly reduced EC₅₀ as also shown in FIG. 18. As shown herein (Example 3) the re-expression of TRβ results in an increase in ATC differentiation and a less aggressive phenotype.

Activation of TRβ with selective agonist GC-1 also enhances buparlisib inhibition of ATC cell growth. SW-EV or SW-TRβ cells were treated with GC-1 (10⁻⁸M) for 24 hours in the absence or presence of increasing concentrations of buparlisib. As noted, TRβ (TRβ) decreases cell growth in the absence of buparlisib. The addition of GC-1 with TRβ further reduces cell growth as shown in FIG. 19. GC-1 alone (EV+GC-1) inhibits cell growth through the low levels of endogenous TRβ. However, TRβ and GC-1 increase the sensitivity to buparlisib at 0.1 and 1 μM as shown in FIG. 19. Buparlisib at 10 μM is toxic.

### Materials and Methods for Examples 8-10

Culture of thyroid cell lines. Anaplastic thyroid cancer cell lines (SW1736, 8505C, OCUT-2, and KTC-2) were cultured in RPMI 1640 growth media with L-glutamine (300 mg/L), sodium pyruvate and nonessential amino acids (1%) (Corning Inc, Corning, N.Y., USA), supplemented with 10% fetal bovine serum (Thermo Fisher Scientific, Waltham, Mass., USA) and penicillin-streptomycin (200 IU/L) (Corning) at 37° C., 5% CO₂, and 100% humidity. SW1736 cells were modified by lentiviral transduction as recently described with either an empty vector (SW-EV) or to overexpress TRβ (SW-TRβ). SW-EV and SW-TRβ were grown in the above conditions with the addition of 1 μg/ml puromycin (Gold Bio, St Louis, Mo., USA). SW1736 and KTC-2 were authenticated by the Vermont Integrative Genomics Resource at the University of Vermont using short tandem repeat profiles and Promega GenePrint10 System (SW1736, May 2019; KTC-2, October 2019). 8505C and OCUT2 were authenticated by the University of Colorado using short tandem repeat profiles (8505C, June 2013; OCUT-2, June 2018).

Growth Assays. Cell growth was measured by cell counting at discrete points. Cells were seeded in 12-well plates and growth optimized. Cells were treated with GC-1, Buparlisib, Alpelisib, Palbociclib, or Sorafenib to establish time and concentration effects of the select agents on cell growth. Cell viability was determined by a Sulforhodamine B assay (Abcam, ab235935) following the manufacturer's protocol. In brief, ATC cells were plated in 96-well clear flat-bottom plates at a density of 5,000 cells per well. Cells were fixed, stained, and then imaged using a plate reader according to manufacturer's instructions.

Migration Assay. Cell migration was determined by wound healing assay. Cells were plated and allowed to grow to 100% confluency. Four hours prior to scratching, cells were treated with 10 mg/ml Mitomycin C. A scratch was performed with a P1000 pipette tip and debris was washed away with PBS. Media was supplemented with GC-1 (10⁻⁸M) without and with 0.5 μM Buparlisib, 0.5 μM Alpelisib, 1 nM Palbociclib, or 5 μM Sorafenib. Images were obtained at 0, 16, 24, 48, and 72 hours or until 100% wound closure, depending on the cell type. The wound closure was measured using the ImageJ macro “Wound Healing Tool” (https://github.com/MontpellierRessourcesImagerie/imagej_macros_and_scripts/wiki/Wound-Healing-Tool). Percent closure was calculated relative to the area of the initial scratch.

Tumorsphere Assay. Cancer stem cell tumorspheres formed from ATC cells (thyrospheres) were used to assess self-renewal and sphere-forming efficiency. For generating thyrospheres, adherent monolayer cells were dissociated with Trypsin-EDTA, and single cells were moved to round-bottom ultra-low attachment 24-well plates at a density of 1000 cells/well (Corning). Thyrospheres were cultured in RPMI 1640 growth media supplemented with 20 ng/mL each of epidermal growth factor (EGF) and fibroblastic growth factor (FGF) (Gold Bio). Where indicated, adherent cells were treated with 0.5 μM Buparlisib, 0.5 μM Alpelisib, 1 nM Palbociclib, 5 μM Sorafenib, or vehicle for 72 hours prior to thyrosphere-forming assay. Thyrospheres were then cultured in the presence or absence of GC-1 (10⁻⁸M) to evaluate the effects of liganded TRβ on thyrosphere growth alone or subsequent to a therapeutic agent treatment. Thyrospheres grew for seven days and were then counted with an inverted microscope.

Immunoblot Analysis. Proteins were isolated from whole cells in lysis buffer (20 mM Tris-HCl [pH 8], 137 mM NaCl, 10% glycerol, 1% Triton X-100, and 2 mM EDTA) containing Protease Inhibitor Cocktail 78410 (Thermo Fisher Scientific). Proteins were resolved by polyacrylamide gel electrophoresis on 10% sodium dodecyl sulfate gels EC60752 (Thermo Fisher Scientific) and immobilized onto nitrocellulose membranes (GE Healthcare, Chicago, Ill., USA) by electroblot (Bio-Rad Laboratories, Hercules, Calif., USA). Specific proteins were detected by immunoblotting with the indicated antibodies (Table 4); immunoreactive proteins were detected by enhanced chemiluminescence (Thermo Scientific) on a ChemiDoc XRS+ (Bio-Rad Laboratories).

RNA Extraction and Quantitative Real-Time PCR (qRT-PCR). Total RNA was extracted using RNeasy Plus Kit (Qiagen) according to manufacturer's protocol. cDNA was then generated using the 5×RT Master mix (ABM, Vancouver, Canada). mRNA expression was quantified by qRT-PCR using 2× SuperGreen Master mix (ABM, Vancouver, Canada) on a QuantStudio 3 real-time PCR system (Applied Biosystems). Fold change in gene expression compared to endogenous controls was calculated using the ddCT method. Primer sequences are indicated in Table 5.

Statistics. All statistical analyses were performed using GraphPad Prism software. Paired comparisons were by T-test and group comparisons were made by a 2-way ANOVA followed by a Tukey multiple comparisons test or Sidak's multiple analyses where indicated. Data are represented as mean±standard deviation. Significance is indicated * p<0.05. Area under the curve (AUC) at the 95th confidence interval was used to evaluate statistical differences in growth and migration assays.

### Example 8—Sobetirome, GC-1, Blocks the Tumorigenic Phenotype and Cancer Stem Cell Growth in ATC Cells Transduced with TRβ

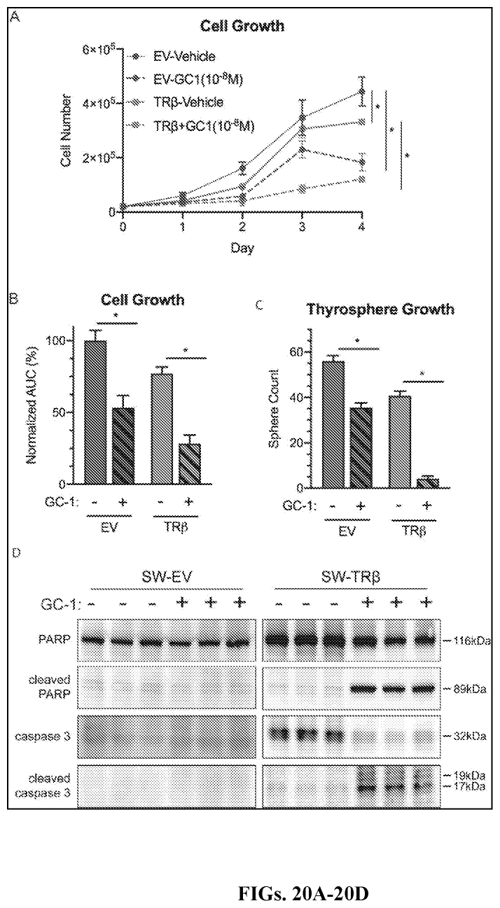

It has previously been demonstrated that triiodothyronine (T₃, 10⁻⁸M) treatment of SW1736 cells in which TRβ1 is re-expressed (SW-TRβ) induced a tumor suppression transcriptomic program and reduced cell growth with less effect on cells transduced with empty vector (SW-EV) with low levels of endogenous TRIS (Bolf et al., “Thyroid Hormone Receptor Beta Induces a Tumor-Suppressive Program in Anaplastic Thyroid Cancer,” Mol Cancer Res. 18(10):1443-1452 (2020), which is hereby incorporated by reference in its entirety). In the present studies, the effect of GC-1 (10⁻⁸M) to alter the ATC phenotype and cancer cell stemness was tested. It was found that GC-1 induced a significant decrease in cell growth in 4 days (FIG. 20A) with a 50% reduction in SW-EV and 75% reduction in SW-TRβ indicating potent activation of endogenous TRβ1 (FIG. 20B).

As cancer stem cells are considered to be responsible for tumor initiation and tumor recurrence after chemotherapy, targeting these cells is a key for successful treatment. Thus a critical foundation is to establish whether thyroid cancer stem cells are responsive to GC-1. Thyrosphere formation is robust in the ATC cell line SW-EV. GC-1 induced a significant reduction in growth (FIG. 20C). Re-expression of TRβ1 (SW-TRβ1) reduces thyrosphere growth and the addition of GC-1 induces a greater than 80% reduction in thyrosphere formation (FIG. 20C). These results indicate that GC-1 alone can activate TRβ1 to reduce the thyroid cancer stem cell population.

Apoptotic signaling is a key factor in mediating ligand activated TRβ1 modification of the ATC aggressive phenotype (Bolf et al., “Thyroid Hormone Receptor Beta Induces a Tumor-Suppressive Program in Anaplastic Thyroid Cancer,” Mol Cancer Res. 18(10):1443-1452 (2020), which is hereby incorporated by reference in its entirety). Thus, the effects of GC-1 on PARP and caspase 3 cleavage were assessed by immunoblot as markers of late-stage apoptosis (FIG. 20D). GC-1 significantly increased the presence of cleaved PARP and cleaved caspase 3 in SW-TRβ consonant with an increase in apoptotic signaling.

These results indicate that GC-1 can activate TRβ1 to reduce the aggressive phenotype in transduced ATC cells with TRβ1 re-expressed and recapitulate the effects seen with T3 at comparable concentrations.

### Example 9—GC-1 Slows Growth of Parental Cells

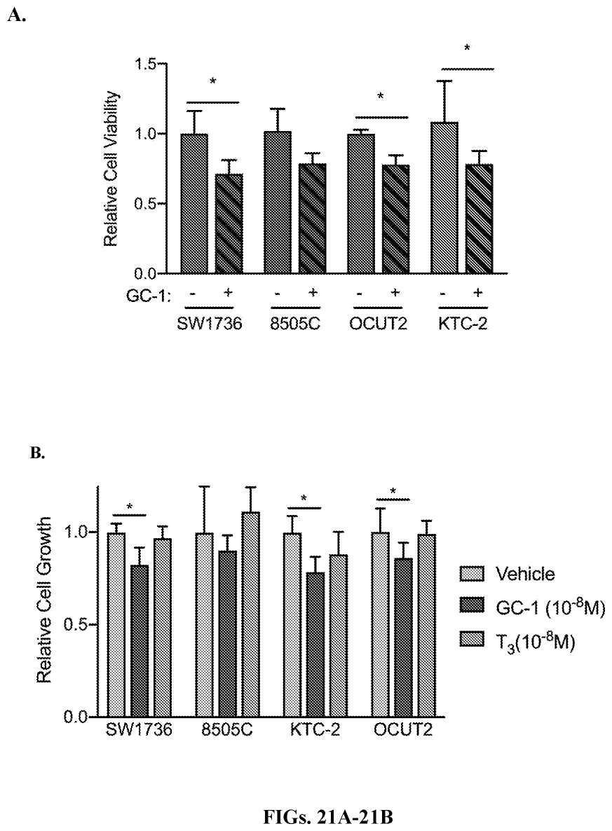

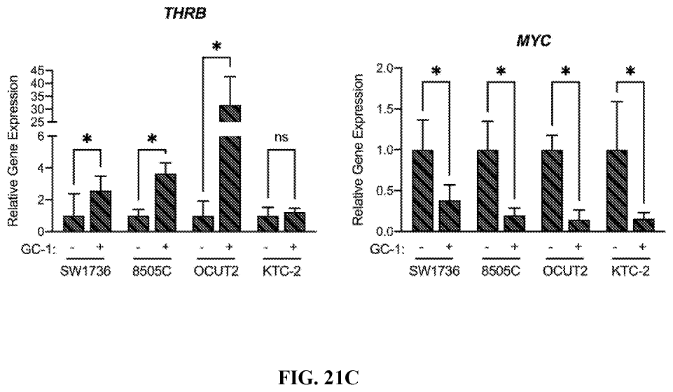

That GC-1 can reduce the aggressive phenotype in transduced ATC cells affirms ligand effective activation of TRβ1. However, it is critical to assess whether agonist selective activation of endogenous TRβ1 induces similar effects in non-transformed ATC cell lines with diverse mutational backgrounds and low levels of endogenous TRβ1 (Landa et al., “Genomic and Transcriptomic Hallmarks of Poorly Differentiated and Anaplastic Thyroid Cancers,” The Journal of Clinical Investigation 126(3):1052-1066 (2016), which is hereby incorporated by reference in its entirety). SW1736, 8505C, OCUT2, and KTC-2 cells all harbor BRAFV600E and TERT driver mutations. SW1736 and 8505C have additional TP53 mutations. SW1736 has an additional TSHR mutation, and OCUT2 has a PIK3CA mutation. Treatment of each of these cell lines with GC-1 (10-8M) for four days yielded a significant decrease in cell viability for SW1736, OCUT2C, and KTC-2 and a reduction but not significant in 8505C at that time point (FIG. 21A). However, treatment of the cells with GC-1 (10⁻⁸M) significantly reduced cell growth reflective of the doubling times of the individual cell lines (FIG. 21B). The effectiveness of GC-1 to reduce cell growth at different concentrations was further evaluated, and it was found that lower but significant reduction in cell growth was noted at 10-9M and no significant reduction occurred at 10-10M. Higher concentrations cause cell toxicity, therefore GC-1 was used at 10-8M for further experiments.

Since ligand activated TRβ1 induces a tumor suppression transcriptomic program in ATC cells, we measured key markers of ligand regulated gene expression in response to GC-1 treatment. MYC is a well-characterized factor associated with malignant transformation and metastases of solid tumors including thyroid cancer (Sanjari et al., “Enhanced Expression of Cyclin D1 and C-myc, a Prognostic Factor and Possible Mechanism for Recurrence of Papillary Thyroid Carcinoma,” Sci Rep. 10(1):5100 (2020), which is hereby incorporated by reference in its entirety). It is also a TRβ1 target gene with a defined thyroid hormone response element mediating suppression (Perez-Juste et al., “An Element in the Region Responsible for Premature Termination of Transcription Mediates Repression of c-myc Gene Expression by Thyroid Hormone in Neuroblastoma Cells,” J Biol Chem. 275(2):1307-1314 (2000), which is hereby incorporated by reference in its entirety). GC-1 induced a significant suppression of MYC expression, a known therapeutic target. Conversely, GC-1 significantly increased TRβ1 expression in all four cell lines after 24 hours (FIG. 21D). GC-1 activation of endogenous TRβ1 suppressed a key tumor promoter, MYC, while increasing the expression or the endogenous tumor suppressor TRβ1 in ATC cell lines with diverse genetic backgrounds.

### Example 10—GC-1 Increases the Efficacy of Selected Therapeutic Agents

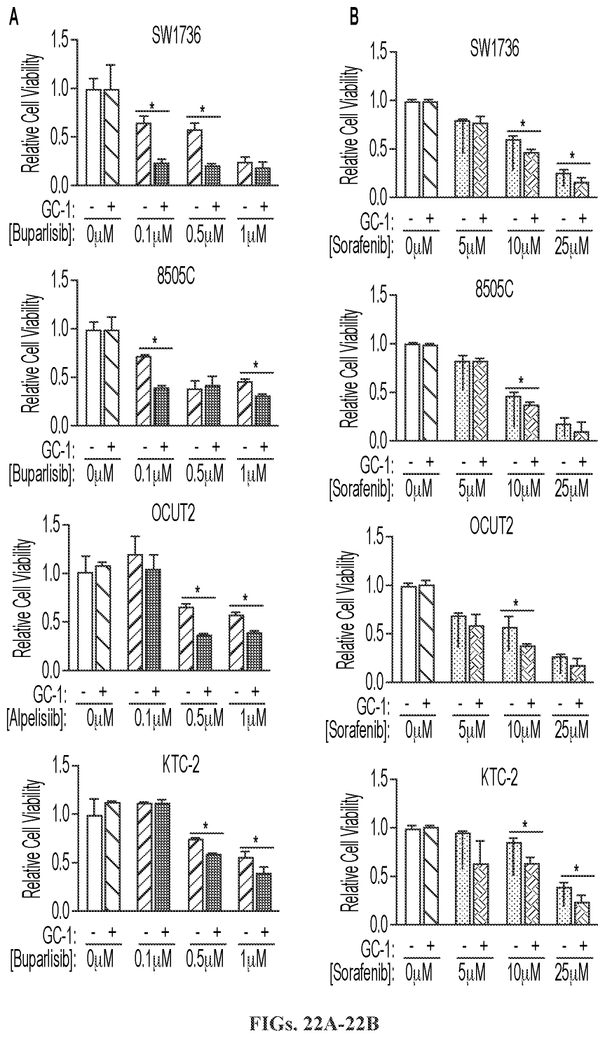

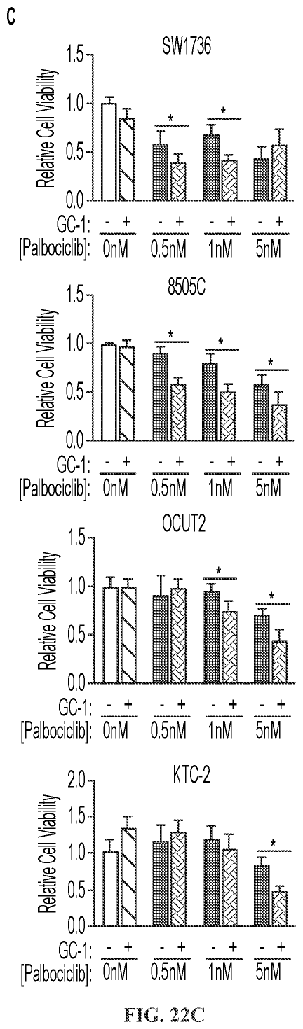

As agonist activation of TRβ1 can induce a tumor suppression program and reduce the aggressive tumor phenotype in ATC cells, a critical question is whether GC-1 can enhance the efficacy of therapeutics that target selective pathways. Cells were treated for 3 days with increasing concentrations of PI3K inhibitor Buparlisib or Alpelisib for PI3K mutant OCUT2 cells; tyrosine kinase inhibitor Sorafenib; cell cycle inhibitor Palbociclib, with or without GC-1 and cell viability determined (FIG. 22). Each of the therapeutic agents decreased cell viability in 3 days while GC-1 did alter cell viability in that time period. However, the combination of GC-1 and any of the agents significantly increased the efficacy of these agents at lower concentrations.

### Example 11—GC-1 Enhances the Effects of Therapeutic Agents on Cell Migration

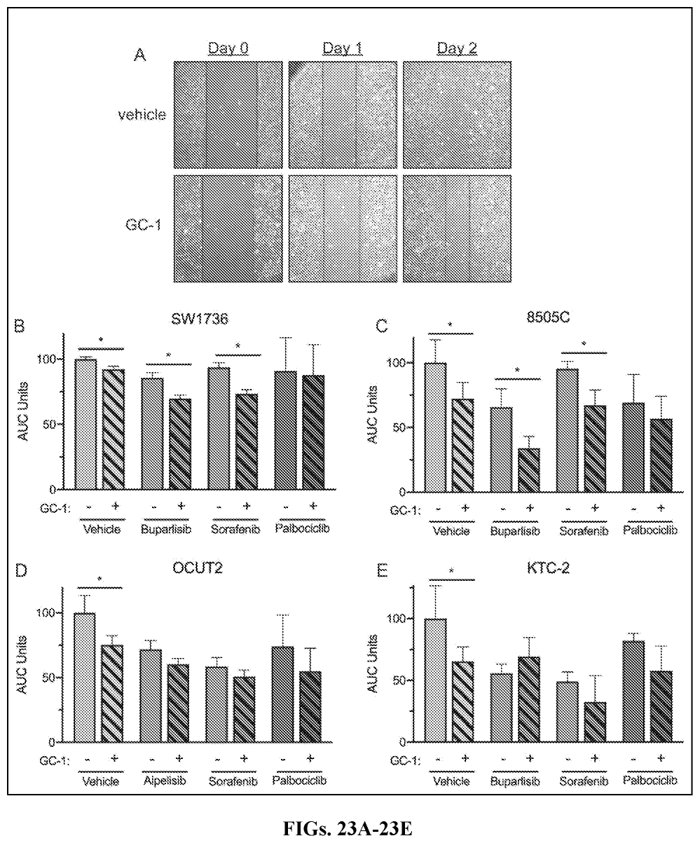

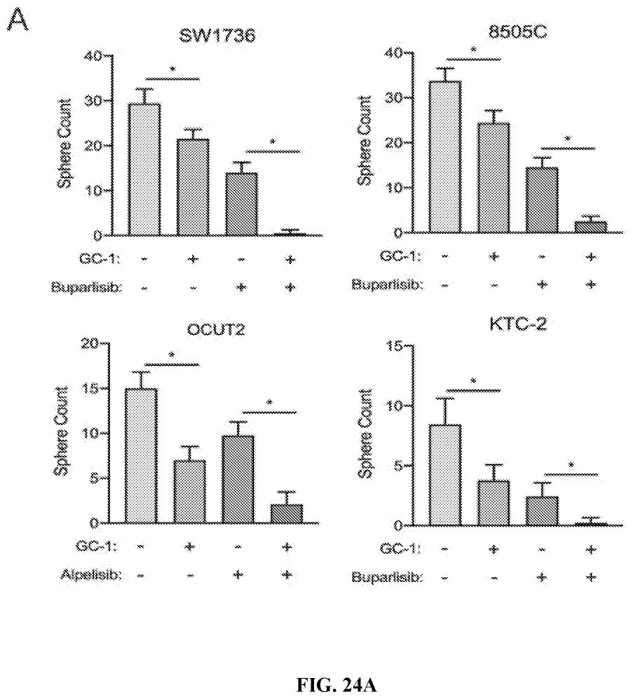

In addition to reducing cell viability, many therapeutics target pathways involved in tumor cell migration. To further evaluate the ability of GC-1 to enhance the efficacy of select therapeutics, cells were treated for indicated inhibitor at concentrations determined to be effective based on the cell viability assays or vehicle and migration was observed by assessing wound closure (FIG. 22). Each inhibitor was able to successfully prevent wound closure and this effect was increased when each of the inhibitors were combined with GC-1, with the exception of Buparlisib in the KTC-2 cells (FIG. 22B-22E). Notably, GC-1 alone was able to prevent wound closure of all tumor cell lines with similar success as each inhibitor alone (FIG. 23A-23E). These findings are similar to what was previously observed in experiments using endogenous thyroid hormone, T₃, indicating GC-1 is a suitable alternative for TRβ1 activation and mitigation of malignant phenotype.

### Example 12—GC-1 Blocks Thyrosphere Outgrowth and Increases the Efficacy of Therapeutic Agents

Current treatment options for ATC primarily target hyperproliferative cells, but aggressive cancers like ATC are enriched for long-lived cancer stem cells that are thought to be a major mechanism for therapy resistance and tumor recurrence. The signaling pathways that govern stemness and proliferation are separate and frequently oppose each other. It has previously been demonstrated that ligand-activated TRβ1 regulates genes related to stemness in ATC cells and that GC-1 could block thyrosphere outgrowth in our modified ATC cells (FIG. 20D). Therefore, the hypothesis that GC-1 could prevent thyrosphere outgrowth after an initial treatment with an inhibitor that targets proliferation was tested.

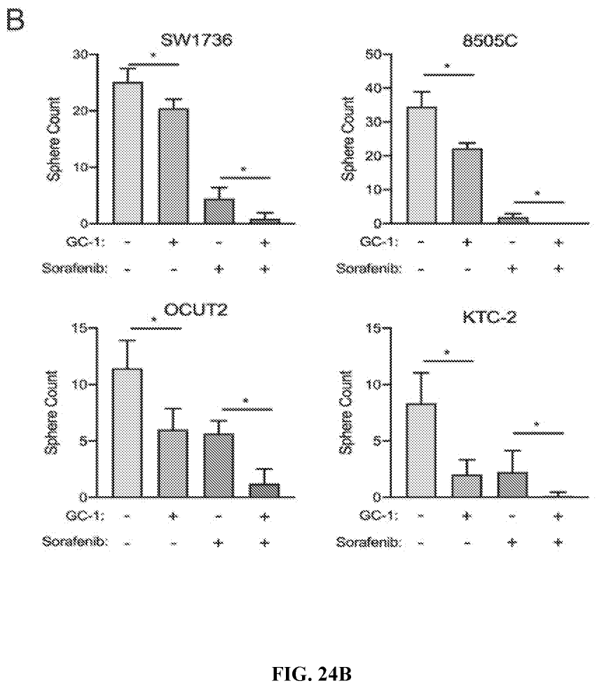

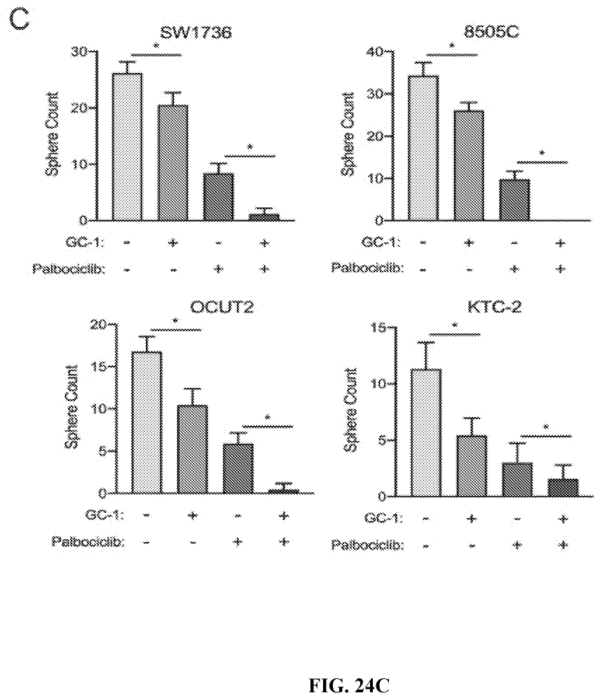

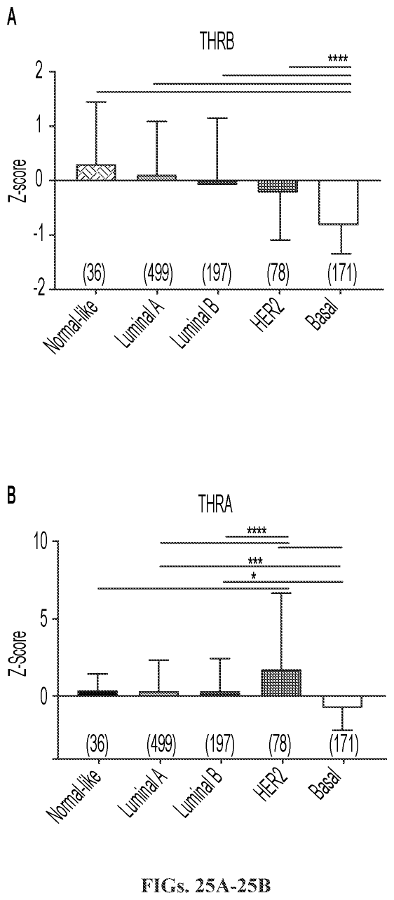

ATC cells were treated with the indicated inhibitor or vehicle for three days, followed by re-plating in non-adherent serum-free conditions. It was again observed that GC-1 alone could reduce thyrosphere formation in unmodified ATC cell lines (FIG. 24A-24C). None of the inhibitors could completely prevent thyrosphere outgrowth when used alone, however Sorafenib was the most effective overall (FIG. 24B). Strikingly, the addition of GC-1 upon removal of each inhibitor blocked nearly all thyrosphere outgrowth. This effect was consistent across all four cell lines and all three inhibitors used in this experiment. This result suggests that GC-1 may be particularly effective at blocking cancer stem cell expansion.

### Discussion of Examples 8-10:

Nuclear receptors (NRs) are master regulators of growth and differentiation and are increasingly recognized as diagnostic and therapeutic targets in thyroid, breast, and other hormone-dependent cancers. Yet, the actions of non-steroidal NRs in these cancers are not well characterized (Doan et al., “Emerging Functional Roles of Nuclear Receptors in Breast Cancer,” J Mol Endocrinol. 58(3):R169-R190 (2017), which is hereby incorporated by reference in its entirety). There is compelling evidence that loss of expression of TRβ, a member of the thyroid hormone receptor (TR) family, through genomic modifications and epigenetic silencing is characteristic of thyroid, breast and other endocrine-related tumors (Silva et al., “Expression of thyroid hormone receptor/erbA genes is altered in human breast cancer,” Oncogene. 21:4307 (2002); Kim et al., “Thyroid Hormone Receptors and Cancer,” Vol 1830. Biochim Biophys Acta 2013:3928-3936; Aranda et al., “Thyroid receptor: Roles in Cancer,” Trends Endocrinol Metab. 20(7):318-324 (2009); Jezequel et al., “bc-GenExMiner: an Easy-to-use Online Platform for Gene Prognostic Analyses in Breast Cancer,” Breast Cancer Res Treat 131(3):765-775 (2012); Jezequel et al., “bc-GenExMiner 3.0: New Mining Module Computes Breast Cancer Gene Expression Correlation Analyses,” Database (Oxford). 2013:bas060 (2013); Wojcicka et al., “Epigenetic Regulation of Thyroid Hormone Receptor Beta in Renal Cancer,” PLoS One 9(5):e97624 (2014); Park et al., “Oncogenic Mutations of Thyroid Hormone Receptor β,” Oncotarget. 6(10):8115-8131 (2015), which are hereby incorporated by reference in their entirety).

Multiple lines of evidence demonstrate the tumor suppressive activity of TRβ in thyroid cancer. Numerous pre-clinical studies demonstrate the potent anti-tumorigenic effect of robust expression of the TRβ gene. Re-expression of silenced TRβ using demethylating agents delays thyroid tumor progression in vivo (Kim et al., “Reactivation of the Silenced Thyroid Hormone Receptor Beta Gene Expression Delays Thyroid Tumor Progression,” Endocrinology 154(1):25-35 (2013), which is hereby incorporated by reference in its entirety), mice expressing a c-terminal frameshift of TRβ (ThrbPV) spontaneously develop follicular thyroid cancer (Suzuki et al., “Mice with a Mutation in the Thyroid Hormone Receptor Beta Gene Spontaneously Develop Thyroid Carcinoma: a Mouse Model of Thyroid Carcinogenesis,” Thyroid 12(11):963-969 (2002), which is hereby incorporated by reference in its entirety), and ThrbPV/PV KrasG12D mice develop thyroid tumors with features of dedifferentiated thyroid cancer (Zhu et al., “Synergistic Signaling of KRAS and Thyroid Hormone Receptor Beta Mutants Promotes Undifferentiated Thyroid Cancer Through MYC Up-regulation,” Neoplasia 16(9):757-769 (2014), which is hereby incorporated by reference in its entirety). The studies in anaplastic thyroid cancer cells revealed that activation of TRβ induces a tumor suppression transcriptomic program and reduces the aggressive phenotype (Carr et al., “Thyroid Hormone Receptor-beta (TRbeta) Mediates Runt-Related Transcription Factor 2 (Runx2) Expression in Thyroid Cancer Cells: A Novel Signaling Pathway in Thyroid Cancer,” Endocrinology 157(8):3278-3292 (2016); Bolf et al., “Thyroid Hormone Receptor Beta Induces a Tumor-Suppressive Program in Anaplastic Thyroid Cancer,” Mol Cancer Res. 18(10):1443-1452 (2020), which is hereby incorporated by reference in its entirety). Selective activation of TRβ cannot be achieved through standard thyromimetics.

Agonists have been developed to target tissue specific expression of the most abundant isomers (Davis et al., “Bioactivity of Thyroid Hormone Analogs at Cancer Cells,” Front Endocrinol (Lausanne) 9:739 (2018), which is hereby incorporated by reference in its entirety). Selective activation of TRβ in pre-clinical and clinical studies have yielded exciting results with great promise for novel interventions. Patients who were rendered hypothyroxinemic and treated with T3 alone had longer survival with end-stage solid tumors (Hercbergs et al., “Medically Induced Euthyroid Hypothyroxinemia May Extend Survival in Compassionate Need Cancer Patients: an Observational Study,” Oncologist 20(1):72-76 (2015), which is hereby incorporated by reference in its entirety) and T3 treatment enhanced chemosensitivity (Huang et al., “Implication from Thyroid Function Decreasing During Chemotherapy in Breast Cancer Patients: Chemosensitization Role of Triiodothyronine,” BMC Cancer 13:334 (2013), which is hereby incorporated by reference in its entirety). Following tyrosine kinase inhibitor therapy, patients demonstrated improved survival with treatment of hypothyroidism (Lechner et al., “Hypothyroidism During Tyrosine Kinase Inhibitor Therapy Is Associated with Longer Survival in Patients with Advanced Nonthyroidal Cancers,” Thyroid: official journal of the American Thyroid Association 28(4):445-453 (2018), which is hereby incorporated by reference in its entirety). Mimetics of T3 that selectively act as TRβ agonists have been clinically successful for treatment of metabolic disorders, hyperlipidemia, hypercholesterolemia and non-alcoholic steatohepatitis without the cardiovascular effects mediated by TRβ (Trost et al., “The Thyroid Hormone Receptor-beta-selective Agonist GC-1 Differentially Affects Plasma Lipids and Cardiac Activity,” Endocrinology 141(9):3057-3064 (2000); Grover et al., “Selective Thyroid Hormone Receptor-beta Activation: a Strategy for Reduction of Weight, Cholesterol, and Lipoprotein (a) with Reduced Cardiovascular Liability,” Proc Natl Acad Sci USA 100(17):10067-10072 (2003), which are hereby incorporated by reference in their entirety). Those in clinical trials include TRβ selective sobetirome (GC-1) (Elbers et al., “Thyroid Hormone Mimetics: the Past, Current Status and Future Challenges,” Curr Atheroscler Rep. 18(3):14 (2016), which is hereby incorporated by reference in its entirety) with comparable transcriptomic effects of T3 (Yuan et al., “Identical Gene Regulation Patterns of T3 and Selective Thyroid Hormone Receptor Modulator GC-1,” Endocrinology 153(1):501-511 (2012), which is hereby incorporated by reference in its entirety).

The findings here demonstrate that GC-1 alone, decreases the ATC aggressive phenotype in transduced cells in which TRβ is re-expressed and parental cell lines with diverse genetic backgrounds. These results emphasize the generalizability of GC-1 to activate a TRβ tumor suppression program. This observation is particularly critical in that GC-1 has already been evaluated in Phase II clinical trial for the treatment of metabolic disorders at comparable dose ranges. GC-1 alone does not have the deleterious toxic side effects as the more commonly used cancer therapeutic agents.

In anaplastic thyroid cancer tumors, several genetic alterations and disrupted signaling pathways are usually found. Therefore the treatment aim is to inhibit or disrupt several pathways. Combination strategies use therapies that target cell survival, proliferation and typically target MAPK, PI3K, and cell cycle pathways (Molinaro et al., “Anaplastic Thyroid Carcinoma: from Clinicopathology to Genetics and Advanced Therapies,” Nature Reviews Endocrinology 13:644 (2017); Alobuia et al., “Contemporary Management of Anaplastic Thyroid Cancer,” Curr Treat Options Oncol. 21(10):78 (2020), which are hereby incorporated by reference in their entirety). There are many therapeutics that are in use in combination or alone including multi-kinase inhibitors, epigenetic modulators, apoptosis-inducing agents, and others. However, to date, none of been successful in long-term treatment. Development of resistance and toxicity are limitations to the use. In this study, representative drugs were tested that targeted the PI3K, MAPK, and cell cycle pathways to assess whether GC-1 could alter the effectiveness of these drugs on their targets. Since it has been demonstrated that activation of TRβ in ATC represses the PI3K pathway through genomic mechanisms (Bolf et al., “Thyroid Hormone Receptor Beta Induces a Tumor-Suppressive Program in Anaplastic Thyroid Cancer,” Mol Cancer Res. 18(10):1443-1452 (2020), which is hereby incorporated by reference in its entirety), GC-1 with either Buparlisib or Alpelisb would be suppressing PI3K activity in concert. However, TRβ does not directly impact MAPK or cell cycle signaling through transcriptomic modulation so GC-1 in combination with either Sorafenib or Palbociclib demonstrated concordant effects. In all studies, GC-1 reduced the effective concentrations required to observe a reduction in cell viability. Not surprisingly, not all effects were noted for cell migration as these events are mediated by distinct cell signaling pathways modulated by TRβ.

The most important observation is the impact of GC-1 alone and in combination with the drugs on cancer stem cell growth in thyrosphere formation. This assay is used to determine the potential effectiveness of therapeutics. In all ATC cell types, GC-1 alone significantly reduced thyrosphere formation. Remarkably, GC-1 in combination with any of the drug treatments dramatically reduced the thyrosphere formation. The concentrations of the drugs tested in the thyrospheres were 10-20 fold lower than maximal doses as determined in FIG. 21. In all ATC cell lines, the thyrospheres were minimally detected if at all.

The findings in this study, demonstrate for the first time, that selective activation of TRβ with Sobetirome, GC-1, alone can reduce the aggressive phenotype, reduce the cancer stemness in anaplastic thyroid cancer cells and increase the sensitivity of these cells to therapeutic agents. These studies provide the foundation for further establishing the broad impact of GC-1 alone and in combination with various therapeutics in mitigating toxicity and the development of resistance to treatment.

### Example 13—Activation of TRβ with Isoform Selective Agonists Inhibits Tumor Cell Growth and Induces Mammosphere (Cancer Stem Cell) Suppression

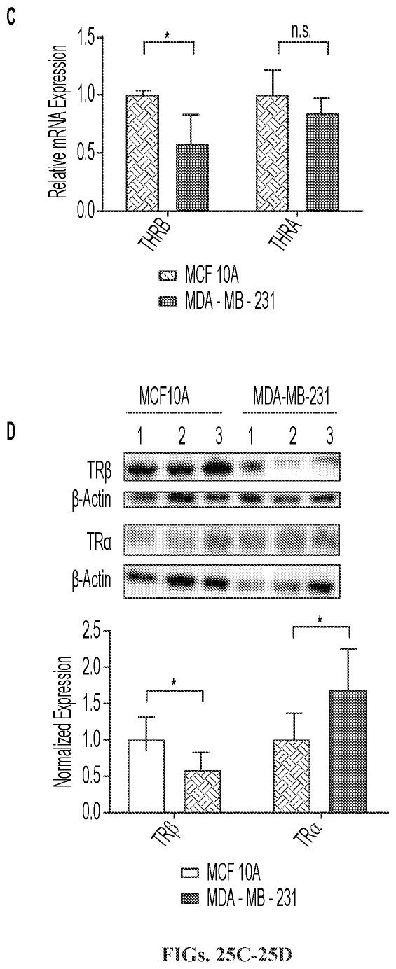

TRβ expression levels are reduced in breast cancers with greatest loss usually associated with most aggressive disease. Differential expression of TRs are reflected in breast cancer cell lines corresponding to different types of breast cancers. As shown in FIG. 25A, THRB expression is significantly reduced in basal-like breast tumors in comparison to all other subtypes. THRA expression is significantly repressed in Basal-like breast cancers and greatest in HER2 expressing tumors as shown in FIG. 25B. Both MCF10A (normal-like) and MDA-MB-231 (triple negative) breast cancer cells express comparable THRA mRNA (n=4,), MCF10A expresses higher levels of THRB mRNA than MDA-MB-231 (n=4, p<0.05) (FIG. 25C). As shown in FIG. 25D, MCF10A breast cancer cells express greater levels of TRβ protein and MDA-MB-231 breast cancer cells express greater levels of TRα protein as measured by immunoblot (n=6, p<0.05). Relative mRNA expression=target/GAPDH.

The inventor has found that ligand activated TRβ in breast cancer cells induces transcriptomic changes (RNAseq) including intracellular signaling pathways both in common with and distinct from thyroid cancer. These findings indicate that selective activation of TRβ using specific agonists like GC-1 should induce tumor suppression signaling through TRβ regulated pathways that are both tumor (tissue) specific and tumor agnostic.

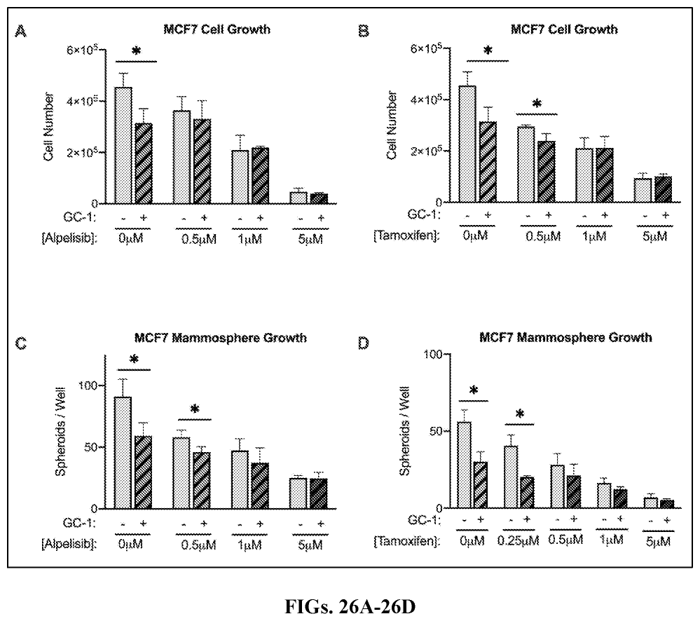

Next, relative cell growth of Luminal A, hormone receptor positive-ER+, breast cancer cells (MCF7) maintained in complete media was measured by cell counting after 8 days of treatment with increasing concentrations Alpelisib (FIG. 26A) or Tamoxifen (FIG. 26B) simultaneously in combination with 10 nM GC-1 (FIG. 26C). It was observed that GC-1 alone could reduce cell growth in MCF7 cells. Further, mammosphere growth was determined for Luminal A breast cancer cells after treatment with 10 nM GC-1 for 24 hours under adherent culture conditions, followed by plating in conditions for spheroid growth in the presence of 10 nM GC-1 and increasing concentrations of Alpelisib (FIG. 26C) or Tamoxifen (FIG. 26D) for 5 days. Data are mean+/−SD; * indicates p<0.05 determined by t-test. It was again observed that GC-1 alone could reduce mammosphere formation in MCF7 cells, and the presence of GC-1 enhanced the effect of Alpelisib, the PI3K inhibitor, and tamoxifen, the selective estrogen receptor modulator.

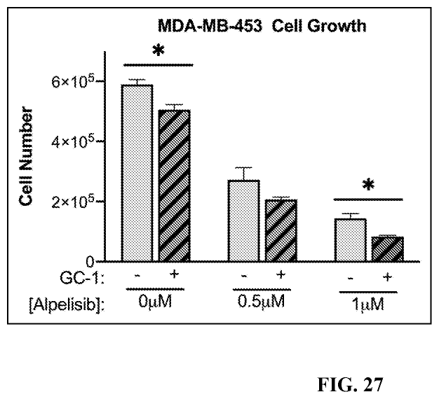

A similar study was then performed in MDA-MB-453 cells, human epidermal growth factor positive-HER2+, breast cancer cells. Relative cell growth of HER2+ breast cells maintained in complete media was measured by cell counting after 8 days of treatment with increasing concentrations Alpelisib simultaneously in combination with 10 nM GC-1. Data are mean+/−SD; * indicates p<0.05 determined by t-test. A shown in FIG. 27, the presence of GC-1 enhanced the effect of Alpelisib in reducing cell growth of HER2+ breast cancer cells. These results indicate that not only does the presence of GC-1 enhance the effectiveness of a therapeutic PI3K inhibitor, Alpelisib, in the more common ER+ breast cancer cells as illustrated by the MCF7, but GC-1 is also effective in increasing the effectiveness of the therapeutic agent in the more aggressive HER2+, ER− breast cancer cells.

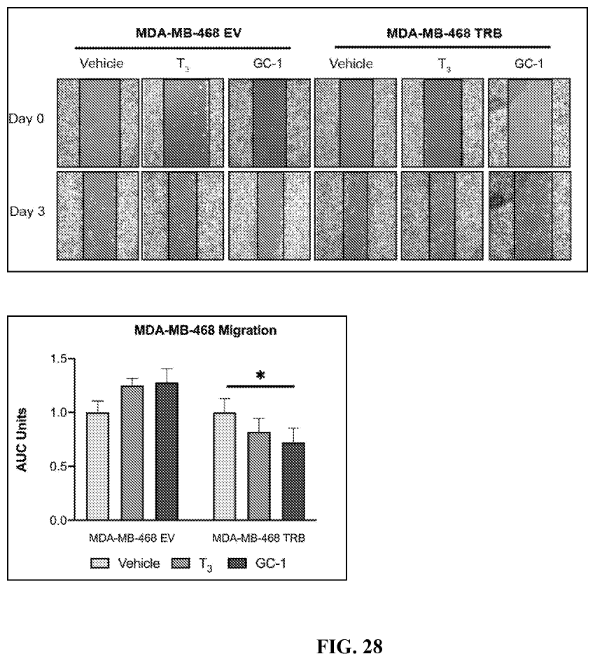

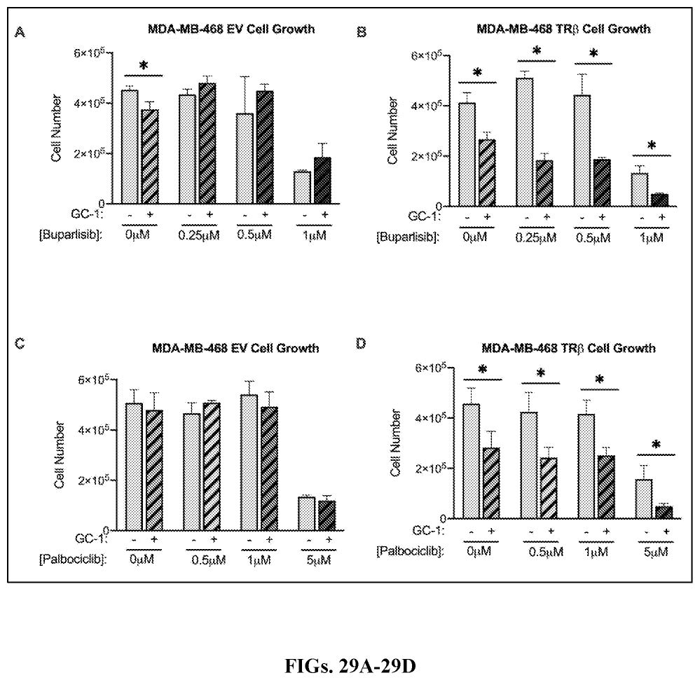

Triple negative breast cancer were examined next. MDA-MB-468 cells were transduced with empty vector (EV) or thyroid hormone receptor beta (TRβ) to assess the impact of GC-1 alone or in combination with therapeutic drugs. TNBC cancers and other advanced solid tumors have lower endogenous levels of expression of TRβ. Re-expression in vitro or longer term treatment with GC-1 is reflected by increased levels of TRβ. Cell migration was measured by scratch assay (FIG. 28). Representative images of scratch closure after 3 days when TNBC cells are treated with 10 nM T₃ or GC-1 are shown in FIG. 28 (top panel). In the lower panel, area under the curve (AUC) analysis of the migration data is summarized normalized to vehicle=1 AUC unit. Data are mean+/−SD; *indicates p<0.05 determined by t-test. These results demonstrate that GC-1 or T₃ reduces the aggressive breast cancer phenotype, reduced cell migration and growth, in the presence of TRβ. In triple negative breast cancer cells, TRβ expression levels are low, nevertheless, GC-1 is able to slow migration (FIG. 28) and cell growth (FIG. 29). With re-expression of TRβ, the effect is magnified (FIG. 29B).

MDA-MB-468 cells were transduced with empty vector (EV) (FIGS. 29A, 29C) or thyroid hormone receptor beta (TRβ) (FIGS. 29B, 29D) and maintained in complete media. Relative cell growth was measured by cell counting after 8 days of treatment with increasing concentrations Buparlisib or Palbociclib simultaneously in combination with 10 nM GC-1(C). Data are mean+/−SD; * indicates p<0.05 determined by t-test. In the presence of GC-1, triple negative breast cancer cells are more sensitive to the pan-PI3K inhibitor Buparlisib (FIG. 29B) or cell cycle inhibitor Palbociclib (FIG. 29D). The combination of GC-1 and Buparlisib or Palbociclib induced a synergistic effect to reduce cell growth respectively, (FIG. 29B, FIG. 29D). In triple negative breast cancer cells, in the presence of TRβ expression, GC-1 increases the efficacy of therapeutic agents.

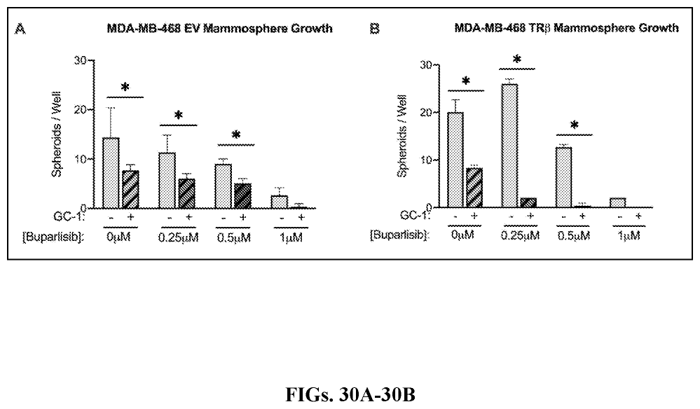

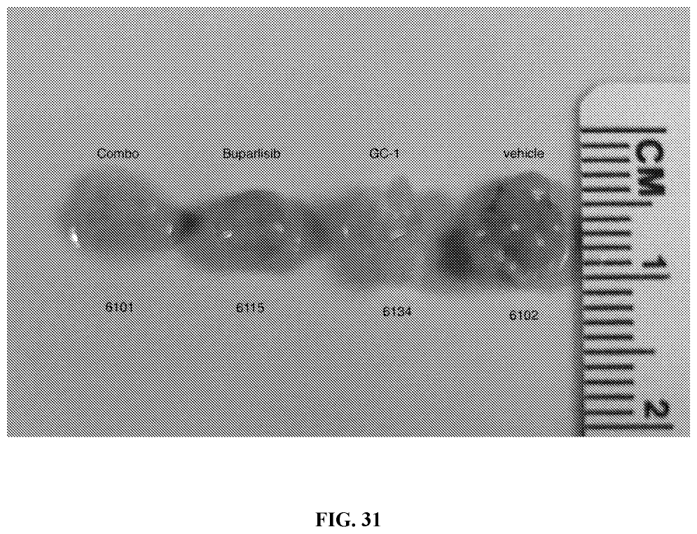

Next, mammosphere growth, a measure of cancer stem cell growth, was determined for the MDA-MB-468 cells transduced with empty vector (EV) (FIG. 30A) or thyroid hormone receptor beta (TRβ) (FIG. 30B) after treatment with 10 nM GC-1 for 24 hours under adherent culture conditions, followed by plating in conditions for spheroid growth in the presence of 10 nM GC-1 and increasing concentrations of Buparlisib for 5 days. Data are mean+/−SD; * indicates p<0.05 determined by t-test. MDA-MB-468-EV mammospheres formed from triple negative breast cancer cells are sensitive to GC-1 (FIG. 30A) reflective of TRβ expression in the cancer stem cell population. Suppression of mammosphere growth by GC-1 is greater in the mammospheres derived from triple negative breast cancer cells with TRβ re-expressed (MDA-MB-468-TRβ) (FIG. 30B). The presence of GC-1 increases the efficacy of Buparlisib to reduced mammosphere growth such that lower concentrations of Buparlisib can be used to reduce cancer stem cell growth. This observation is particularly noted in the mammospheres derived from MDA-MB-468-TRβ cells (FIG. 30B) where effective Buparlisib can be reduced by 50% in the presence of GC-1.

### Example 14—Xenograft Study

The study design was as follows:

Anaplastic Thyroid Cancer
Cells: KTC-2
Vehicle
GC-1 (10⁻⁸M) (0.3 mg/Kg)
Buparlisib (10⁻⁷M)
(5 mg/Kg)
GC-1 + Buparlisib

GC-1 0.3 mg/Kg in DMSO and Buparlisib at 5 mg/Kg were used. GC-1 dose was comparable to “physiological” levels and Buparlisib was comparable to lower doses tested in vitro alone and in combination with GC-1. Doses of Buparlisib tested in xenograft studies range from 5 mg/Kg to 20 mg/Kg.

Drugs were prepared at concentrations suitable for intraperitoneal (i.p.) injection of 5 μL/g body weight. Sobetirome (MW=328 g/mol) and Sob-AM2 (MW=341 g/mol) were synthesized at the Scanlan Laboratory, as previously described. Drug stocks were prepared by dissolving sobetirome and Sob-AM2 at 1 mg/mL in dimethyl sulfoxide (DMSO). The 1 mg/mL drug stocks were diluted in saline to obtain final solutions of 0.2 mg/mL of sobetirome and 0.06 mg/mL of Sob-AM2 in 20% DMSO (corresponding to 1.0 and 0.3 mg/kg dose, respectively). Vehicles were prepared with 20% DMSO in saline.”

Buparlisib was used as a 5 mg/kg buparlisib treatment (recommended by Novartis). Buparlisib suspension was prepared in 0.5% methyl cellulose and 0.5% Tween20.

Tumors were established by subcutaneous injection of 5×10⁶ tumor cells in 100 μL of phosphate-buffered saline into the flank region into SCID female mice. Thyroid cancer occurs in females ˜4× more often than in males. As funding is available; both males and females will be tested.

The study was approximately a 6 week study. Tumor mass was measured, and tumors and tissues were saved for IHC and RNAseq.

As shown in FIG. 31, non-drug treated vehicle mice displayed multinodular primary vascularize tumors as well as metastases to lymph nodes, heart, and liver. GC-1 treated mice displayed smaller growth, minimal vascularization, and no visually detectable metastases. Buparlisib treated mice displayed smaller non-nodular growth but with vascularization and detectable metastases to the heart. The combination of GC-1 and Buparlisib treated mice displayed smaller nodular growth, minimal vascularization, and no visually detectable metastases.

1. A combination therapy comprising:
a thyroid hormone receptor beta-1 (TRβ) agonist, and
a primary cancer therapeutic.

2. The combination therapy of claim 1, wherein the TRβ agonist is selected from the group consisting of 3,5-Dimethyl-4(4′-hydroxy-3′-isopropylbenzyl) phenoxy) acetic acid (sobetirome; GC-1), 2-{4-[(3-benzyl-4-hydroxyphenyl)methyl]-3,5-dimethylphenoxy}acetic acid (GC-24), MGL-3196 (Resmetirom), 2-[4-(4-hydroxy-3-iodophenoxy)-3,5-diiodophenyl]acetic acid (tiratricol), (2S)-2-amino-3-[4-(4-hydroxy-3-iodophenoxy)-3,5-diiodophenyl]propanoic acid (triiodothyronine; T3), (2R)-2-amino-3-[4-(4-hydroxy-3,5-diiodophenoxy)-3,5-diiodophenyl]propanoic acid (dextrothyroxine), 2-[3,5-dichloro-4-(4-hydroxy-3-propan-2-ylphenoxy)phenyl]acetic acid (KB-141), 3-[[3,5-dibromo-4-[4-hydroxy-3-(1-methylethyl)-phenoxy]-phenyl]-amino]-3-oxopropanoic acid (KB2115), (2S)-2-amino-3-[4-(4-hydroxy-3,5-diiodophenoxy)-3,5-diiodophenyl]propanoate (2S)-2-amino-3-[4-(4-hydroxy-3-iodophenoxy)-3,5-diiodophenyl]propanoate (Liotrix), (3,5-dimethyl-4-(4′-hydroxy-3′-isopropylbenzyl)phenoxy)methylphosphonic acid (MB07344), (2R, 4S)-4-(3-chlorophenyl)-2-[(3,5-dimethyl-4-(4′-hydroxy-3′-isopropylbenzyl)phenoxy) methyl]-2-oxido-[1-3]-dioxaphosphonane (Mb07811) and derivatives thereof.

3. The combination therapy of claim 1, wherein the primary cancer therapeutic is an activator of Interferon/JAK1/STAT1 signaling.

4. The combination therapy of claim 3, wherein the activator of Interferon/JAK1/STAT1 signaling is a recombinant interferon-alpha or recombinant interferon-gamma.

5. The combination therapy of claim 3, wherein the activator of Interferon/JAK1/STAT1 signaling is selected from recombinant Oncostatin M and IL-6.

6. The combination therapy of claim 1, wherein the primary cancer therapeutic is an inhibitor of glycogen metabolism.

7. The combination therapy of claim 6, wherein the inhibitor of glycogen metabolism is selected from sodium tungstate, metformin, lithium, valproate, and dichloroacetate.

8. The combination therapy of claim 6, wherein the inhibitor of glycogen metabolism is an inhibitor of glycogen phosphorylase.

9. The combination therapy of claim 8, wherein the inhibitor of glycogen phosphorylase is selected from the group consisting of 5-chloro-N-[(2S,3R)-4-(dimethylamino)-3-hydroxy-4-oxo-1-phenylbutan-2-yl]-1H-indole-2-carboxamide (CP-91149), 5-chloro-N-[(2S,3R)-4-[(3R,4S)-3,4-dihydroxypyrrolidin-1-yl]-3-hydroxy-4-oxo-1-phenylbutan-2-yl]-1H-indole-2-carboxamide (ingliforib), 5-chloro-N-[(2S)-3-(4-fluorophenyl)-1-(4-hydroxypiperidin-1-yl)-1-oxopropan-2-yl]-1H-indole-2-carboxamide (CP-320626), (2R,3S)-2,3-bis[[(E)-3-(4-hydroxyphenyl)prop-2-enoyl]oxy]pentanedioic acid (FR258900), N-(3,5-dimethyl-benzoyl)-N′-((3-D-glucopyranosyl) urea (KB228), 5-chloro-N-[(2S,3R)-3-hydroxy-4-[methoxy(methyl)amino]-4-oxo-1-phenylbutan-2-yl]-1H-indole-2-carboxamide (CP-316819), 5-chloro-N-[3-(4-fluorophenyl)-1-(4-hydroxypiperidin-1-yl)-1-oxopropan-2-yl]-1H-indole-2-carboxamide (CP320626), isopropyl 4-(2-chlorophenyl)-1-ethyl-2-methyl-5-oxo-1,4,5,7-tetrahydro-furo[3,4-b]pyridine-3-carboxylate (BAY R3401), (4S)-1-ethyl-6-methyl-4-phenyl-5-propan-2-yloxycarbonyl-4H-pyridine-2,3-dicarboxylic acid (BAY-W1807), 1,4-dideoxy-1,4-amino-D-arabinitol (DAB), 4-[3-(2-Chloro-4,5-Difluoro-Benzoyl)ureido]-3-Trifluoromethoxybenzoic Acid (AVE5688), GSK1362885, and PSN-357.

10. The combination therapy of claim 1, wherein the primary cancer therapeutic is a phosphoinositide 3-kinase (PI3K) inhibitor.

11. The combination therapy of claim 10, wherein the PI3K inhibitor is selected from the group consisting of 5-(2,6-dimorpholin-4-ylpyrimidin-4-yl)-4-(trifluoromethyl)pyridin-2-amine (buparlisib), 4-morpholino-2-phenylquinazolines, pyrido[3′,2′:4,5]furo[3,2-d]pyrimidine, pyrido[3′,2′:4, 5]furo[3,2-d]pyrimidine, PWT-458 (pegylated-17-hydroxywortmannin), PX-866 (wortmannin analogue), 3-[6-(morpholin-4-yl)-8-oxa-3,5,10-triazatricyclo[7.4.0.0\hat{ }{2,7}]trideca-1(13),2,4,6,9,11-hexaen-4-yl]phenol (PI103), 5-[bis(morpholin-4-yl)-1,3,5-triazin-2-yl]-4-(trifluoromethyl)pyridin-2-amine (PQR-309), (S)-3-(1-((9H-purin-6-yl)amino)ethyl)-8-chloro-2-phenylisoquinolin-1(2H)-one (Duvelisib), N-[4-[[3-(3,5-dimethoxyanilino)quinoxalin-2-yl]sulfamoyl]phenyl]-3-methoxy-4-methylbenzamide (Voxtalisib), 1-[(3S)-3-[[6-[6-methoxy-5-(trifluoromethyl)pyridin-3-yl]-′7,8-dihydro-5H-pyrido[4,3-d]pyrimidin-4-yl]amino]pyrrolidin-1-yl]propan-1-one (Leniolisib), (1,1-dimethylpiperidin-1-ium-4-yl) octadecyl phosphate(perifosine), 5-[7-methanesulfonyl-2-(morpholin-4-yl)-5H,6H,7H-pyrrolo[2,3-d]pyrimidin-4-yl]pyrimidin-2-amine (MEN1611), (2S)-1-N-[4-methyl-5-[2-(1,1,1-trifluoro-2-methylpropan-2-yl)pyridin-4-yl]-1,3-thiazol-2-yl]pyrrolidine-1,2-dicarboxamide (alpelisib), (1S,3S,4R)-4-[(3aS,4R,5S,7aS)-4-(aminomethyl)-7a-methyl-1-methylidene-3,3a,4,5,6,7-hexahydro-2H-inden-5-yl]-3-(hydroxymethyl)-4-methylcyclohexan-1-ol (rosiptor), 2-[6-(1H-indol-4-yl)-1H-indazol-4-yl]-5-[(4-propan-2-ylpiperazin-1-yl)methyl]-1,3-oxazole (nemiralisib), 2-amino-N-[7-methoxy-8-(3-morpholin-4-ylpropoxy)-2,3-dihydroimidazo[1,2-c]quinazolin-5-yl]pyrimidine-5-carboxamide (copanlisib), 2,4-difluoro-N-[2-methoxy-5-[4-methyl-8-[(3-methyloxetan-3-yl)methoxy]quinazolin-6-yl]pyridin-3-yl]benzenesulfonamide (Compound 5d), [6-(2-amino-1,3-benzoxazol-5-yl)imidazo[1,2-a]pyridin-3-yl]-morpholin-4-ylmethanone (serabelisib), 2-(morpholin-4-yl)-8-phenyl-4H-chromen-4-one (LY294002), 3-(3-fluorophenyl)-2-[(1S)-1-[(9H-purin-6-yl)amino]propyl]-4H-chromen-4-one (tenalisib), (2S)-2-[[2-[(4S)-4-(difluoromethyl)-2-oxo-1,3-oxazolidin-3-yl]-5,6-dihydroimidazo[1,2-d][1,4]benzoxazepin-9-yl]amino]propanamide (GDC-0077), 8-(6-methoxypyridin-3-yl)-3-methyl-1-[4-piperazin-1-yl-3-(trifluoromethyl)phenyl]imidazo[5,4-c]quinolin-2-one (BGT 226), [8-[6-amino-5-(trifluoromethyl)pyridin-3-yl]-1-[6-(2-cyanopropan-2-yl)pyridin-3-yl]-3-methylimidazo[4,5-c]quinolin-2-ylidene]cyanamide (panulisib), (5Z)-5-[(4-pyridin-4-ylquinolin-6-yl)methylidene]-1,3-thiazolidine-2,4-dione (GSK1059615), 7-methyl-2-morpholin-4-yl-9-[1-(phenylamino)ethyl]pyrido[2,1-b]pyrimidin-4-one (TGX 221), 4-[6-[[4-(cyclopropylmethyl)piperazin-1-yl]methyl]-2-(5-fluoro-1H-indol-4-yl)thieno[3,2-d]pyrimidin-4-yl]morpholine (PI 3065), 2-(difluoromethyl)-1-[4,6-di(morpholin-4-yl)-1,3,5-triazin-2-yl]benzimidazole (ZSTK474), 1-[4-[4-(dimethylamino)piperidine-1-carbonyl]phenyl]-3-[4-(4,6-dimorpholin-4-yl-1,3,5-triazin-2-yl)phenyl]urea (gedatolisib), 5-fluoro-3-phenyl-2-[(1S)-1-(7H-purin-6-ylamino)propyl]quinazolin-4-one (idelalisib), 3-[2,4-diamino-6-(3-hydroxyphenyl)pteridin-7-yl]phenol (TG-100-115), ethyl 6-[5-(benzenesulfonamido)pyridin-3-yl]imidazo[1,2-a]pyridine-3-carboxylate (HS-173), 2-[[2-methoxy-5-[[(E)-2-(2,4,6-trimethoxyphenyl)ethenyl]sulfonylmethyl]phenyl]amino]acetic acid (rigosertib), 2-(6,7-dimethoxyquinazolin-4-yl)-5-pyridin-2-yl-1,2,4-triazol-3-amine (CP-466722), N-[3-(2,1,3-benzothiadiazol-6-ylamino)quinoxalin-2-yl]-4-methylbenzenesulfonamide (pilaralisib), 2-[(1S)-1-[4-amino-3-(3-fluoro-4-propan-2-yloxyphenyl)pyrazolo[3,4-d]pyrimidin-1-yl]ethyl]-6-fluoro-3-(3-fluorophenyl)chromen-4-one (umbralisib), 5-(8-methyl-2-morpholin-4-yl-9-propan-2-ylpurin-6-yl)pyrimidin-2-amine (VS-5584), 2-methyl-2-[4-(3-methyl-2-oxo-8-quinolin-3-ylimidazo[4,5-c]quinolin-1-yl)phenyl]propanenitrile (dactolisib), 1-(4-{5-[5-amino-6-(5-tert-butyl-1,3,4-oxadiazol-2-yl)pyrazin-2-yl]-1-ethyl-1H-1,2,4-triazol-3-yl}piperidin-1-yl)-3-hydroxypropan-1-one (AZD8835), [(3aR,6E,9S,9aR,10R,11aS)-6-[(di(prop-2-enyl)amino)methylidene]-5-hydroxy-9-(methoxymethyl)-9a,11a-dimethyl-1,4,7-trioxo-2,3,3a,9,10,11-hexahydroindeno[7,6-h]isochromen-10-yl] acetate (sonolisib), 2-[[4-amino-3-(3-fluoro-5-hydroxyphenyl)pyrazolo[3,4-d]pyrimidin-1-yl]methyl]-5-[3-[2-(2-methoxyethoxy)ethoxy]prop-1-ynyl]-3-[[2-(trifluoromethyl)phenyl]methyl]quinazolin-4-one (RV-6153), 6-[2-[[4-amino-3-(3-hydroxyphenyl)pyrazolo[3,4-d]pyrimidin-1-yl]methyl]-3-[(2-chlorophenyl)methyl]-4-oxoquinazolin-5-yl]-N,N-bis(2-methoxyethyl)hex-5-ynamide (RV-1729), 2-(4-ethylpiperazin-1-yl)-N-[4-(2-morpholin-4-yl-4-oxochromen-8-yl)dibenzothiophen-1-yl]acetamide (KU-0060648), N′-cyclopropyl-N-quinolin-6-yl-7H-purine-2,6-diamine (puquitinib).

12. The combination therapy of claim 1, wherein the primary cancer therapeutic is a PTEN activator.

13. The combination therapy of claim 12, wherein the PTEN activator is an antibody selected from an anti-CD20 antibody (Ublituximab, Rituximab), a HER2 antibody (Trastuzumab, Pertuzumab), and an epidermal growth factor receptor antibody (Cetuximab).

14. The combination therapy of claim 12, wherein the PTEN activator is small molecule activator selected from N-[2-(diethylamino)ethyl]-5-{[(3Z)-5-fluoro-2-oxo-2,3-dihydro-1H-indol-3-ylidene]methyl}-2,4-dimethyl-1H-pyrrole-3-carboxamide N-[2-(diethylamino)ethyl]-5-{[(3Z)-5-fluoro-2-oxo-2,3-dihydro-1H-indol-3-ylidene]methyl}-2,4-dimethyl-1H-pyrrole-3-carboxamide (Sunitinib), N-(3-chloro-4-fluorophenyl)-7-methoxy-6-[3-(morpholin-4-yl)propoxy]quinazolin-4-amine (Gefitnib), N-(3-ethynylphenyl)-6,7-bis(2-methoxyethoxy)quinazolin-4-amine (Erlotinib), [(1S,3R,7S,8S,8aR)-8-[2-[(2R,4R)-4-hydroxy-6-oxooxan-2-yl]ethyl]-3,7-dimethyl-1,2,3,7,8,8a-hexahydronaphthalen-1-yl] 2,2-dimethylbutanoate (Simvastatin), [(1S,3R,7S,8S,8aR)-8-[2-[(2R,4R)-4-hydroxy-6-oxooxan-2-yl]ethyl]-3,7-dimethyl-1,2,3,7,8,8a-hexahydronaphthalen-1-yl] (2S)-2-methylbutanoate (Lovastatin), 5-[[4-[2-(methyl-pyridin-2-ylamino)ethoxy]phenyl]methyl]-1,3-thiazolidine-2,4-dione (Rosiglitazone), 7-[3-(azetidin-1-ylmethyl)cyclobutyl]-5-[3-(phenylmethoxy)phenyl]pyrrolo[3,2-e]pyrimidin-4-amine (NVP-AEW541), (9 S,12S,14S,16S,17R)-8,13,14,17-tetramethoxy-4,10,12,16-tetramethyl-3,20,22-trioxo-2-azabicyclo[16.3.1]docosa-1(21),4,6,10,18-pentaen-9-yl carbamate (Herbimycin)

15. The combination therapy of claim 1, wherein the primary cancer therapeutic is an anti-estrogen.

16. The combination therapy of claim 15, wherein the anti-estrogen is selected from fulvestrant, tamoxifen, clomifene, raloxifene and toremifene

17. The combination therapy of claim 15, wherein the primary therapeutic is a MAPK inhibitor selected from the group consisting of a KRAS inhibitor, a BRAF inhibitor, a MEK inhibitor, and an ERK inhibitor.

18. The combination therapy of claim 17, wherein the primary therapeutic is a KRAS inhibitor selected from AMG-510 and MRTX849.

19. The combination therapy of claim 17, wherein the primary therapeutic is a BRAF inhibitor selected from sorafenib, vemurafenib, dabrafenib

20. The combination therapy of claim 17, wherein the primary therapeutic is a MEK inhibitor selected from selumentinib and tramentinib.

21. The combination therapy of claim 17, wherein the ERK inhibitor is selected from ulixertinib and silymarin (rapamycin).

22. The combination therapy of claim 1, wherein the primary cancer therapeutic is an inhibitor of cancer stem cell formation.

23. The combination therapy of claim 17, wherein the primary cancer therapeutic is bevacizumab, Ranibizumab, Aflibercept, Sunitinib, Pazopanib, Axitinib, Regorafenib, Sorafenib, Lenvatinib, and Nintedanib.

24. The combination therapy of claim 1, wherein the primary cancer therapeutic is a cyclin dependent kinase (CDK) inhibitor.

25. The combination therapy of claim 19, wherein the CDK inhibitor is a CDK4/6 inhibitor.

26. The combination therapy of claim 20, wherein the CDK inhibitor is selected from 6-acetyl-8-cyclopentyl-5-methyl-2-[(5-piperazin-1-ylpyridin-2-yl)amino]pyrido[6,5-d]pyrimidin-7-one (palbociclib), 7-cyclopentyl-N,N-dimethyl-2-{[5-(piperazin-1-yl)pyridin-2-yl]amino}-7H-pyrrolo[2,3-d]pyrimidine-6-carboxamide (ribociclib), N-[5-[(4-ethylpiperazin-1-yl)methyl]pyridin-2-yl]-5-fluoro-4-(7-fluoro-2-methyl-3-propan-2-ylbenzimidazol-5-yl)pyrimidin-2-amine (Abemaciclib), 2-[[5-(4-methylpiperazin-1-yl)pyridin-2-yl]amino]spiro[7,8-dihydropyrazino[5,6]pyrrolo[1,2-d]pyrimidine-9,1′-cyclohexane]-6-one (Trilaciclib), Alvocidib, and Fostamatinib

27. The combination therapy of claim 1, wherein the TRβ agonist and the primary cancer therapeutic are formulated together in a single pharmaceutical composition.

28. The combination therapy of claim 1, wherein the TRβ agonist and the primary cancer therapeutic are formulated as separate pharmaceutical compositions.

29. A method of treating cancer in a subject, said method comprising:
administering to a subject having a cancer, wherein the cancer is characterized by cells having a decreased level of thyroid hormone receptor beta-1 (TRβ) expression or activity relative to corresponding non-cancer cells of similar origin, a TRβ agonist in an amount effective to treat the cancer.

30.-66. (canceled)

67. A method of inducing differentiation in a population of cancer cells, said method comprising:
administering to a population of cancer cells having a decreased level of thyroid hormone receptor beta-1 (TRβ) expression or activity relative to a corresponding population of non-cancer cells of similar origin, a TRβ agonist in an amount effective to induce differentiation of said cancer cells of the population.

68.-103. (canceled)


SEQUENCE LISTING (16 sequences)

SEQ ID NO: 1 - protein, 195 aa, Artificial
  recombinant oncostatin M protein
AAIGSCSKEYRVLLGQLQKQTDLMQDTSRLLDPYIRIQGLDVPKLREHCRERPGAFPSEETLRGLGRRGFLQTLNATLGCVLHRLADLEQRLPKAQDLERSGLNIEDLEKLQMARPNILGLRNNIYCMAQLLDNSDTAEPTKAGRGASQPPTPTPASDAFQRKLEGCRFLHGYHRFMHSVGRVFSKWGESPNRSR

SEQ ID NO: 2 - protein, 183 aa, Artificial
  recombinant IL-6 protein
VPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 3 - DNA, 22 bp, Artificial
  MYC forward oligo
agcgactctgaggagggaacaa

SEQ ID NO: 4 - DNA, 20 bp, Artificial
  MYC reverse oligo
gtgggctgtgaggaggtttg

SEQ ID NO: 5 - DNA, 21 bp, Artificial
  CDKN1A forward oligo
gcagaccagcatgacagattt

SEQ ID NO: 6 - DNA, 21 bp, Artificial
  CDKN1A reverse oligo
ctggacttcgagcaagagatg

SEQ ID NO: 7 - DNA, 24 bp, Artificial
  TNFSF10 forward oligo
ccgtcagctcgttagaaagatgat

SEQ ID NO: 8 - DNA, 21 bp, Artificial
  TNFSF10 reverse oligo
tgtgttgcttcttcctctggt

SEQ ID NO: 9 - DNA, 20 bp, Artificial
  APOL6 forward oligo
cctgttcaaaggccacccta

SEQ ID NO: 10 - DNA, 25 bp, Artificial
  APOL6 reverse oligo
tcaaatgattttcttctctccacgg

SEQ ID NO: 11 - DNA, 20 bp, Artificial
  TAP1 forward oligo
gccttgttccgagagctgat

SEQ ID NO: 12 - DNA, 20 bp, Artificial
  TAP1 reverse oligo
aatggccatctccccaagag

SEQ ID NO: 13 - DNA, 20 bp, Artificial
  IRF1 forward oligo
cttccaggtgtcacccatgc

SEQ ID NO: 14 - DNA, 20 bp, Artificial
  IRF1 reverse oligo
ccatccacgtttgttggctg

SEQ ID NO: 15 - DNA, 20 bp, Artificial
  GAPDH forward oligo
atgttcgtcatgggtgtgaa

SEQ ID NO: 16 - DNA, 20 bp, Artificial
  GAPDH reverse oligo
tgtggtcatgagtccttcca